(19) United States
(12) Patent Application Publication
(10) Pub. No.: US 2021/0402002 A1
(43) Pub. Date: Dec. 30, 2021

(54) CONJUGATE

(71) Applicant: GLYKOS BIOMEDICAL OY, Helsinki (FI)

(72) Inventors: Juhani SAARINEN, Helsinki (FI); Tero SATOMAA, Helsinki (FI); Olli AITIO, Helsinki (FI)

(73) Assignee: GLYKOS BIOMEDICAL OY, Helsinki (FI)

(21) Appl. No.: 17/251,657

(22) PCT Filed: Jun. 19, 2019
(86) PCT No.: PCT/FI2019/050479

§ 371 Date: Dec. 11, 2020

(30) Foreign Application Priority Data
Jun. 19, 2018 (FI) 20185553
Feb. 28, 2019 (FI) 20195151

(51) Int. Cl. A61K 47/68; A61P 35/00

(52) U.S. Cl. CPC A61K 47/6855; A61P 35/00

(57) ABSTRACT

A conjugate is disclosed. The conjugate may comprise a targeting unit for delivery to a tumour, and a glycosylation inhibitor for inhibiting glycosylation in the tumour, thereby decreasing the immunosuppressive activity of the tumour. The glycosylation inhibitor may be conjugated to the targeting unit.

### TECHNICAL FIELD

The present disclosure relates to a conjugate.

### BACKGROUND

Immunotherapy for cancer may employ the body's own immune system to recognize and eradicate cancer cells. However, tumour cells, such as cancer cells, may utilize several mechanisms to suppress the activity of cells of the immune system of the subject having the tumour. Means for decreasing the immunosuppressive activity of malignant or cancer cells and/or for boosting immune responses of the subject may therefore improve cancer immunotherapy (Pardoll, Nat. Rev. Cancer 12:252-64, 2012). Combination of targeted therapy to immunotherapy may further improve treatment outcomes (Vanneman & Dranoff, Nat. Rev. Cancer 12:237-51, 2012).

### SUMMARY

A conjugate is disclosed. The conjugate may comprise a targeting unit for delivery to a tumour, and a glycosylation inhibitor for inhibiting glycosylation in the tumour, thereby decreasing the immunosuppressive activity of the tumour. The glycosylation inhibitor may be conjugated to the targeting unit.

### BRIEF DESCRIPTION OF THE DRAWINGS

The accompanying drawings, which are included to provide a further understanding of the embodiments and constitute a part of this specification, illustrate various embodiments. In the drawings:

FIG. 1 illustrates the MALDI-TOF mass spectrum of 6-succinyl-4-F-GlcNAc reaction products, showing expected mass for 6-succinyl-4-F-GlcNAc at m/z 346 [M+Na]⁺.

FIG. 2 shows the MALDI-TOF mass spectrum of purified 6-succinyl-4-F-GlcNAc, with the product ion at m/z 346 [M+Na]⁺.

FIG. 3 shows the MALDI-TOF mass spectrum of DBCO-6-succinyl-4-F-GlcNAc, with the product ion at m/z 604 [M+Na]⁺.

FIG. 4 shows the successful generation of azide-modified trastuzumab, 2 azides/antibody, wherein N-azidoacetylgalactosamine (GalNAz) residues were transferred to N-glycan core N-acetylglucosamine residues with mutant galactosyltransferase reaction after cleaving the N-glycans by endoglycosidase S2. The MALDI-TOF mass spectrum of the heavy chain Fc domain was recorded after isolation of the fragments by Fabricator enzyme digestion showed the expected m/z values after (A) endoglycosidase digestion and (B) galactosyltransferase reaction. Closed square, GlcNAc; open square with azide, GalNAz; closed triangle, fucose; gray ovals, heavy chain Fc domain fragment.

FIG. 5 shows effective inhibition of SKOV3 cancer cell surface sialylation by peracetylated 3-fluoro-sialic acid (P-3Fax-Neu5Ac), as detected with fluorescein-labeled lectin SNA-I-FITC by fluorescence-assisted cell sorting (FACS). Lectin staining drops after incubation with the sialylation inhibitor compared to untreated cells. Untreated cells=light grey histogram; Inhibitor-treated cells=dark grey histogram; Control=black line.

FIG. 6 shows effective inhibition of SKOV3 cancer cell surface Galectin ligand expression by peracetylated 4-fluoro-N-acetylglucosamine (P-4F-GlcNAc), as detected with fluorescein-labeled lectin LEA-FITC as well as Alexa Fluor 488-conjugated Galectin-1 and Galectin-3 by FACS. Lectin and Galectin staining drops after incubation with the glycosylation inhibitor compared to untreated cells. Untreated cells=light grey histogram; Inhibitor-treated cells=dark grey histogram; Control=black line.

FIG. 7 shows effective inhibition of sialylated Siglec ligand glycan biosynthesis and expression on the surface of HSC-2 cancer cells by P-3Fax-Neu5Ac, as detected with fluorescein-labeled lectin SNA-I and Siglec-7 by FACS. The staining drops after incubation with the glycosylation inhibitor compared to untreated cells. Untreated cells=light grey histogram; Inhibitor-treated cells=dark grey histogram; Control=black line.

FIG. 8 shows effective inhibition of Galectin ligand glycan biosynthesis in and expression on the surface of HSC-2 cancer cells by P-4F-GlcNAc, as detected with fluorescein-labeled lectin and Galectin-1 in FACS. The staining drops after incubation with the glycosylation inhibitor compared to untreated cells. Untreated cells=light grey histogram; Inhibitor-treated cells=dark grey histogram; Control=black line.

FIG. 9 shows successful generation of glycosylation inhibitor-antibody conjugates (ADCs), formed by conjugation of maleimide-linker-drugs to reduced hinge region cysteines, as analyzed by MALDI-TOF MS. A. Trastuzumab-MC-VC-PAB-4-F-GlcN, DAR=4-8. B. C. Trastuzumab control. D. Trastuzumab-MC-VC-PAB-4-F-GlcNAc glycosylamine, DAR=4-8. E. Trastuzumab-MC-VC-PAB-3Fax-Neu5N, DAR=4-8. F. Trastuzumab-MC-VC-PAB-1-deoxymannojirimycin, DAR=8. G. Trastuzumab-MC-VC-PAB-DMAE-kifunensine, DAR=4-8. The mass spectra of the antibody fragments were recorded after Fabricator enzyme digestion.

FIG. 10 shows effective inhibition of sialylated Siglec ligand glycan and N-glycan biosynthesis in cancer cells by glycosylation inhibitor-ADCs, as detected with fluorescein-labeled lectin SNA-I by FACS. A. SKBR-3 breast cancer cells were incubated for four days with 500 nM trastuzumab-MC-VC-PAB-3Fax-Neu5N, DAR=4-8, and analyzed with SNA-I in FACS. The staining dropped after incubation with the ADC compared to untreated cells, showing inhibition of cell surface sialylation. B. SKBR-3 cells were incubated for four days with 10 nM Trastuzumab-MC-VC-PAB-DMAE-kifunensine, DAR=4-8, and analyzed with SNA-I in FACS. The staining dropped after incubation with the ADC compared to untreated cells, showing inhibition of N-glycosylation-associated cell surface sialylation. Untreated cells=light grey histogram; Inhibitor-treated cells=dark grey histogram; Control=black line.

FIG. 11 shows inhibition of HER2 glycoprotein N-glycosylation in SKBR-3 cells by trastuzumab-MC-vc-PAB-DMAE-tunicamycin DAR=8 ADC (A. and C.) and tunicamycin (B. and D.) after six days' incubation. Increasing concentration of A. tunicamycin-ADC and B. tunicamycin decreased relative MW of HER2 in SDS-PAGE corresponding to defective N-glycosylation. EC50 (concentration with 50% efficacy) of the effect was C. 40 nM for tunicamycin-ADC and D. 70 nM for tunicamycin.

FIG. 12 shows viability assay results of trastuzumab-MC-vc-PAB-DMAE-tunicamycin DAR=8 ADC (Tmab-Tuni DAR=8 ADC, solid line and closed circles), trastuzumab (Tmab, dashed line and open triangles) and omalizumab-MC-vc-PAB-DMAE-tunicamycin DAR=8 ADC (Omab-Tuni DAR=8 ADC, open circles), with A. SKBR-3 cells cultured for five days and B. for eight days with the molecules. Tmab-Tuni DAR=8 ADC had IC50 (concentration with 50% inhibition of cellular viability) of 130 nM at five days and 90 nM at eight days, while both trastuzumab and Omab-Tuni DAR=8 ADC did not reach IC50 at 1 μM concentration.

### DETAILED DESCRIPTION

### Outline of Sections

I) Definitions

II) Glycosylation inhibitors

III) Linker units

IV) Targeting units

V) Stretcher units

VI) Specificity units

VII) Spacer units

VIII) Further linker units

IX) Conjugates

X) Compositions and methods

I) Definitions

A conjugate is disclosed.

The conjugate may comprise a targeting unit for delivery to a tumour, and a glycosylation inhibitor for inhibiting glycosylation in the tumour, thereby decreasing the immunosuppressive activity of the tumour.

The conjugate may be a conjugate for decreasing the immunosuppressive activity of a target cell, which is a tumour cell, and/or of a second tumour cell.

The conjugate may thus comprise a targeting unit for delivery to the tumour, and a glycosylation inhibitor for inhibiting glycosylation in the tumour, for example in the target cell or in the second tumour cell, thereby decreasing the immunosuppressive activity of the tumour, for example the immunosuppressive activity of the target cell and/or of the second tumour cell.

The glycosylation inhibitor may be conjugated to the targeting unit. The glycosylation inhibitor may be conjugated to the targeting unit at least partially covalently. For example, it may be conjugated covalently, or partially non-covalently (and partially covalently).

Many tumours are known to be formed of not only malignant or cancer cells, but also of non-malignant or non-cancer cells of the subject having the tumour. Such non-malignant or non-cancer cells may be migrated to the tumour, so that they are located within the tumour or the tumour microenvironment or otherwise be intimately associated with the tumour. For example, such non-malignant or non-cancer cells may be located between the malignant or cancer cells, or they may be in direct physical contact with the malignant or cancer cells.

In the context of this specification, the term “tumour cell” may refer to any cell of any cell type that forms a part of or is associated with a tumour. The term may encompass malignant or cancer cells and, additionally or alternatively, non-cancer or non-malignant cells that form a part of or are associated with the tumour. The term may also encompass any non-cancer or non-malignant cell present in the tumour microenvironment. The tumour cells may include, for example, cells of the immune system. Examples of such tumour cells may include tumour infiltrating immune cells, such as tumour infiltrating lymphocytes, cells of the immune system, cells of the tumour vasculature and lymphatics, as well as fibroblasts, pericytes and adipocytes. Specific examples of such non-cancer tumour cells may include T cells (T lymphocytes); CD8+ cells including cytotoxic CD8+ T cells; CD4+ cells including T helper (TH1) cells, TH2 cells, TH17 cells, Tregs; γδ T lymphocytes; B lymphocytes including B cells and Bregs (B10 cells); NK cells; NKT cells; tumour-associated macrophages (TAMs); myeloid-derived suppressor cells (MDSCs); dendritic cells (DCs); tumour-associated neutrophils (TANs); CD11b+ bone-marrow-derived myeloid cells; fibroblasts including myofibroblasts and cancer-associated fibroblasts; endothelial cells; smooth muscle cells; myoepithelial cells; stem cells including multipotent stem cells, lineage-specific stem cells, progenitor cells, pluripotent stem cells, cancer stem cells (cancer-initiating cells), mesenchymal stem cells and hematopoietic stem cells; adipocytes; vascular endothelial cells; stromal cells; perivascular stromal cells (pericytes); and lymphatic cells including lymphatic endothelial cells (Balkwill et al. 2012. J. Cell Sci. 125:5591-6), provided they form a part of or are associated with the tumour.

In other words, the tumour cells, which thus may form a tumour, may comprise at least malignant or cancer cells and non-cancer or non-malignant cells that form a part of or are associated with the tumour. The target cell may be at least one of the malignant or cancer cells or the non-cancer or non-malignant cells (for example, cells of the immune system). Likewise, the second tumour cell may be at least one of the malignant or cancer cells or the non-cancer or non-malignant cells (for example, cells of the immune system).

The targeting unit may be suitable for delivery to the tumour in various ways, for example for binding the tumour, e.g. the target cell or a molecule within the tumour.

In an embodiment, the targeting unit may bind or be capable of binding to a tumour molecule, thereby facilitating the delivery of the conjugate to the tumour or to any cells of the tumour.

In the context of this specification, the term “tumour molecule” may refer to any molecule of any molecule type that forms a part of or is associated (for example, intimately associated) with a tumour. The term may encompass molecules produced by the malignant or cancer cells and, additionally or alternatively, molecules produced by the non-cancer or non-malignant cells that form a part of or are associated with the tumour and, additionally or alternatively, molecules that are produced by non-tumour cells and that form a part of or are associated with the tumour. The term may also encompass any molecule present in the tumour microenvironment. The tumour molecules may include, for example, proteins, lipids, glycans, nucleic acids, or combinations thereof. The tumour molecule may, in some embodiments, be specific to the tumour or enriched in the tumour.

Upon or after binding to a tumour molecule, the conjugate may release the glycosylation inhibitor, such that the glycosylation inhibitor may, for example, enter or otherwise interact with the target cell or, in some embodiments, the second tumour cell.

By inhibiting glycosylation in the tumour, for example in the target cell, the conjugate may be capable of decreasing the immunosuppressive activity of the tumour, for example of the target cell. However, additionally or alternatively, by inhibiting glycosylation in the target cell, the conjugate may be capable of decreasing the immunosuppressive activity of the second tumour cell. For example, the inhibition may cause the target cell to have altered glycosylation structures, e.g. as a part of membrane-bound or secreted tumour proteins. These altered glycosylation structures may then interact with the second tumour cell within the tumour microenvironment, thereby decreasing the immunosuppressive activity of the second tumour cell.

In an embodiment, the conjugate is a conjugate for decreasing the immunosuppressive activity of the target cell.

In an embodiment, the conjugate is a conjugate for decreasing the immunosuppressive activity of the second tumour cell.

In an embodiment, the conjugate is a conjugate for decreasing the immunosuppressive activity of the target cell and of the second tumour cell.

The tumour cells may have immunosuppressing receptors. The conjugate may thus be suitable for decreasing, or configured to decrease, the immunosuppressive activity of the tumour, e.g. of the target cell and/or of the second tumour cell, for example by reducing the activity of one or more of the immunosuppressing receptors of the the target cell and/or of the second tumour cell. In an embodiment, the conjugate may be suitable for reducing, or configured to reduce, glycosylation-cellular receptor interactions, for example glycosylation-lectin interactions. The conjugate may thereby reduce immunosuppression by reducing the activity of one or more of the immunosuppressing receptors of the the target cell and/or of the second tumour cell.

In an embodiment, the conjugate is suitable for decreasing, or configured to decrease, interactions between immunosuppressive receptors and glycan ligands of the target cell and/or of the second tumour cell.

In an embodiment, the conjugate is suitable for decreasing, or configured to decrease, Galectin-Galectin ligand interactions and/or Siglec-Siglec ligand interactions. The term “Siglec” may be understood as referring to any sialic acid-recognizing receptor within the Siglec subgroup of mammalian I-type lectins. There are at least 17 Siglecs discovered in mammals, of which at least Siglec-1, -2, -3, -4, -5, -6, -7, -8, -9, -10, -11, -12, -14, -15, -16 and -17 have been identified in humans (Varki et al., eds., Essentials of Glycobiology, 2017, 3rd edition, Cold Spring Harbor Laboratory Press, New York; Chapter 35). The term “Galectin” may be understood as referring to any S-type lectin, which is a galactoside-recognizing receptor. There are at least 15 Galectins discovered in mammals, encoded by the LGALS genes, of which at least Galectin-1, -2, -3, -4, -7, -8, -9, -10, -12 and -13 have been identified in humans (Essentials of Glycobiology 2017; Chapter 36).

The conjugate may thus be suitable for increasing, or configured to increase, the activity of the target cell, which may be a cell of the immune system, against the second tumour cell, such as a malignant or cancer cell.

The conjugate may thus be suitable for increasing, or configured to increase, the activity of the second tumour cell, which may be a cell of the immune system, against the target cell, such as a malignant or cancer cell.

As the glycosylation inhibitor and the targeting unit are conjugated at least partially covalently, it may assist in delivering the glycosylation inhibitor to the target cell and/or to the second tumour cell. The conjugate may also exhibit improved pharmacodynamics and/or pharmacokinetics. Preparing of the conjugate may also be relatively feasible and cost-effective.

In the context of this specification, the term “tumour” may refer to a solid tumour, a diffuse tumour, a metastasis, a tumour microenvironment, a group of tumour cells, a single tumour cell and/or a circulating tumour cell.

In the context of this specification, the term “target cell” may refer to one or more embodiments of the tumour cells, including malignant or cancer cells and/or non-malignant or non-cancer cells, for example cells of the immune system. The target cell may refer to one or more of the tumour cell types. In an embodiment, the target cell may be at least one of a malignant or cancer cell or a non-malignant or non-cancer cell. In an embodiment, the target cell may be a malignant or cancer cell. In an embodiment, the target cell may be a tumour cell that is non-malignant or non-cancer cell, such as a tumour-infiltrating immune cell. The conjugate or a part thereof, for example the glycosylation inhibitor, may subsequently be transported or otherwise move to other tumour cells. Additionally or alternatively, the target cell may be a non-malignant or non-cancer cell, such as a tumour-infiltrating immune cell, and the glycosylation inhibitor may inhibit glycosylation in the target cell itself, thereby reducing the activity of at least a part of the immunosuppressing receptors of the target cell.

In the context of this specification, the term “second tumour cell” may refer to one or more embodiments of the tumour cells, including malignant or cancer cells and/or non-malignant or non-cancer cells, for example cells of the immune system. The second tumour cell may refer to or comprise one or more of the tumour cell types. In an embodiment, the second tumour cell may be at least one of a malignant or cancer cell or a non-malignant or non-cancer cell. In an embodiment, the second tumour cell may be a malignant or cancer cell. In an embodiment, the second tumour cell may be a tumour cell that is non-malignant or non-cancer cell, such as a tumour-infiltrating immune cell.

In the context of this specification, the term “target molecule” may refer to one or more embodiments of the tumour molecules.

In the context of this specification, the term “targeting unit” may refer to a group, moiety or molecule capable of recognizing and binding to the target cell or the target molecule.

The targeting unit may be capable of binding to the target cell specifically. The targeting unit may be capable of binding to the target molecule specifically.

In the context of this specification, the term “glycosylation inhibitor” may refer to any group, moiety or molecule which is capable of inhibiting glycosylation in the target cell or in the second tumour cell, to which the conjugate or a part thereof may be transported or otherwise moved after binding to the target cell or the target molecule. As glycosylation is a complex process involving various biosynthetic steps and mechanisms, the glycosylation inhibitor may in principle inhibit any step or aspect of the glycosylation, such that it decreases, interferes with or prevents the incorporation of glycan structures at the cell surface of one or more embodiments of the tumour cells, for example into glycoproteins and/or glycolipids.

In the context of this specification, the term “to conjugate” or “conjugated” may be understood as referring to linking groups, moieties or molecules, for example the glycosylation inhibitor and the targeting unit, to each other least partially covalently; however such that the linking may, in some embodiments, be arranged at least partially non-covalently. For example, the targeting unit and the glycosylation inhibitor may be conjugated via a linker unit, such that separate ends of the linker unit are conjugated covalently to the targeting unit and to the glycosylation inhibitor, respectively. The targeting unit and the glycosylation inhibitor may, in an embodiment, be conjugated covalently.

However, they may be conjugated such that at least a part of the linker unit may comprise units, groups, moieties or molecules that are linked non-covalently, for example via a non-covalent interaction. An example of such a non-covalent interaction may be biotin-avidin interaction or other non-covalent interaction with a sufficient affinity.

A sufficient affinity for the non-covalent linkage or non-covalent interaction may be e.g. one having a dissociation constant (Kd) in the order of nanomolar Kd, picomolar Kd, femtomolar Kd, attomolar Kd, or smaller. In an embodiment, the affinity is substantially the same as the affinity of biotin-avidin interaction. The affinity may be an affinity with a Kd of about 10⁻¹⁴ mol/l, or to a Kd between 10⁻¹⁵ mol/1 and 10⁻¹² mol/1 (femtomolar), or a Kd below 10⁻¹⁵ mol/1 (attomolar). In an embodiment, the affinity is substantially the same as the affinity of an antibody-antigen interaction, such as an affinity having a Kd of about 10⁻⁹ mol/1, or a Kd of between 10⁻¹² mol/1 and 10⁻⁹ mol/1 (picomolar), or a Kd of between 10⁻⁹ mol/1 and 10⁻⁷ mol/1 (nanomolar). In an embodiment, the affinity may be an affinity with a Kd that is below 10⁻⁷ mol/1, below 10⁻⁸ mol/1, below 10⁻⁹ mol/l, below 10⁻¹⁰ mol/l, below 10⁻¹¹ mol/l, below 10⁻¹² mol/l, below 10⁻¹³ mol/l, below 10⁻¹⁴ mol/l, or below 10⁻¹⁵ mol/l.

In the context of this specification, the terms “SK-BR-3 cell” and “SKBR-3 cell” can be used interchangeably and can be understood referring to the same cell line.

The conjugate may comprise one or more chemical substituents as described by the variables of the chemical formulas of the present disclosure. A person skilled in the art is able to determine what structures are encompassed in the specific substituents based on their names. In the context of this specification, the term “to substitute” or “substituted” may be understood as referring to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known to a person skilled in the art.

In the context of the present specification, the substituents may further comprise certain chemical structures as described in the following embodiments.

In an embodiment, the term “alkyl” means a monovalent moiety obtained or obtainable by removing a hydrogen atom from a carbon atom of a hydrocarbon compound, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, and the like. In an embodiment, the term “C₁₋₁₂ alkyl” means an alkyl moiety having from 1 to 12 carbon atoms.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆) and n-heptyl (C₇).

Examples of saturated branched alkyl groups include isopropyl (C₃), iso-butyl (C₄), sec-butyl (C₄), tert-butyl (C₄), isopentyl (C₅), and neo-pentyl (C₅).

In an embodiment, the term “alkenyl” means an alkyl group having one or more carbon-carbon double bonds. In an embodiment, the term “C₂₋₁₂ alkenyl” means an alkenyl moiety having from 2 to 12 carbon atoms.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

In an embodiment, the term “alkynyl” means an alkyl group having one or more carbon-carbon triple bonds. In an embodiment, the term “C₂₋₁₂ alkynyl” means an alkynyl moiety having from 2 to 12 carbon atoms.

Examples of unsaturated alkynyl groups include, but are not limited to, ethynyl (ethinyl, —C═CH) and 2-propynyl (propargyl, —CH₂—C═CH).

In an embodiment, the term “cycloalkyl” means an alkyl group which is also a cyclyl group; that is, a monovalent moiety obtained by removing a hydrogen atom from an alicyclic ring atom of a cyclic hydrocarbon (carbocyclic) compound. In an embodiment, the term “C₃₋₂° cycloalkyl” means a cycloalkyl moiety having from 3 to 20 carbon atoms, including from 3 to 8 ring atoms.

Examples of cycloalkyl groups include, but are not limited to, those derived from:

saturated monocyclic hydrocarbon compounds: cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);

unsaturated monocyclic hydrocarbon compounds: cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and

saturated polycyclic hydrocarbon compounds: norcarane (C₇), norpinane (C₇), norbornane (C₇).

In an embodiment, the term “heterocyclyl” means a monovalent moiety obtained by removing a hydrogen atom from a ring atom of a heterocyclic compound, which moiety has from 3 to 20 ring atoms, of which from 1 to 10 are ring heteroatoms. In an embodiment, each ring has from 3 to 8 ring atoms, of which from 1 to 4 are ring heteroatoms.

In this context, the prefixes (e.g. C₃₋₂₀, C₃₋₈, C₅₋₆, etc.) denote the number of ring atoms, or range of number of ring atoms, whether carbon atoms or heteroatoms. For example, the term “C₅₋₆ heterocyclyl”, means a heterocyclyl group having 5 or 6 ring atoms.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (trioxane) (C₆); and,

N₁O₁S₁: oxathiazine (C₆).

Examples of substituted monocyclic heterocyclyl groups include those derived from saccharides, in cyclic form, for example, furanoses (C₅), such as arabinofuranose, ribofuranose, and xylofuranose, and pyranoses (C₆), such as fucopyranose, glucopyranose, mannopyranose, idopyranose, and galactopyranose.

In an embodiment, the term “aryl” means a monovalent moiety obtained by removing a hydrogen atom from an aromatic ring atom of an aromatic compound, which moiety has from 3 to 20 ring atoms. For example, each ring may have from 5 to 8 ring atoms.

In this context, the prefixes (e.g. C₃₋₂₀, C₅₋₈, etc.) denote the number of ring atoms, or range of number of ring atoms, whether carbon atoms or heteroatoms. For example, the term “C₅₋₆ aryl” as used herein, means an aryl group having 5 or 6 ring atoms.

The ring atoms may be all carbon atoms, as in “carboaryl groups”. Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of monocyclic heteroaryl groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: triazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₆).

Examples of heteroaryls which comprise fused rings, include, but are not limited to:

C₉ (with 2 fused rings) derived from benzofuran (O₁), isobenzofuran (O₁), indole (N₁), isoindole (_{NI}), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₂), benzothiofuran (S₁), benzothiazole (N₁S₁), benzothiadiazole (N₂S);

C₁₀ (with 2 fused rings) derived from chromene (O₁), isochromene (O₁), chroman (O₁), isochroman (O₁), benzodioxan (O₂) quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);

C₁₁ (with 2 fused rings) derived from benzodiazepine (N₂);

C₁₃ (with 3 fused rings) derived from carbazole (N₁), dibenzofuran (O₁), dibenzothiophene (S₁), carbolise (N₂), perimidine (N₂), pyridoindole (N₂); and,

C₁₄ (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₂S₁), thianthrene (S₂), phenanthridine (N₁), phenanthroline (N₂), phenazine (N₂).

The above groups, whether alone or part of another substituent, may themselves optionally be substituted with one or more groups selected from themselves and the additional substituents listed below. Further, the substituents listed below may themselves be substituents.

Halo: —F, —Cl, —Br, and —I.

Hydroxy: —OH.

Ether: —OR, wherein R is an ether substituent, for example, a C₁₋₁₀ alkyl group (also referred to as a C₁₋₁₀ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₁₀ alkyl group.

Alkoxy: —OR′, wherein R′ is an alkyl group, for example, a C₁₋₁₀ alkyl group. Examples of C₁₋₁₀ alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Acetal: —CH(OR′₁)(OR′₂), wherein R′₁ and R′₂ are independently acetal substituents, for example, a C₁₋₁₀ alkyl group, a C₃₋₂₀ heterocyclyl group, or a C₅₋₂₀ aryl group, preferably a C₁₋₁₀ alkyl group, or, in the case of a “cyclic” acetal group, R′₁ and R′₂, taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe)(OEt).

Hemiacetal: —CH(OH)(OR′₁), wherein R′₁ is a hemiacetal substituent, for example, a C₁₋₁₀ alkyl group, a C₃₋₂₀ heterocyclyl group, or a C₅₋₂₀ aryl group, preferably a C₁₋₁₀ alkyl group. Examples of hemiacetal groups include, but are not limited to, —CH(OH)(OMe) and —CH(OH)(OEt).

Ketal: —CR′ (OR′₁)(OR′₂), where R′₁ and R′₂ are as defined for acetals, and R′ is a ketal substituent other than hydrogen, for example, a C₁₋₁₀ alkyl group, a C₃₋₂₀ heterocyclyl group, or a

C₅₋₂₀ aryl group, preferably a C₁₋₁₀ alkyl group. Examples ketal groups include, but are not limited to, —C(Me)(OMe)₂, —C(Me)(OEt)₂, —C (Me)(OMe)(OEt), —C(Et)(OMe)₂, —C(Et)(OEt)₂, and —C(Et)(OMe) (OEt).

Hemiketal: —CR′ (OH)(OR′₁), where R′₁ is as defined for hemiacetals, and R′ is a hemiketal substituent other than hydrogen, for example, a C₁₋₁₀ alkyl group, a C₃₋₂₀ heterocyclyl group, or a C₅₋₂₀ aryl group, preferably a C₁₋₁₀ alkyl group. Examples of hemiacetal groups include, but are not limited to, —C(Me)(OH) (OMe), —C (Et)(OH)(OMe), —C (Me)(OH)(OEt), and —C (Et)(OH)(OEt).

Oxo (keto, -one): ═O.

Thione (thioketone): ═S.

Imino (imine): ═NR′, wherein R′ is an imino substituent, for example, hydrogen, a C₁₋₁₀ alkyl group, a C₃₋₂₀ heterocyclyl group, or a C₅₋₂₀ aryl group, preferably a C₁₋₁₀ alkyl group. Examples of ester groups include, but are not limited to, ═NH, ═NMe, =NEt, and ═NPh.

Formyl (carbaldehyde, carboxaldehyde): —C(═O)H.

Acyl (keto): —C(═O)R′, wherein R′ is an acyl substituent, for example, a C₁₋₁₀ alkyl group (also referred to as C₁₋₁₀ alkylacyl or C₁₋₁₀ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₁₀ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR′, wherein R′ is an ester substituent, for example, a C₁₋₁₀ alkyl group, a C₃₋₂₀ heterocyclyl group, or a C₅₋₂₀ aryl group, preferably a C₁₋₁₀ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R′, wherein R′ is an acyloxy substituent, for example, a C₁₋₁₀ alkyl group, a C₃₋₂₀ heterocyclyl group, or a C₅₋₂₀ aryl group, preferably a C₁₋₁₀ alkyl group. Examples of acyloxy groups include, but are not limited to, —OC(═O)CH₃ (acetoxy), —OC(═O)CH₂CH₃, —OC(═O)C(CH₃)₃, —OC(═O) Ph, and —OC(═O)CH₂Ph.

Oxycarboyloxy: —OC(═O)OR, wherein R is an ester substituent, for example, a C₁₋₁₀ alkyl group, a C₃₋₂₀ heterocyclyl group, or a C₅₋₂₀ aryl group, preferably a C₁₋₁₀ alkyl group. Examples of ester groups include, but are not limited to, —OC(═O)OCH₃, —OC(═O)OCH₂CH₃, —OC(═O)OC(CH₃)₃, and —OC(═O)OPh.

Amino: —NR′₁R′₂, wherein R′₁ and R′₂ are independently amino substituents, for example, hydrogen, a C₁₋₁₀ alkyl group (also referred to as C₁₋₁₀ alkylamino or di-C₁₋₁₀ alkylamino), a C₃₋₂₀ heterocyclyl group, or a C₅₋₂₀ aryl group, preferably H or a C₁₋₁₀ alkyl group, or, in the case of a “cyclic” amino group, R′₁ and R′₂, taken together with the nitrogen atom to which they are attached, form a heterocyclic ring having from 4 to 8 ring atoms. Amino groups may be primary (—NH₂), secondary (—NHR′₁), or tertiary (—NHR′₁R′₂), and in cationic form, may be quaternary (—NR′₁R′₂R′₃). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR′₁R′₂, wherein R′₁ and R′₂ are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R′₁ and R′₂, together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S)NR′₁R′₂, wherein R′₁ and R′₂ are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Acylamido (acylamino): —NR′₁C(═O)R′₂, wherein R′₁ is an amide substituent, for example, hydrogen, a C₁₋₁₀ alkyl group, a C₃₋₂₀ heterocyclyl group, or a C₅₋₂₀ aryl group, preferably hydrogen or a C₁₋₁₀ alkyl group, and R′₂ is an acyl substituent, for example, hydrogen, a C₁₋₁₀ alkyl group, a C₃₋₂₀ heterocyclyl group, or a C₅₋₂₀ aryl group, preferably hydrogen or a C₁₋₁₀ alkyl group. Examples of acylamide groups include, but are not limited to, —NHC(═O)CH₃, —NHC(═O)CH₂CH₃, and —NHC(═O)Ph. R′₁ and R′₂ may together form a cyclic structure, as in, for example, succinimidyl, maleimidyl, and phthalimidyl:

Aminocarbonyloxy: —OC(═O)NR′₁R′₂, wherein R′₁ and R′₂ are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.

Ureido: —N(R′₁)C(═O)NR′₂R′₃ wherein R′₂ and R′₃ are independently amino substituents, as defined for amino groups, and R′₁ is a ureido substituent, for example, hydrogen, a C₁₋₁₀ alkyl group, a C₃₋₂₀ heterocyclyl group, or a C₅₋₂₀ aryl group, preferably hydrogen or a C₁₋₁₀ alkyl group. Examples of ureido groups include, but are not limited to, —NHCONH₂, —NHCONHMe, —NHCONHEt, —NHCONMe₂, —NHCONEt₂.

NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom.

Imino: ═NR′, wherein R′ is an imino substituent, for example, for example, hydrogen, a C₁₋₁₀ alkyl group, a C₃₋₂₀ heterocyclyl group, or a C₅₋₂₀ aryl group, preferably hydrogen or a C₁₋₁₀ alkyl group. Examples of imino groups include, but are not limited to, ═NH, ═NMe, and =NEt.

Amidine (amidino): —C(═NR′₁)NR′₂, wherein each is an amidine substituent, for example, hydrogen, a C₁₋₁₀ alkyl group, a C₃₋₂₀ heterocyclyl group, or a C₅₋₂₀ aryl group, preferably hydrogen or a C₁₋₁₀ alkyl group. Examples of amidine groups include, but are not limited to, —C(═NR′₁)NH₂, —C(═NH)NMe₂, and —C(═NMe)NMe₂.

Nitro: —NO2.

Nitroso: —NO.

Azido: —N3.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR′, wherein R′ is a thioether substituent, for example, a C₁₋₁₀ alkyl group (also referred to as a C₁₋₁₀ alkylthio group), a C₃₋₂₀ heterocyclyl group, or a C₅₋₂₀ aryl group, preferably a C₁₋₁₀ alkyl group. Examples of C₁₋₁₀ alkylthio groups include, but are not limited to, —SCH₃ and —SCH₂CH₃.

Disulfide: —SS—R′, wherein R′ is a disulfide substituent, for example, a C₁₋₁₀ alkyl group, a C₃₋₂₀ heterocyclyl group, or a C₅₋₂₀ aryl group, preferably a C₁₋₁₀ alkyl group (also referred to herein as C₁₋₁₀ alkyl disulfide). Examples of C₁₋₁₀ alkyl disulfide groups include, but are not limited to, —SSCH₃ and —SSCH₂CH₃.

Sulfine (sulfinyl, sulfoxide): —S(═O)R′, wherein R′ is a sulfine substituent, for example, a C₁₋₁₀ alkyl group, a C₃₋₂₀ heterocyclyl group, or a C₅₋₂₀ aryl group, preferably a C₁₋₁₀ alkyl group. Examples of sulfine groups include, but are not limited to, —S(═O)CH₃ and —S(═O)CH₂CH₃.

Sulfone (sulfonyl): —S(═O)₂R′, wherein R′ is a sulfone substituent, for example, a C₁₋₁₀ alkyl group, a C₃₋₂₀ heterocyclyl group, or a C₅₋₂₀ aryl group, preferably a C₁₋₁₀ alkyl group, including, for example, a fluorinated or perfluorinated C₁₋₁₀ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S (═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR′; wherein R′ is a sulfinate substituent, for example, a C₁₋₁₀ alkyl group, a C₃₋₂₀ heterocyclyl group, or a C₅₋₂₀ aryl group, preferably a C₁₋₁₀ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR′, wherein R′ is a sulfonate substituent, for example, a C₁₋₁₀ alkyl group, a C₃₋₂₀ heterocyclyl group, or a C₅₋₂₀ aryl group, preferably a C₁₋₁₀ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R′, wherein R is a sulfinyloxy substituent, for example, a C₁₋₁₀ alkyl group, a C₃₋₂₀ heterocyclyl group, or a C₅₋₂₀ aryl group, preferably a C₁₋₁₀ alkyl group. Examples of sulfinyloxy groups include, but are not limited to, —OS(═O)CH₃ and —OS(═O)CH₂CH₃.

Sulfonyloxy: —OS(═O)₂R′, wherein R′ is a sulfonyloxy substituent, for example, a C₁₋₁₀ alkyl group, a C₃₋₂₀ heterocyclyl group, or a C₅₋₂₀ aryl group, preferably a C₁₋₁₀ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR′; wherein R′ is a sulfate substituent, for example, a C₁₋₁₀ alkyl group, a C₃₋₂₀ heterocyclyl group, or a C₅₋₂₀ aryl group, preferably a C₁₋₁₀ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR′₁R′₂, wherein R′₁ and R′₂ are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR′₁R′₂, wherein R′₁ and R′₂ are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR'S(═O)₂OH, wherein R′ is an amino substituent, as defined for amino groups. Examples of sulfamino groups include, but are not limited to, —NHS(═O)₂OH and —N(CH₃)S(═O)₂OH.

Sulfonamido: —NR′₁S(═O)₂R′₂, wherein R′₁ is an amino substituent, as defined for amino groups, and R′₂ is a sulfonamino substituent, for example, a C₁₋₁₀ alkyl group, a C₃₋₂₀ heterocyclyl group, or a C₅₋₂₀ aryl group, preferably a C₁₋₁₀ alkyl group. Examples of sulfonamino groups include, but are not limited to, —NHS(═O)₂CH₃ and —N(CH₃)S(═O)₂C₆H₅.

Phosphino (phosphine): —P(R′)₂, wherein R′ is a phosphino substituent, for example, a C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphino groups include, but are not limited to, —PH₂, —P(CH₃)₂, —P(CH₂CH₃)₂, —P(t-Bu)₂, and —P(Ph)₂-Phospho: —P(═O)₂.

Phosphinyl (phosphine oxide): —P(═O)(R′)₂, wherein R′ is a phosphinyl substituent, for example, a C₁₋₁₀ alkyl group, a C₃₋₂₀ heterocyclyl group, or a C₅₋₂₀ aryl group, preferably a C₁₋₁₀ alkyl group or a C₅₋₂₀ aryl group. Examples of phosphinyl groups include, but are not limited to, —P(═O)(CH₃)₂, —P(═O)(CH₂CH₃)₂, —P(═O)(t-Bu)₂, and —P(═O)(Ph)₂.

Phosphonic acid (phosphono): —P(═O)(OH)₂.

Phosphonate (phosphono ester): —P(═O)(OR′)₂, where R′ is a phosphonate substituent, for example, hydrogen, a C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphonate groups include, but are not limited to, P(═O)(OCH₃)₂, —P(═O)(OCH₂CH₃)₂, —P(═O)(O-t-Bu)₂, and —P(═O)(OPh)₂.

Phosphoric acid (phosphonooxy): —OP(═O)(OH)₂.

Phosphate (phosphonooxy ester): —OP(═O)(OR′)₂, where R′ is a phosphate substituent, for example, hydrogen, a C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphate groups include, but are not limited to, —OP(═O)(OCH₃)₂, —OP(═O)(OCH₂CH₃)₂, —OP(═O)(O-t-Bu)₂, and —OP(═O)(OPh)₂.

Phosphorous acid: —OP(OH)₂.

Phosphite: —OP(OR′)₂, where R′ is a phosphite substituent, for example, hydrogen, a C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphite groups include, but are not limited to, —OP(OCH₃)₂, —OP(OCH₂CH₃)₂, —OP(O-t-Bu)₂, and —OP(OPh)₂.

Phosphoramidite: —OP(OR′₁)—N(R′₂)₂, where R′₁ and R′₂ are phosphoramidite substituents, for example, hydrogen, a (optionally substituted) C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphoramidite groups include, but are not limited to, —OP(OCH₂CH₃)—N(CH₃)₂, —OP(OCH₂CH₃)—N (i-Pr)₂, and —OP(OCH₂CH₂CN)—N(i-Pr)₂.

Phosphoramidate: —OP(═O)(OR′₁)—N(R′₂)₂, where R′₁ and R′₂ are phosphoramidate substituents, for example, hydrogen, a (optionally substituted) C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N (i-Pr)₂, and —OP(═O)(OCH₂CH₂CN)—N (i-Pr)₂.

In an embodiment, the term “alkylene” means a bidentate moiety obtained by removing two hydrogen atoms, either both from the same carbon atom, or one from each of two different carbon atoms, of a hydrocarbon compound, which may be aliphatic or alicyclic, and which may be saturated, partially unsaturated, or fully unsaturated. Thus, the term “alkylene” includes the sub-classes alkenylene, alkynylene, cycloalkylene, etc., discussed below.

Examples of linear saturated C₃₋₁₂ alkylene groups include, but are not limited to, —(CH₂)ₙ— where n is an integer from 3 to 12, for example, —CH₂CH₂CH₂— (propylene), —CH₂CH₂CH₂CH₂-(butylene), —CH₂CH₂CH₂CH₂CH₂— (pentylene) and —CH₂CH₂CH₂CH₂CH₂CH₂CH₂— (heptylene).

Examples of branched saturated C₃₋₁₂ alkylene groups include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₃₋₁₂ alkylene groups (C₃₋₁₂ alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated C₃₋₁₂ alkylene groups (C₃₋₁₂ alkenylene and alkynylene groups) include, but are not limited to, —C(CH₃)═CH—, —C(CH₃)═CH—CH₂—, —CH═CH—CH(CH₃)— and —C≡C—CH(CH₃)—.

Examples of alicyclic saturated C₃₋₁₂ alkylene groups (C₃₋₁₂ cycloalkylenes) include, but are not limited to, cyclopentylene (e.g. cyclopent-1,3-ylene), and cyclohexylene (e.g. cyclohex-1,4-ylene).

Examples of alicyclic partially unsaturated C₃₋₁₂ alkylene groups (C₃₋₁₂ cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

In an embodiment, the term “glycoside” means a carbohydrate or glycan moiety that is joined by a glycosidic bond. The glycosidic bond may be an O-, N-, C- or S-glycosidic bond, meaning that the bond is formed to the anomeric carbon of the glycan moiety by an oxygen, nitrogen, carbon or sulphur atom, respectively. The glycosidic bond may be an acetal bond. The glycan may be any monosaccharide, disaccharide, oligosaccharide or polysaccharide, and it may be further substituted by any of the substituents listed above.

Examples of glycoside groups include, but are not limited to, β-D-O-galactoside, N-acetyl-β-D-O-galactosaminide, N-acetyl-α-D-O-galactosaminide, N-acetyl-β-D-O-glucosaminide, N-acetyl-β-D-N-glucosaminide, β-D-O-glucuronide, α-L-O-iduronide, α-D-O-galactoside, α-D-O-glucoside, α-D-C-glucoside, β-D-O-glucoside, α-D-O-mannoside, β-D-O-mannoside, β-D-C-mannoside, α-L-O-fucoside, β-D-O-xyloside, N-acetyl-α-D-O-neuraminide, lactoside, maltoside, dextran, and any analogue or modification thereof.

In an embodiment, an anomeric bond of a glycan moiety may be represented by a wavy line, which indicates that the stereochemistry of the anomeric carbon is not defined and it may exist in either the R or S configuration, in other words beta or alpha configuration, meaning that when the glycan is drawn as a ring the bond may be directed either above or below the ring. In a further embodiment, if the anomeric carbon is drawn with a wavy bond to a hydroxyl group (thus forming a hemiacetal) the wavy bond indicates that the glycan can also exist in the open-ring form (aldehyde or ketone).

In an embodiment, the term “polyethylene glycol” means a polymer comprising repeating “PEG” units of the formula [CH₂CH₂O]ₙ. In an embodiment, the term “PEG₁₋₅₀” means polyethylene glycol moiety having from 1 to 50 PEG units. In an embodiment, the term “substituted polyethylene glycol” means a polyethylene glycol substituted with one or more of the substituents listed above. In an embodiment, the term “branched polyethylene glycol” means a polyethylene glycol moiety substituted with one or more of polyethylene glycol substituents forming a branched structure.

The conjugate may be represented by formula I:

[D-L]ₙ-T Formula I

wherein D is the glycosylation inhibitor, T is the targeting unit, L is a linker unit linking D to T at least partially covalently, and n is at least 1.

In formula I, when n is greater than 1, each D may, in principle, be selected independently. Each L may likewise be selected independently.

In formula I, n may be an integer, for example an integer of at least 1.

In formula I, n may be in the range of 1 to about 20, or 1 to about 15, or 1 to about 10, or 2 to 10, or 2 to 6, or 2 to 5, or 2 to 4, or 3 to about 20, or 3 to about 15, or 3 to about 10, or 3 to about 9, or 3 to about 8, or 3 to about 7, or 3 to about 6, or 3 to 5, or 3 to 4, or 4 to about 20, or 4 to about 15, or 4 to about 10, or 4 to about 9, or 4 to about 8, or 4 to about 7, or 4 to about 6, or 4 to 5; or about 7-9; or about 8, or 1, 2, 3, 4, 5, 6, 7, 8, 9, 10, 11, 12, 13, 14, 15, 16, 17, 18, 19, or 20; or in the range of 1 to about 1000, or 1 to about 2000, or 1 to about 400, or 1 to about 200, or 1 to about 100; or 100 to about 1000, or 200 to about 1000, or 400 to about 1000, or 600 to about 1000, or 800 to about 1000; 100 to about 800, or 200 to about 600, or 300 to about 500; or 20 to about 200, or 30 to about 150, or 40 to about 120, or 60 to about 100; over 8, over 16, over 20, over 40, over 60, over 80, over 100, over 120, over 150, over 200, over 300, over 400, over 500, over 600, over 800, or over 1000; or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 52, 54, 56, 58, 60, 62, 63, 64, 66, 68, 70, 72, 74, 76, 78, 80, 82, 84, 86, 88, 90, 92, 94, 96, 98, 100, 110, 120, 130, 140, 150, 160, 170, 180, 190, 200, 220, 240, 260, 280, 300, 350, 400, 450, 500, 550, 600, 650, 700, 750, 800, 850, 900, 950, 1000, 1100, 1200, 1300, 1400, 1500, 1600, 1700, 1800, 1900, or 2000, or greater than 2000.

II) Glycosylation Inhibitors

In an embodiment, the glycosylation inhibitor is a glycosylation inhibitor described in any one of the following publications: Esko et al. 2017, in Essentials of Glycobiology, 3^{rd} edition, Chapter 55; Chapman et al. 2004, Angew Chem Int Ed Engl 43:3526-48; Dorfmueller et al. 2006, J Am Chem Soc 128:16484-5; Brown et al. 2007, Crit Rev Biochem Mol Biol 42:481-515; Chaudhary et al. 2013, Mini Rev Med Chem 13:222-36; Tu et al. 2013. Chem Soc Rev 42:4459-75; Galley et al. 2014, Bioorg Chem 55:16-26; Gouin 2014, Chemistry 20:11616-28; Kallemeijn et al. 2014, Adv Carbohydr Chem Biochem 71:297-338; Kim et al. 2014, Crit Rev Biochem Mol Biol 49:327-42. Shayman & Larsen 2014, J Lipid Res 55:1215-25.

In an embodiment, the glycosylation inhibitor is a hydrophilic glycosylation inhibitor, such as a nonacetylated saccharide analog. The hydrophilicity may have the benefit that the hydrophilic glycosylation inhibitor may have a poor ability to enter non-target cells if it is prematurely released from the conjugate before reaching the target tissue such as the tumour or the target cell. For example, UDP-GlcNAc levels do not necessarily change significantly in response to unacetylated 4-fluoro-GlcNAc treatment, from the outside of the cell, of either human leukemia cell line KG1a or T cells, whereas treatment with peracetylated 4-fluoro-GlcNAc may significantly decrease UDP-GlcNAc levels in these cells and thereby may be capable of effectively inhibiting glycosylation in any cell, without discriminating between different cell types (Barthel et al. 2011, J. Biol. Chem. 286:21717-31). Hydrophilic glycosylation inhibitors may also be substantially non-toxic.

In an embodiment, the glycosylation inhibitor is a hydrophobic glycosylation inhibitor, such as a peracetylated saccharide analog. The hydrophobicity may have the benefit that the hydrophobic glycosylation inhibitor may have a good ability to enter target cells if prematurely released from the conjugate after reaching the target tissue such as tumour, but before reaching the target cell. Moreover, the hydrophobic glycosylation inhibitor may have a good ability to enter another target cell or the second tumour cell after inhibiting glycosylation in the (first) target cell.

In an embodiment, the glycosylation inhibitor is selected from the groups of:
1) Metabolic inhibitors, which are capable of interfering with steps involved in formation of common intermediates of a glycosylation pathway, such as nucleotide sugars;
2) Cellular trafficking inhibitors, which are capable of impeding the structure of or transit between the endoplasmic reticulum (ER), Golgi, and/or trans-Golgi network;
3) Tunicamycin, which is capable of inhibiting N-linked glycosylation through inhibition of dolichol-PP-GlcNAc formation and peptidoglycan biosynthesis through inhibition of undecaprenyl-PP-GlcNAc assembly;
4) Plant alkaloids, which are capable of inhibiting N-linked glycosylation through inhibition of processing glycosidases;
5) Substrate analogs, which are capable of inhibiting specific glycosyltransferases or glycosidases;
6) Glycoside primers, which are capable of inhibiting glycosylation pathways by diverting the assembly of glycans from endogenous acceptors to exogenous primers; and
7) Specific inhibitors of glycosylation, which may include, for example, interfering RNA to specific glycosyltransferases, and the like.

In an embodiment, the glycosylation inhibitor is selected from the groups 1)-7) above and any analogs or modifications thereof.

In an embodiment, the glycosylation inhibitor comprises or is a metabolic inhibitor (group 1).

In an embodiment, the glycosylation inhibitor comprises or is a cellular trafficking inhibitor (group 2).

In an embodiment, the glycosylation inhibitor comprises or is a tunicamycin (group 3).

In an embodiment, the glycosylation inhibitor comprises or is a plant alkaloid (group 4).

In an embodiment, the glycosylation inhibitor comprises or is a substrate analog (group 5). Such substrate analog may be capable of inhibiting a specific glycosyltransferase and/or glycosidase.

In an embodiment, the glycosylation inhibitor comprises or is a glycoside primer (group 6).

In an embodiment, the glycosylation inhibitor comprises or is a specific inhibitor (group 7).

In an embodiment, the glycosylation inhibitor comprises or is a metabolic inhibitor (group 1); a cellular trafficking inhibitor (group 2); a tunicamycin (group 3); a plant alkaloid (group 4); a substrate analog (group 5); a glycoside primer (group 6); and/or a specific inhibitor (group 7).

The glycosylation inhibitor may be selected from the group of a metabolic inhibitor, a cellular trafficking inhibitor, tunicamycin, a plant alkaloid, a substrate analog, a glycoside primer, a specific inhibitor of glycosylation, an N-acetylglucosaminylation inhibitor, an N-acetylgalactosaminylation inhibitor, a sialylation inhibitor, a fucosylation inhibitor, a galactosylation inhibitor, a xylosylation inhibitor, a glucuronylation inhibitor, a mannosylation inhibitor, a mannosidase inhibitor, a glucosidase inhibitor, a glucosylation inhibitor, an N-glycosylation inhibitor, an O-glycosylation inhibitor, a glycosaminoglycan biosynthesis inhibitor, a glycosphingolipid biosynthesis inhibitor, a sulphation inhibitor, Brefeldin A, 6-diazo-5-oxo-L-norleucine, chlorate, 2-deoxyglucose, a fluorinated sugar analog, 2-acetamido-2,4-dideoxy-4-fluoroglucosamine, 2-acetamido-2,3-dideoxy-3-fluoroglucosamine, 2-acetamido-2,6-dideoxy-6-fluoroglucosamine, 2-acetamido-2,5-dideoxy-5-fluoroglucosamine, 4-deoxy-4-fluoroglucosamine, 3-deoxy-3-fluoroglucosamine, 6-deoxy-6-fluoroglucosamine, 5-deoxy-5-fluoroglucosamine, 3-deoxy-3-fluoro-sialic acid, 3-deoxy-3ax-fluorosialic acid, 3-deoxy-3eq-fluoro-sialic acid, 3-deoxy-3-fluoro-Neu5Ac, 3-deoxy-3ax-fluoro-Neu5Ac, 3-deoxy-3eq-fluoro-Neu5Ac, 3-deoxy-3-fluorofucose, 2-deoxy-2-fluoroglucose, 2-deoxy-2-fluoromannose, 2-deoxy-2-fluorofucose, 3-fluoro-sialic acid, castanospermine, australine, deoxynojirimycin, N-butyldeoxynojirimycin, deoxymannojirimycin, kifunensin, swainsonine, mannostatin A, alloxan, streptozotocin, 2-acetamido-2,5-dideoxy-5-thioglucosamine, 2-acetamido-2,4-dideoxy-4-thioglucosamine, PUGNAc (0-[2-acetamido-2-deoxy-D-glucopyranosylidene]amino-N-phenylcarbamate), Thiamet-G, N-acetylglucosamine-thiazoline (NAG-thiazoline), GlcNAcstatin, a nucleotide sugar analog, a UDP-GlcNAc analog, a UDP-GalNAc analog, a UDP-Glc analog, a UDP-Gal analog, a GDP-Man analog, a GDP-Fuc analog, a UDP-GlcA analog, a UDP-Xyl analog, a CMP-Neu5Ac analog, a nucleotide sugar bisubstrate, a glycoside primer, a β-xyloside, a β-N-acetylgalactosaminide, a β-glucoside, a β-galactoside, β-N-acetylglucosaminide, a β-N-acetyllactosaminide, a disaccharide glycoside and a trisaccharides glycoside, 4-methyl-umbelliferone, glucosylceramide epoxide, D-threo-1-phenyl-2-decanoylamino-3-morpholino-1-propanol (PDMP), PPPP, 2-amino-2-deoxymannose, a 2-acyl-2-deoxy-glucosyl-phosphatidylinositol, 10-propoxydecanoic acid, Neu5Ac-2-ene (DANA), 4-amino-DANA, 4-guanidino-DANA, (3R, 4R, 5S)-4-acetamido-5-amino-3-(1-ethylpropoxyl)-1-cyclohexane-1-carboxylic acid, (3R, 4R, 5S)-4-acetamido-5-amino-3-(1-ethylpropoxyl)-1-cyclohexane-1-carboxylic acid ethyl ester, 2,6-dichloro-4-nitrophenol, pentachlorophenol, a mannosidase I inhibitor, a glucosidase I inhibitor, a glucosidase II inhibitor, an N-acetylglucosaminyltransferase inhibitor, an N-acetylgalactosaminyltransferase inhibitor, a galactosyltransferase inhibitor, a sialyltransferase inhibitor, a hexosamine pathway inhibitor, a glutamine-fructose-6-phosphate aminotransferase (GFPT1) inhibitor, a phosphoacetylglucosamine mutase (PGM3) inhibitor, a UDP-GlcNAc synthase inhibitor, a CMP-sialic acid synthase inhibitor, N-acetyl-D-glucosamine-oxazoline, 6-methyl-phosphonate-N-acetyl-D-glucosamine-oxazoline, 6-methyl-phosphonate-N-acetyl-D-glucosamine-thiazoline, V-ATPase inhibitor, a concanamycin, concanamycin A, concanamycin B, concanamycin C, a bafilomycin, bafilomycin A1, an archazolid, archazolid A, a salicylihalamide, salicylihalamide A, an oximidine, oximidine I, a lobatamide, lobatamide A, an apicularen, apicularen A, apicularen B, cruentaren, a plecomacrolide, (2Z,4E)-5-(5,6-dichloro-2-indolyl)-2-methoxy-N-(1,2,2,6,6-pentamethylpiperidin-4-yl)-2,4-pentadienamide (INDOLO), epi-kifunensine, deoxyfuconojirimycin, 1,4-dideoxy-1,4-imino-D-mannitol, 2,5-dideoxy-2,5-imino-D-mannitol, 1,4-dideoxy-1,4-imino-D-xylitol, a lysophospholipid acyltransferase (LPAT) inhibitor, a cytoplasmic phospholipase A₂ (PLA₂) inhibitor, an acyl-CoA cholesterol acyltransferase (ACAT) inhibitor, CI-976, an N-acyldeoxynojirimycin, N-acetyldeoxynojirimycin, an N-acyldeoxymannojirimycin, N-acetyldeoxymannojirimycin, a coat protein (COPI) inhibitor, a brefeldin, tamoxifen, raloxifene, sulindac, 3-deoxy-3-fluoro-Neu5N, 3-deoxy-3ax-fluoro-Neu5N, 3-deoxy-3eq-fluoro-Neu5N, 3′-azido-3′-deoxythymidine, 3′-fluoro-3′-deoxythymidine, 3′-azido-3′-deoxycytidine, 3′-fluoro-3′-deoxycytidine, 3′-azido-2′,3′-dideoxycytidine, 3′-fluoro-2′,3′-dideoxycytidine, and any analogs, modifications, acylated analogs, acetylated analogs, methylated analogs, or combinations thereof.

The glycosylation inhibitor may, in an embodiment, be selected from the group of 3′-azido-3′-deoxythymidine, 3′-fluoro-3′-deoxythymidine, 3′-azido-3′-deoxycytidine, 3′-fluoro-3′-deoxycytidine, 3′-azido-2′,3′-dideoxycytidine, and 3′-fluoro-2′,3′-dideoxycytidine.

In an embodiment, the metabolic inhibitor (group 1) is selected from the group of a sulphation inhibitor, chlorate, 2-deoxyglucose, D-threo-1-phenyl-2-decanoylamino-3-morpholino-1-propanol (PDMP), DL-threo-phenyl-2-hexadecanoylamino-3-pyrrolidino-1-propanol (PPPP), 2-amino-2-deoxymannose, a 2-acyl-2-deoxy-glucosyl-phosphatidylinositol, 10-propoxydecanoic acid, 2,6-dichloro-4-nitrophenol, pentachlorophenol, a hexosamine pathway inhibitor, a glutamine-fructose-6-phosphate aminotransferase (GFPT1) inhibitor, a phosphoacetylglucosamine mutase (PGM3) inhibitor, a UDP-GlcNAc synthase inhibitor, a CMP-sialic acid synthase inhibitor, a glycosaminoglycan biosynthesis inhibitor, a glycosphingolipid biosynthesis inhibitor, and any analogs, modifications, acylated analogs, acetylated analogs, methylated analogs, or combinations thereof.

In an embodiment, the cellular trafficking inhibitor (group 2) is selected from the group of a coat protein (COPI) inhibitor, a brefeldin, Brefeldin A, V-ATPase inhibitor, a concanamycin, concanamycin A, concanamycin B, concanamycin C, a bafilomycin, bafilomycin A1, an archazolid, archazolid A, a salicylihalamide, salicylihalamide A, an oximidine, oximidine I, a lobatamide, lobatamide A, an apicularen, apicularen A, apicularen B, cruentaren, a plecomacrolide, (2Z,4E)-5-(5,6-dichloro-2-indolyl)-2-methoxy-N-(1,2,2,6,6-pentamethylpiperidin-4-yl)-2,4-pentadienamide (INDOLO), a lysophospholipid acyltransferase (LPAT) inhibitor, a cytoplasmic phospholipase A₂ (PLA₂) inhibitor, an acyl-CoA cholesterol acyltransferase (ACAT) inhibitor, CI-976, and any analogs, modifications, acylated analogs, acetylated analogs, methylated analogs, or combinations thereof.

In an embodiment, the tunicamycin (group 3) is selected from the group of tunicamycin and any analogs, modifications, acylated analogs, acetylated analogs, methylated analogs, or combinations thereof.

In an embodiment, the plant alkaloid (group 4) is selected from the group of an N-acyldeoxynojirimycin, N-acetyldeoxynojirimycin, an N-acyldeoxymannojirimycin, N-acetyldeoxymannojirimycin, epi-kifunensine, deoxyfuconojirimycin, 1,4-dideoxy-1,4-imino-D-mannitol, 2,5-dideoxy-2,5-imino-D-mannitol, 1,4-dideoxy-1,4-imino-D-xylitol, castanospermine, australine, deoxynojirimycin, N-butyldeoxynojirimycin, deoxymannojirimycin, kifunensin, swainsonine, mannostatin A, and any analogs, modifications, acylated analogs, acetylated analogs, methylated analogs, or combinations thereof.

In an embodiment, the substrate analog (group 5) is selected from the group of a fluorinated sugar analog, 2-acetamido-2,4-dideoxy-4-fluoroglucosamine, 2-acetamido-2,3-dideoxy-3-fluoroglucosamine, 2-acetamido-2,6-dideoxy-6-fluoroglucosamine, 2-acetamido-2,5-dideoxy-5-fluoroglucosamine, 4-deoxy-4-fluoroglucosamine, 3-deoxy-3-fluoroglucosamine, 6-deoxy-6-fluoroglucosamine, 5-deoxy-5-fluoroglucosamine, 3-deoxy-3-fluoro-sialic acid, 3-deoxy-3ax-fluorosialic acid, 3-deoxy-3eq-fluorosialic acid, 3-deoxy-3-fluoro-Neu5Ac, 3-deoxy-3ax-fluoro-Neu5Ac, 3-deoxy-3eq-fluoro-Neu5Ac, 3-deoxy-3-fluorofucose, 2-deoxy-2-fluoroglucose, 2-deoxy-2-fluoromannose, 2-deoxy-2-fluorofucose, 3-fluorosialic acid, alloxan, streptozotocin, 2-acetamido-2,5-dideoxy-5-thioglucosamine, 2-acetamido-2,4-dideoxy-4-thioglucosamine, PUGNAc (0-[2-acetamido-2-deoxy-D-glucopyranosylidene]amino-N-phenylcarbamate), Thiamet-G, N-acetylglucosamine-thiazoline (NAG-thiazoline), GlcNAcstatin, a nucleotide sugar analog, a UDP-GlcNAc analog, a UDP-GalNAc analog, a UDP-Glc analog, a UDP-Gal analog, a GDP-Man analog, a GDP-Fuc analog, a UDP-GlcA analog, a UDP-Xyl analog, a CMP-Neu5Ac analog, a nucleotide sugar bisubstrate, Neu5Ac-2-ene (DANA), 4-amino-DANA, 4-guanidino-DANA, (3R, 4R, 5S)-4-acetamido-5-amino-3-(1-ethylpropoxyl)-1-cyclohexane-1-carboxylic acid, (3R, 4R, 5S)-4-acetamido-5-amino-3-(1-ethylpropoxyl)-1-cyclohexane-1-carboxylic acid ethyl ester, N-acetyl-D-glucosamine-oxazoline, 6-methylphosphonate-N-acetyl-D-glucosamine-oxazoline, 6-methylphosphonate-N-acetyl-D-glucosamine-thiazoline, 3-deoxy-3-fluoro-Neu5N, 3-deoxy-3ax-fluoro-Neu5N, 3-deoxy-3eq-fluoro-Neu5N, and any analogs, modifications, acylated analogs, acetylated analogs, methylated analogs, or combinations thereof.

In an embodiment, the glycoside primer (group 6) is selected from the group of a glycoside primer, a β-xyloside, a β-N-acetylgalactosaminide, a β-glucoside, a β-galactoside, β-N-acetylglucosaminide, a β-N-acetyllactosaminide, a disaccharide glycoside and a trisaccharides glycoside, 4-methyl-umbelliferone, glucosylceramide epoxide, and any analogs, modifications, acylated analogs, acetylated analogs, methylated analogs, or combinations thereof.

In an embodiment, the specific inhibitor of glycosylation (group 7) is selected from the group of an N-acetylglucosaminylation inhibitor, an N-acetylgalactosaminylation inhibitor, a sialylation inhibitor, a fucosylation inhibitor, a galactosylation inhibitor, a xylosylation inhibitor, a glucuronylation inhibitor, a mannosylation inhibitor, a mannosidase inhibitor, a glucosidase inhibitor, a glucosylation inhibitor, an N-glycosylation inhibitor, an O-glycosylation inhibitor, a mannosidase I inhibitor, a glucosidase I inhibitor, a glucosidase II inhibitor, an N-acetylglucosaminyltransferase inhibitor, an N-acetylgalactosaminyltransferase inhibitor, a galactosyltransferase inhibitor, a sialyltransferase inhibitor, 6-diazo-5-oxo-L-norleucine, tamoxifen, raloxifene, sulindac and any analogs, modifications, acylated analogs, acetylated analogs, methylated analogs, or combinations thereof.

In an embodiment, the N-glycosylation inhibitor is selected from the group of a tunicamycin, a tunicamycin analog, a UDP-N-acetylglucosamine: dolichyl-phosphate N-acetylglucosamine-phosphotransferase (GlcNAc-1-β-transferase) inhibitor, an oligosaccharyltransferase inhibitor, an N-glycan precursor synthesis inhibitor and an N-glycan processing inhibitor.

In an embodiment, the N-glycan processing inhibitor is selected from the group of a glucosidase inhibitor, a glucosidase I inhibitor, a glucosidase II inhibitor, a mannosidase inhibitor, a mannosidase I inhibitor, a mannosidase II inhibitor and an N-acetyl-glucosaminyltransferase inhibitor.

In an embodiment, the N-acetylglucosaminylation inhibitor is selected from the group of 2-acetamido-2,4-dideoxy-4-fluoroglucosamine, 2-acetamido-2,3-dideoxy-3-fluoroglucosamine, 2-acetamido-2,6-dideoxy-6-fluoroglucosamine, 2-acetamido-2,5-dideoxy-5-fluoroglucosamine, 4-deoxy-4-fluoroglucosamine, 3-deoxy-3-fluoroglucosamine, 6-deoxy-6-fluoroglucosamine, 5-deoxy-5-fluoroglucosamine, a UDP-GlcNAc analog, a hexosamine pathway inhibitor, and any analogs or modifications thereof.

In an embodiment, the sialylation inhibitor is selected from the group of 3-deoxy-3-fluorosialic acid, 3-deoxy-3ax-fluoro-sialic acid, 3-deoxy-3eq-fluorosialic acid, 3-deoxy-3-fluoro-Neu5Ac, 3-deoxy-3ax-fluoro-Neu5Ac, 3-deoxy-3eq-fluoro-Neu5Ac, 3-fluoro-sialic acid, a CMP-Neu5Ac analog, a β-N-acetyllactosaminide, Neu5Ac-2-ene (DANA), 4-amino-DANA, 4-guanidino-DANA, (3R, 4R, 5S)-4-acetamido-5-amino-3-(1-ethylpropoxyl)-1-cyclohexane-1-carboxylic acid, (3R, 4R, 5S)-4-acetamido-5-amino-3-(1-ethylpropoxyl)-1-cyclohexane-1-carboxylic acid ethyl ester, a sialyltransferase inhibitor, a CMP-sialic acid synthase inhibitor, 3-deoxy-3-fluoro-Neu5N, 3-deoxy-3ax-fluoro-Neu5N, 3-deoxy-3eq-fluoro-Neu5N, a hexosamine pathway inhibitor, and any analogs or modifications thereof.

In an embodiment, the galactosylation inhibitor is selected from the group of a galactosyltransferase inhibitor, a UDP-Gal analog, galactosyltransferase inhibitor, and any analogs or modifications thereof.

In an embodiment, the hexosamine pathway inhibitor is selected from the group of a glutamine-fructose-6-phosphate aminotransferase (GFPT1) inhibitor, a phosphoacetylglucosamine mutase (PGM3) inhibitor, a UDP-GlcNAc synthase inhibitor, N-acetyl-D-glucosamine-oxazoline, 6-methyl-phosphonate-N-acetyl-D-glucosamine-oxazoline, 6-methyl-phosphonate-N-acetyl-D-glucosamine-thiazoline, 6-diazo-5-oxo-L-norleucine, and any analogs, homologs or modifications thereof.

In an embodiment, the tunicamycin is selected from the group of tunicamycin I, tunicamycin II, tunicamycin III, tunicamycin IV, tunicamycin V, tunicamycin VI, tunicamycin VII, tunicamycin VIII, tunicamycin IX and tunicamycin X, and tunicamycins A, A0, A1, A2, A3, A4, B, B1, B2, B3, B4, B5, B6, C, C1, C2, C3, D, D1, D2, Tun 16:0A, Tun 16:0B, Tun 17:2, Tun 17:0A, Tun 17:0B, Tun 17:0C, Tun 18:1A and Tun 18:1B, and as described in Ito et al. 1980 (Agric. Biol. Chem. 44:695-8) and references therein and in Tsvetanova & Price 2001 (Anal. Biochem. 289:147-56) and references therein, and any analogs, homologs or modifications thereof. In an embodiment, the glucosidase inhibitor is selected from the group of a glucosidase I inhibitor, a glucosidase II inhibitor, and a combination thereof.

In an embodiment, the glucosidase inhibitor is selected from the group of australine, epi-kifunensine, 1-deoxynojirimycin, an N-acyldeoxynojirimycin, N-acetyldeoxynojirimycin, and any analogs, combinations or modifications thereof.

In an embodiment, the mannosidase inhibitor is selected from the group of a mannosidase I inhibitor, a mannosidase II inhibitor, a lysosomal mannosidase inhibitor and a combination thereof.

In an embodiment, the mannosidase inhibitor is a combination of a mannosidase I inhibitor and a mannosidase II inhibitor. In an embodiment, the mannosidase inhibitor is a combination of kifunensine and swainsonine.

In an embodiment, the mannosidase I inhibitor is selected from the group of kifunensine, 1-deoxymannojirimycin, N-acyl-1-deoxymannojirimycin, N-acetyl-1-deoxymannojirimycin, N-alkyl-1-deoxymannojirimycin, N-butyl-1-deoxymannojirimycin, tamoxifen, raloxifene, sulindac, and any analogs or modifications thereof.

In an embodiment, the mannosidase II inhibitor is selected from the group of swainsonine, mannostatin A, and any analogs or modifications thereof.

The glycosylation inhibitor may be represented by formula II:

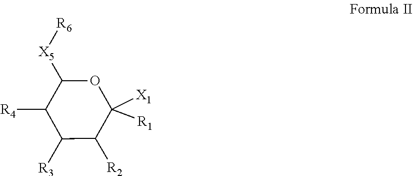

wherein X₁ is H, COOH, COOCH₃ or COOL′;

R₁ is absent, OH, OZ or L′;

R₂ is absent, Y, OH, OZ, NHCOCH₃ or L′;

R₃ is absent, Y, OH, OZ or L′;

R₄ is absent, Y, OH, OZ, NHCOCH₃ or L′;

X₅ is absent, CH₂, CH(OH)CH₂, CH(OZ)CH₂, CH(OH)CH(OH)CH₂, CH(OZ)CH(OZ)CH₂, a C₁-C₁₂ alkyl, or a substituted C₁-C₁₂ alkyl;

R₆ is absent, Y, OH, OZ or L′;

L′ is a bond to L;

each Z is independently selected from COCH₃, a C₁-C₁₂ acyl and a substituted C₁-C₁₂ acyl; and

Y is selected from F, Cl, Br, I, H and CH₃;

with the proviso that not more than one of R₁, R₂, R₃, R₄ and R₆ is Y, and that D contains not more than one L′.

The phrase “R₁ (or R₂, R₃, R₄, X₅, R₆, or any other substituent or radical described in this specification) is absent” may, in an embodiment, be understood as R₁ (or R₂, R₃, R₄, X₅, R₆, or any other substituent or radical described in this specification) being H. In other words, when a substituent or radical is “absent”, it may in some embodiments be understood as being H.

The phrase “L′ is a bond to L” may, in an embodiment, be understood such that L′ does not represent a radical but a bond to L.

It may also be understood that not all atoms are drawn in the formulas described in this specification. Only substituents and groups that may vary have been drawn; H atoms may have been omitted for the sake of clarity.

The glycosylation inhibitor may, alternatively or additionally, be represented by formula II, wherein

X₁ is H, COOH, COOCH₃ or COOL′;

R₁ is absent, OH, OZ or L′;

R₂ is absent, Y, OH, OZ, NHCOCH₃ or L′;

R₃ is absent, Y, OH, OZ or L′;

R₄ is absent, Y, OH, OZ, NH₂, NR₄′R₄″, NHCOCH₃ or L′;

X₅ is absent, CH₂, CH(OH)CH₂, CH(OZ)CH₂, CH(OH)CH(OH)CH₂, CH(OZ)CH(OZ)CH₂, C₁-C₁₂ alkyl, or substituted C₁-C₁₂ alkyl;

R₆ is absent, Y, OH, OZ or L′;

L′ is a bond to L;

each Z is independently selected from COCH₃, C₁-C₁₂ acyl and substituted C₁-C₁₂ acyl;

Y is selected from F, Cl, Br, I, H and CH₃; and

R₄′ and R₄″ are each independently selected from H, C₁-C₁₂ alkyl, substituted C₁-C₁₂ alkyl, C₆-C₁₂ aryl, substituted C₆-C₁₂ aryl, COR₄″′ and COOR₄″′, wherein R₄″′ is selected from C₁-C₁₂ alkyl, substituted C₁-C₁₂ alkyl, C₆-C₁₂ aryl and substituted C₆-C₁₂ aryl;

with the proviso that not more than one of R₁, R₂, R₃, R₄ and R₆ are Y, that the glycosylation inhibitor contains not more than one L′, and when one of R₄′ and R₄″ is either COR₄′″ and COOR₄′″, then one of R₄′ and R₄″ is H.

In this context, the phrase “not more than one of R₁, R₂, R₃, R₄ and R₆ are Y” may be understood so that not more than one of R₁, R₂, R₃, R₄ and R₆ is selected from F, Cl, Br, I, H and CH₃.

The glycosylation inhibitor may, alternatively or additionally, be represented by formula II, wherein

X₁ is H, COOH, COOCH₃ or COOL′;

R₁ is absent, OH, OZ or L′;

R₂ is absent, Y, OH, OZ, NHCOCH₃ or L′;

R₃ is absent, Y, OH, OZ or L′;

R₄ is absent, Y, OH, OZ, NH₂, NR₄′R₄″, NHCOCH₃ or L′;

X₅ is absent, CH₂, CH(OH)CH₂, CH(OZ)CH₂, CH(OH)CH(OH)CH₂, CH(OZ)CH(OZ)CH₂, a C₁-C₁₂ alkyl, or a substituted C₁-C₁₂ alkyl;

R₆ is absent, Y, OH, OZ or L′;

L′ is a bond to L;

each Z is independently selected from COCH₃, a C₁-C₁₂ acyl and a substituted C₁-C₁₂ acyl; and

Y is selected from F, Cl, Br, I, H and CH₃; and

R₄′ and R₄″ are each independently selected from H, C₁-C₁₂ alkyl, substituted C₁-C₁₂ alkyl, C₆-C₁₂ aryl, substituted C₆-C₁₂ aryl, COR₄′″ and COOR₄′″, wherein R₄′″ is selected from C₁-C₁₂ alkyl, substituted C₁-C₁₂ alkyl, C₆-C₁₂ aryl and substituted C₆-C₁₂ aryl;

with the proviso that two of R₁, R₂, R₃, R₄ and R₆ are Y, that the glycosylation inhibitor contains not more than one L′, and when one of R₄′ and R₄″ is either COR₄′″ or COOR₄′″, then one of R₄′ and R₄″ is H.

In this context, the phrase “two of R₁, R₂, R₃, R₄ and R₆ are Y” may be understood so that two of R₁, R₂, R₃, R₄ and R₆ are selected from F, Cl, Br, I, H and CH₃.

The glycosylation inhibitor may, alternatively or additionally, be represented by formula II, wherein

X₁ is H, COOH, COOCH₃ or COOL′;

R₁ is absent, OH, OZ or L′;

R₂ is absent, Y, OH, OZ, NHCOCH₃ or L′;

R₃ is absent, Y, OH, OZ or L′;

R₄ is absent, Y, OH, OZ, NH₂, NR₄′R₄″, NHCOCH₃ or L′;

X₅ is absent, CH₂, CH(OH)CH₂, CH(OZ)CH₂, CH(OH)CH(OH)CH₂, CH(OZ)CH(OZ)CH₂, a C₁-C₁₂ alkyl, or a substituted C₁-C₁₂ alkyl;

R₆ is absent, Y, OH, OZ or L′;

L′ is a bond to L;

each Z is independently selected from COCH₃, a C₁-C₁₂ acyl and a substituted C₁-C₁₂ acyl;

Y is selected from F, Cl, Br, I, H and CH₃; and

R₄′ and R₄″ are each independently selected from H, C₁-C₁₂ alkyl, substituted C₁-C₁₂ alkyl, C₆-C₁₂ aryl, substituted C₆-C₁₂ aryl, COR₄′″ and COOR₄′″, wherein R₄′″ is selected from C₁-C₁₂ alkyl, substituted C₁-C₁₂ alkyl, C₆-C₁₂ aryl and substituted C₆-C₁₂ aryl;

with the proviso that three of R₁, R₂, R₃, R₄ and R₆ are Y, that the glycosylation inhibitor contains not more than one L′, and when one of R₄′ and R₄″ is either COR₄′″ and COOR₄′″, then one of R₄′ and R₄″ is H.

In this context, the phrase “three of R₁, R₂, R₃, R₄ and R₆ are Y” may be understood so that three of R₁, R₂, R₃, R₄ and R₆ are selected from F, Cl, Br, I, H and CH₃.

The term “substituted” in the context of Formula II may refer to being substituted by any one of the substituents described above.

Y may, in an embodiment of Formula II, be selected from F, Cl, Br, and I, or from F and Cl.

Y may, in an embodiment of Formula II, be F. Such fluorinated sugar analogs may be relatively effective glycosylation inhibitors, because the presence of the fluorine atom may prohibit the incorporation of the fluorinated sugar analog into various glycan structures. The fluorine atom also does not cause significant steric hindrance.

The glycosylation inhibitor may, alternatively or additionally, be represented by formula IIIa, IIIb, IIIc, IIId, IIIe, IIIf, IIIg or IIIh:

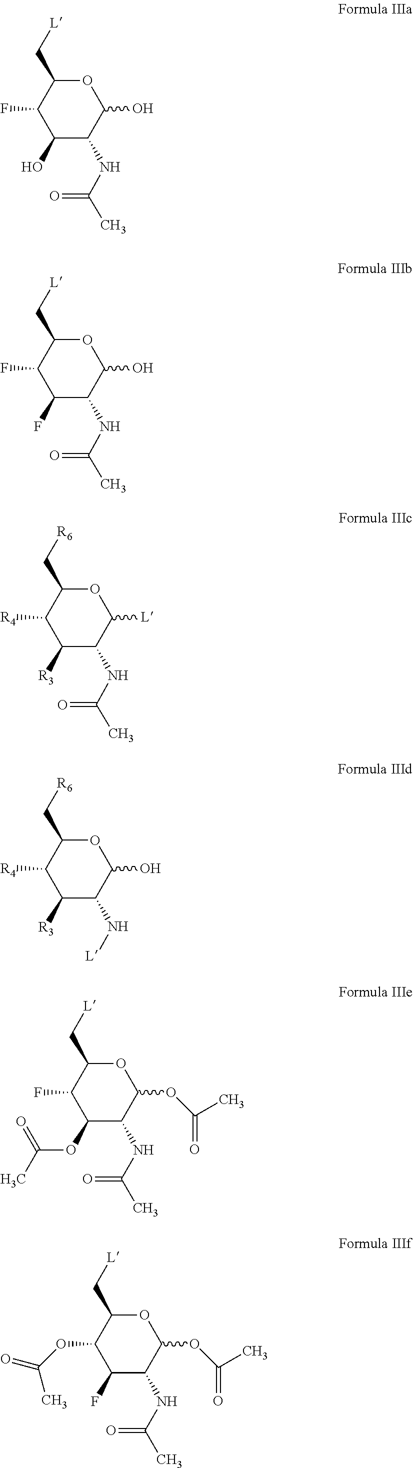

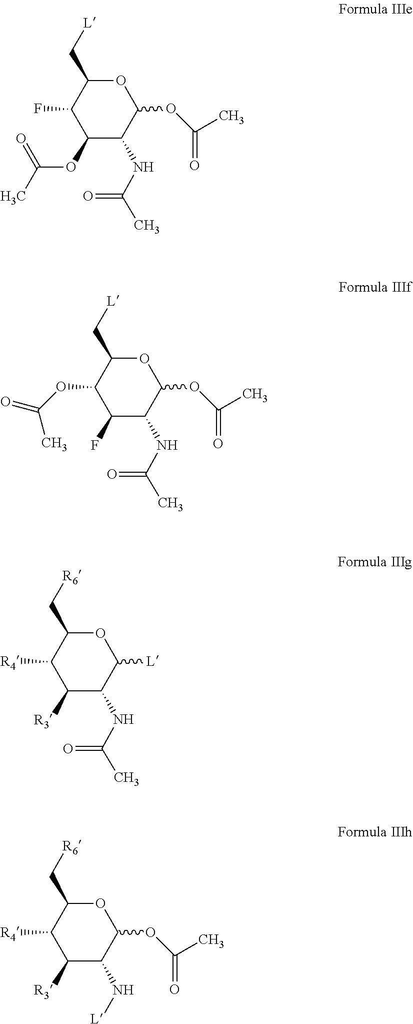

wherein L′ is a bond to L;

R₃, R₄ and R₆ are each independently either OH or F, with the proviso that only one of R₃, R₄ and R₆ is F; and

R₃′, R₄′ and R₆′ are each independently either OCOCH₃ or F, with the proviso that only one of R₃′, R₄′ and R₆′ is F.

The glycosylation inhibitor may, alternatively or additionally, be represented by any one of formulas IIIa, IIIb, IIIc, IIId, IIIe, IIIf, IIIg or IIIh, wherein L′ is a bond to L;

R₃, R₄ and R₆ are each independently either OH or F, with the proviso that two of R₃, R₄ and R₆ are F; and

R₃′, R₄′ and R₆′ are each independently either OCOCH₃ or F, with the proviso that two of R₃′, R₄′ and R₆′ are F.

The glycosylation inhibitor may, alternatively or additionally, be represented by any one of formulas IIIa, IIIb, IIIc, IIId, IIIe, IIIf, IIIg or IIIh, wherein L′ is a bond to L;

R₃, R₄ and R₆ are each F; and

R₃′, R₄′ and R₆′ are each F.

In an embodiment, the glycosylation inhibitor is a 3-deoxy-3-fluorosialic acid. In an embodiment, the 3-deoxy-3-fluoro-sialic acid is a 3-deoxy-3ax-fluorosialic acid or a 3-deoxy-3eq-fluorosialic acid.

The 3-deoxy-3-fluorosialic acid may, alternatively or additionally, be represented by any one of formulas IVa, IVb, IVc, IVd, IVe or IVf:

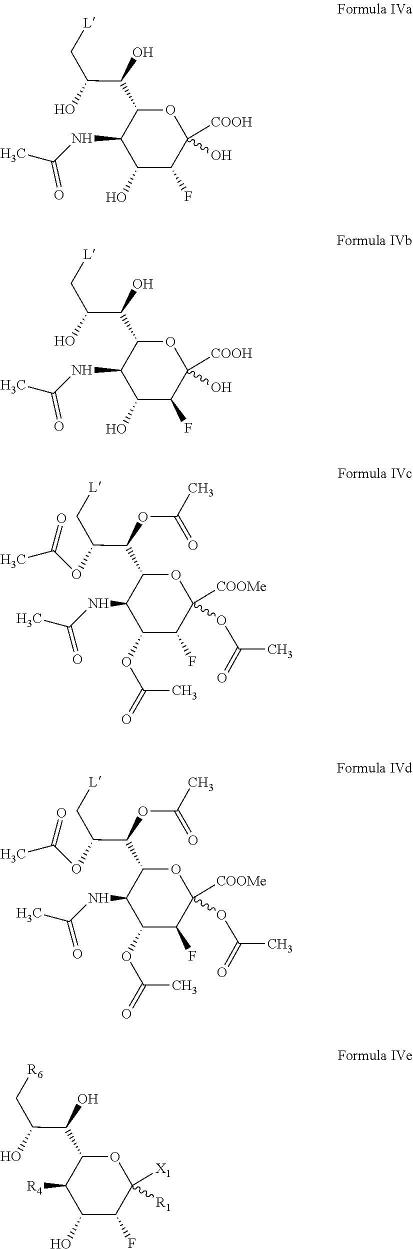

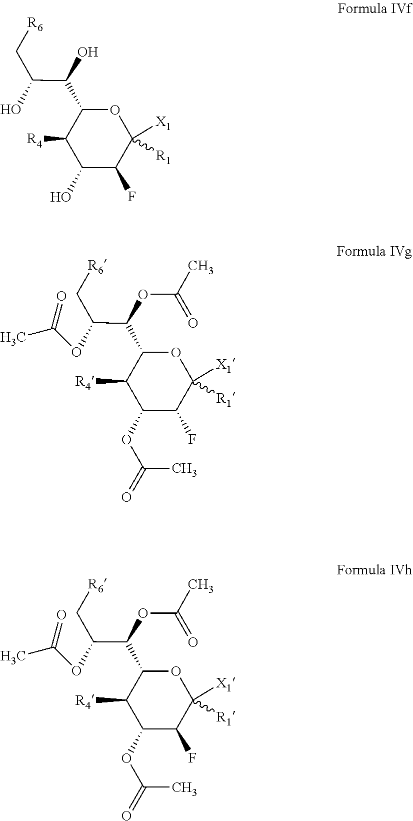

wherein

L′ is a bond to L;

R₁ and R₆ are each independently either OH or L′, R₄ is independently either NHCOCH₃ or L′, and X₁ is independently either COOH or L′, with the proviso that only one of R₁, R₄, R₆ and X₁ is L′; and

R₁′ and R₆′ are each independently either OCOCH₃ or L′;

R₄′ is independently either NHCOCH₃ or L′, and

X₁′ is independently either COOCH₃ or L′,

with the proviso that only one of R₁′, R₄′, R₆′ and X₁′ is L′.

In the context of this specification, the phrase “3-deoxy-3-fluorosialic acid” may be understood so that one of the hydrogen atoms bonded to carbon-3 of the sialic acid is replaced by a fluorine atom. In this context, the phrase “3-deoxy-3ax-fluoro-sialic acid” may be understood so that the axial hydrogen atom bonded to carbon-3 of the sialic acid is replaced by a fluorine atom. In this context, the phrase “3-deoxy-3eq-fluorosialic acid” may be understood so that the equatorial hydrogen atom bonded to carbon-3 of the sialic acid is replaced by a fluorine atom.

The 3-deoxy-3-fluorosialic acid may, alternatively or additionally, be represented by any one of formulas IVe, IVf, IVg or IVh, wherein:

L′ is a bond to L;

R₁ and R₆ are each independently either OH, OZ or L′;

R₄ and R₄′ are independently either absent, OH, OZ, NH₂, NR₄′″R₄′″, NHL′, NHCOCH₃ or L′;

X₁ is independently either COOH, COOMe, COOL′ or L′; each Z is independently selected from COCH₃, a C₁-C₁₂ acyl and a substituted C₁-C₁₂ acyl;

R₁′ and R₆′ are each independently either OH, OZ, OCOCH₃ or L′;

R₄″ and R₄″ are each independently selected from H, C₁-C₁₂ alkyl, substituted C₁-C₁₂ alkyl, C₆-C₁₂ aryl, substituted C₆-C₁₂ aryl, COR₄″″ and COOR₄″″, L′, L″-L′, Y, NH₂, OH, NHCOCH₃, NHCOCH₂OH, NHCOCF₃, NHCOCH₂Cl, NHCOCH₂OCOCH₃, NHCOCH₂N₃, NHCOCH₂CH₂CCH, NHCOOCH₂CCH, NHCOOCH₂CHCH₂, NHCOOCH₃, NHCOOCH₂CH₃, NHCOOCH₂CH(CH₃)₂, NHCOOC(CH₃)₃, NHCOO-benzyl, NHCOOCH₂-1-benzyl-1H-1,2,3-triazol-4-yl, NHCOO(CH₂)₃CH₃, NHCOO(CH₂)₂OCH₃, NHCOOCH₂CCl₃ and NHCOO(CH₂)₂F (wherein benzyl=CH₂C₆H₅);

wherein R₄″″ is selected from C₁-C₁₂ alkyl, substituted C₁-C₁₂ alkyl, C₆-C₁₂ aryl and substituted C₆-C₁₂ aryl;

L″ is selected from L′-substituted C₁-C₁₂ alkyl, L′-substituted C₆-C₁₂ aryl, COL′″, COOL′″, NH—, O—, NHCOCH₂—, NHCOCH₂O, NHCOCF₂—, NHCOCH₂OCOCH₂—, NHCOCH₂triazolyl-, NHCOOCH₂CHCH—, NHCOOCH₂CH₂CH₂S—, NHCOOCH₂—, NHCOOCH₂CH₂—, NHCOOCH₂CHCH₂CH₂—, NHCOO-benzyl-, NHCOO(CH₂)₃CH₂—, NHCOOCH₂—1-benzyl-1H-1,2,3-triazol-4-yl and NHCOO(CH₂)₂OCH₂— (wherein benzyl is CH₂C₆H₅ and - is the bond to L′);

wherein L′″ is either L′-substituted C₁-C₁₂ alkyl or L′-substituted C₆-C₁₂ aryl,

with the proviso that the glycosylation inhibitor contains not more than one L′, and when R₄′ is either COR₄′″ or COOR₄′″ then R₄″ is H, and when R₄″ is either COR₄′″ or COOR₄′″ then R₄′ is H.

In the context of this specification, the term “L′-substituted” may be understood as referring to comprising L′, i.e. a bond to L. In other words, L′″ may be bonded to L.

The 3-deoxy-3-fluorosialic acid may, alternatively or additionally, be represented by any one of formulas IVi, IVj, IVk, IVl or IVm:

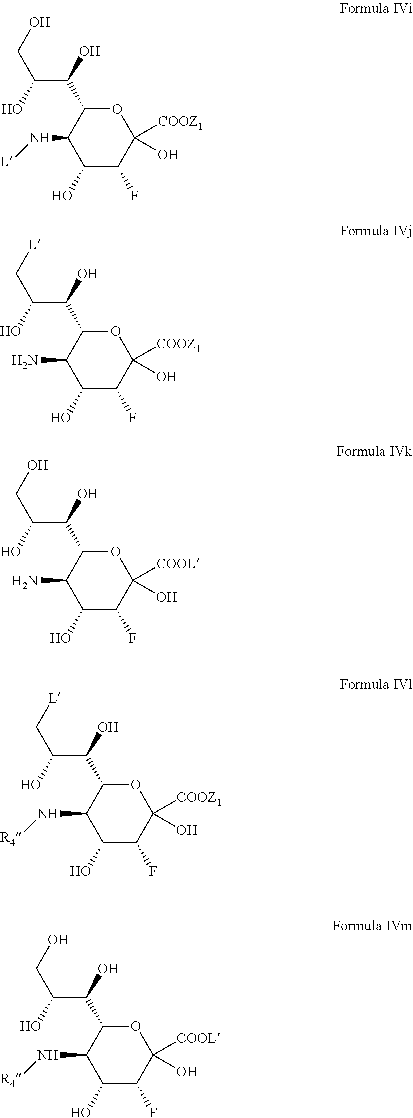

wherein

L′ is a bond to L;

Z₁ is selected from H, CH₃, C₁-C₁₂ alkyl, substituted C₁-C₁₂ alkyl, C₆-C₁₂ aryl and substituted C₆-C₁₂ aryl; and R₄″ is selected from C₁-C₁₂ alkyl, substituted C₁-C₁₂ alkyl,

C₆-C₁₂ aryl, substituted C₆-C₁₂ aryl, COR₄″″, COOR₄″″, COCH₃, COCH₂OH, COCF₃, COCH₂Cl, COCH₂OCOCH₃, COCH₂N₃, COCH₂CH₂CCH, COOCH₂CCH, COOCH₂CHCH₂, COOCH₃, COOCH₂CH₃, COOCH₂CH(CH₃)₂, COOC(CH₃)₃, COO-benzyl, COOCH₂-1-benzyl-1H-1,2,3-triazol-4-yl, COO(CH₂)₃CH₃, COO(CH₂)₂OCH₃, COOCH₂CCl₃ and COO(CH₂)₂F (wherein benzyl=CH₂C₆H₅);

wherein R₄″″ is selected from C₁-C₁₂ alkyl, substituted C₁-C₁₂ alkyl, C₆-C₁₂ aryl and substituted C₆-C₁₂ aryl.

The glycosylation inhibitor may, alternatively or additionally, be represented by formula A:

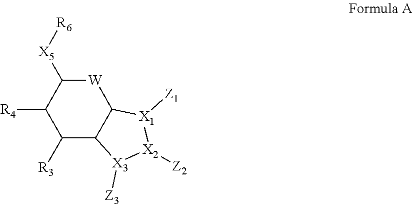

wherein

W is CH₂, NH, O or S;

X₁, X₂ and X₃ are each independently selected from S, O, C, CH and N;

with the proviso that when one or both of X₁ and X₃ are either O or S, then X₂ is either absent, a bond between X₁ and X₂, or CH;

Z₁, Z₂ and Z₃ are each independently either absent or selected from H, OH, OZ, ═O, (═O)₂, C₁-C₁₂ alkyl, substituted C₁-C₁₂ alkyl, C₆-C₁₂ aryl, substituted C₆-C₁₂ aryl or L′;

R₃ and R₄ are are each independently either absent or selected from H, OH, OZ or L′;

X₅ is absent, OH, OZ, O, CH₂, C₁-C₁₂ alkyl, or substituted C₁-C₁₂ alkyl;

R₆ is absent, H, OH, OZ, a phosphate, a phosphate ester, a phosphate analog, a boronophosphate, a boronophosphate ester, a thiophosphate, a thiophosphate ester, a halophosphate, a halophosphate ester, a vanadate, a phosphonate, a phosphonate ester, a thiophosphonate, a thiophosphonate ester, a halophosphonate, a halophosphonate ester, methylphosphonate, methylphosphonate ester or L′;

L′ is a bond to L;

each Z is independently selected from COCH₃, C₁-C₁₂ acyl and substituted C₁-C₁₂ acyl; and

each of the bonds between the ring carbon and X₃, X₂ and X₃, X₁ and X₂, and the ring carbon and X₁, are independently either a single bond or a double bond or absent;

with the proviso than when both of the bonds between X₂ and X₃, and X₁ and X₂, are absent, then both X₂ and Z₂ are also absent;

with the proviso that the glycosylation inhibitor contains not more than one L′.

The glycosylation inhibitor may, alternatively or additionally, be represented by formula Aa, Ab, Ac or Ad:

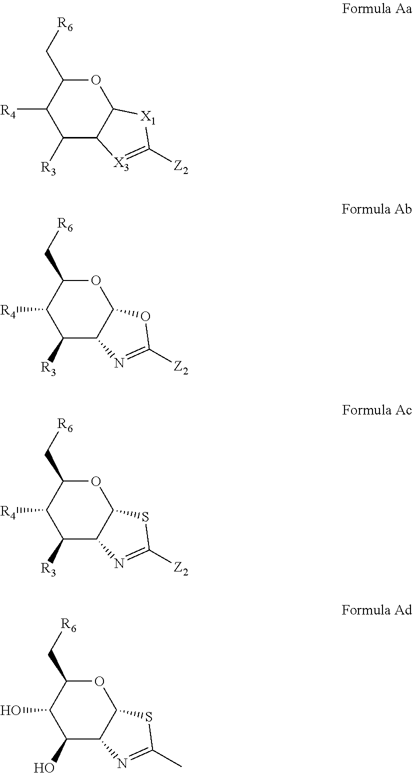

wherein

X₁ is selected from S, O, CH₂ and NH;

X₃ is selected from CH and N;

Z₂ is either absent or selected from H, OH, OZ, ═O, (═O)₂, C₁-C₁₂ alkyl, substituted C₁-C₁₂ alkyl, C₆-C₁₂ aryl, substituted C₆-C₁₂ aryl or L′;

R₃ and R₄ are are each independently either absent or selected from H, OH, OZ or L′;

R₆ is absent, H, OH, OZ, a phosphate, a phosphate ester, a phosphate analog, a thiophosphate, a thiophosphate ester, a halophosphate, a halophosphate ester, a vanadate, a phosphonate, a phosphonate ester, a thiophosphonate, a thiophosphonate ester, a halophosphonate, a halophosphonate ester, methylphosphonate, methylphosphonate ester or L′;

L′ is a bond to L; and

each Z is independently selected from COCH₃, C₁-C₁₂ acyl and substituted C₁-C₁₂ acyl;

with the proviso that the glycosylation inhibitor contains not more than one L′.

The glycosylation inhibitor may, alternatively or additionally, be represented by formula B:

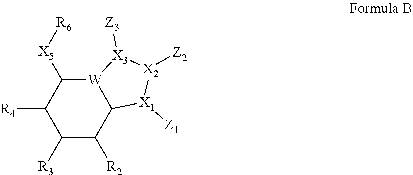

wherein

W is CH, N, O or S;

X₁, X₂ and X₃ are each independently selected from S, O, CH and N;

with the proviso that when one or both of X₁ and X₃ are either O or S, then X₂ is either absent, a bond between X₁ and X₃, C or CH;

Z₁, Z₂ and Z₃ are each independently either absent or selected from H, OH, OZ, ═O, (═O)₂, C₁-C₁₂ alkyl, substituted C₁-C₁₂ alkyl, C₆-C₁₂ aryl, substituted C₆-C₁₂ aryl or L′;

R₂, R₃ and R₄ are are each independently either absent or selected from H, OH, OZ or L′;

X₅ is absent, OH, OZ, O, CH₂, C₁-C₁₂ alkyl, or substituted C₁-C₁₂ alkyl;

R₆ is absent, H, OH, OZ or L′;

L′ is a bond to L;

each Z is independently selected from COCH₃, C₁-C₁₂ acyl and substituted C₁-C₁₂ acyl; and

each of the bonds between W and X₃, X₂ and X₃, X₁ and X₂, and the ring carbon and X₁, are independently either a single bond or a double bond or absent;

with the proviso than when both of the bonds between X₂ and X₃, and X₁ and X₂, are absent, then both X₂ and Z₂ are also absent;

with the proviso that the glycosylation inhibitor contains not more than one L′.

The glycosylation inhibitor may, alternatively or additionally, be represented by formula Ba, Bb, Bc, Bd, Be, Bf, Bg or Bh:

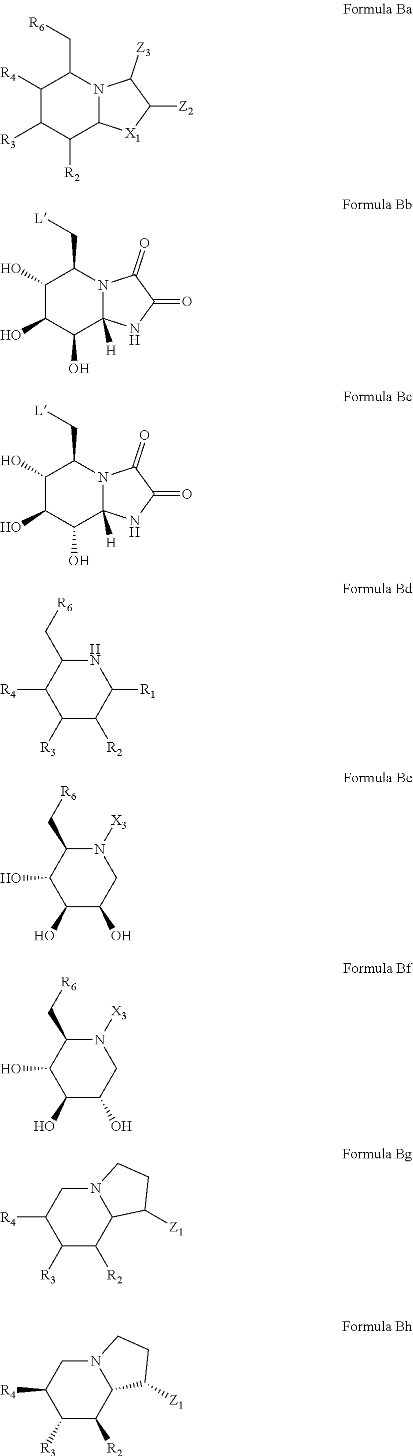

wherein

X₁ is selected from S, O, CH₂ and NH;

X₃ is selected from H, C₁-C₁₂ alkyl, substituted C₁-C₁₂ alkyl, C₁-C₁₂ acyl, substituted C₁-C₁₂ acyl, C₆-C₁₂ aryl, substituted C₆-C₁₂ aryl or L′

Z₁, Z₂ and Z₃ are each independently either absent or selected from H, OH, OZ, ═O, (═O)₂, C₁-C₁₂ alkyl, substituted C₁-C₁₂ alkyl, C₆-C₁₂ aryl, substituted C₆-C₁₂ aryl or L′;

R₁, R₂, R₃ and R₄ are are each independently either absent or selected from H, OH, OZ or L′;

R₆ is absent, H, OH, OZ or L′;

L′ is a bond to L; and

each Z is independently selected from COCH₃, C₁-C₁₂ acyl and substituted C₁-C₁₂ acyl;

with the proviso that the glycosylation inhibitor contains not more than one L′.

The glycosylation inhibitor may, alternatively or additionally, be represented by formula Ca, Cb or Cc:

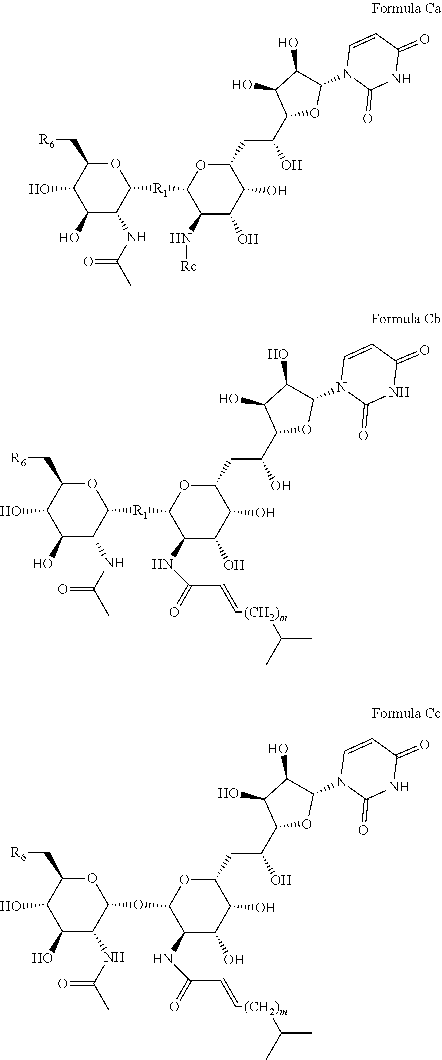

wherein

R₁ is O, NH, NRb, S, SO, SO₂ or CH₂;

Rb is C₁-C₁₀ alkyl, substituted C₁-C₁₀ alkyl, C₁-C₁₀ acyl or substituted C₁-C₁₀ acyl;

R₆ is OH or L′;

Rc is C₂-C₂₀ acyl, substituted C₂-C₂₀ acyl, C₆-C₂₀ aryl, substituted C₆-C₂₀ aryl or L′;

m is 6, 7, 8, 9, 10, 11, 12, 13 or 14; and

L′ is a bond to L.

The glycosylation inhibitor may, alternatively or additionally, be represented by formula Da, Db or Dc:

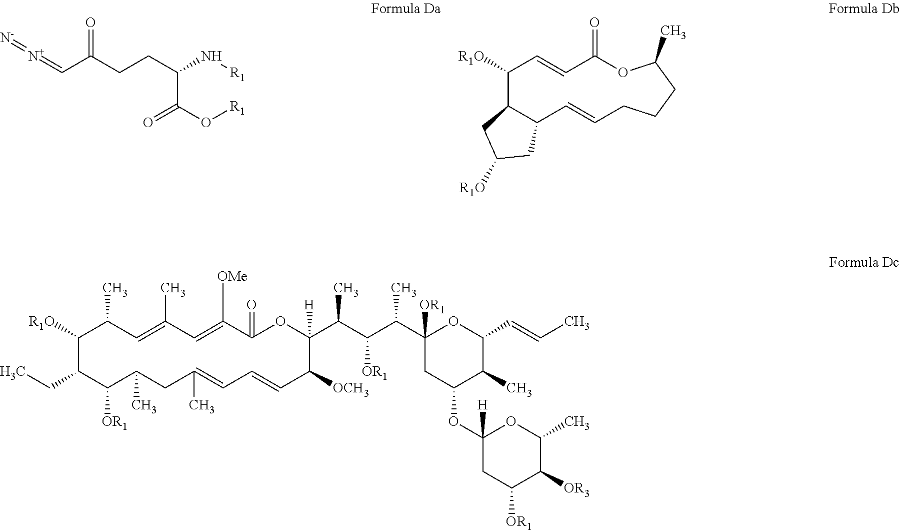

wherein

each R₁ is independently either H or L′;

R₃ is H, OH, CONH₂, CONHL′ or L′; and

L′ is a bond to L;

with the proviso that each of the Formulas Da, Db and Dc contain only one L′.

The glycosylation inhibitor according to one or more embodiments described in this specification may be conjugated to the targeting unit in various ways.

III) Linker Units

Various types of linker units may be suitable, and many are known in the art. The linker unit may comprise one or more linker groups or moieties. It may also comprise one or more groups formed by a reaction between two functional groups. A skilled person will realize that various different chemistries may be utilized when preparing the conjugate, and thus a variety of different functional groups may be reacted to form groups comprised by the linker unit L. In an embodiment, the functional groups are selected from the group consisting of sulfhydryl, amino, alkenyl, alkynyl, azidyl, aldehyde, carboxyl, maleimidyl, succinimidyl and hydroxylamino. A skilled person is capable of selecting the functional groups so that they may react in certain conditions.

The terms “linker unit” and “linker” may be used interchangeably in this specification.

The linker unit may be configured to release the glycosylation inhibitor after the conjugate is bound to the target cell. The linker unit may, for example, be cleavable. The cleavable linker unit may be cleavable under intracellular conditions, such that the cleavage of the linker unit may release the glycosylation inhibitor in the intracellular environment. The cleavable linker unit may be cleavable under conditions of the tumour microenvironment, such that the cleavage of the linker unit may release the glycosylation inhibitor in the tumour.

The linker unit may be configured to release the glycosylation inhibitor after the conjugate is delivered to the tumour and/or bound to the target molecule or to the target cell.

The linker unit may be non-cleavable.

The linker unit may be cleavable by a cleaving agent that is present in the intracellular environment (e.g., within a lysosome or endosome) or in the tumour microenvironment. The linker unit can be e.g. a peptidyl linker unit that is cleaved by an intracellular peptidase or protease enzyme, for example a lysosomal or endosomal protease, or a peptidase or a protease of the tumour microenvironment. In some embodiments, the peptidyl linker unit is at least two amino acids long or at least three amino acids long. Cleaving agents can include e.g. cathepsins B and D, plasmin, and a matrix metalloproteinase. The peptidyl linker unit cleavable by an intracellular protease or a tumour microenvironment protease may be a Val-Cit linker or a Phe-Lys linker.

The linker unit may be cleavable by a lysosomal hydrolase or a hydrolase of the tumour microenvironment. In an embodiment, the linker unit can comprise a glycosidic bond that is cleavable by an intracellular glycosidase enzyme, for example a lysosomal or endosomal glycosidase, or a glycosidase of the tumour microenvironment. In some embodiments, the glycosidic linker unit comprises a monosaccharide residue or a larger saccharide. Cleaving agents can include e.g. β-glucuronidase, β-galactosidase and β-glucosidase. The glycosidic linker unit cleavable by an intracellular glycosidase or a tumour microenvironment glycosidase may be a β-D-glucuronide linker unit, a β-galactoside linker unit or a β-glucoside linker unit.

The cleavable linker unit may be pH-sensitive, i.e. sensitive to hydrolysis at certain pH values, for example under acidic conditions. For example, an acid-labile linker unit that is hydrolyzable in the lysosome or the tumour microenvironment {e.g., a hydrazone, semicarbazone, thiosemicarbazone, cis-aconitic amide, orthoester, acetal, ketal, or the like) can be used. Such linker units are relatively stable under neutral pH conditions, such as those in the blood, but are unstable at below pH 5.5 or 5.0, or at at below pH 4.5 or 4.0, the approximate pH of the lysosome. In an embodiment, the hydrolyzable linker unit is a thioether linker unit.

The linker unit may be cleavable under reducing conditions, e.g. a disulfide linker unit, examples of which may include disulfide linker units that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonylalpha-methyl-alpha-(2-pyridyl-dithio)toluene), SPDB and SMPT.

The linker unit may be a malonate linker, a maleimidobenzoyl linker, or a 3′-N-amide analog.

L, i.e. the linker unit, in Formula I may, in an embodiment, be represented by formula IX

—R₇-L₁-Sₚ-L₂-R₈— Formula IX

wherein

R₇ is a group covalently bonded to the glycosylation inhibitor;

L₁ is a spacer unit or absent;

Sₚ is a specificity unit or absent;

L₂ is a stretcher unit covalently bonded to the targeting unit or absent; and

R₈ is absent or a group covalently bonded to the targeting unit.

R₇ may, for example, be selected from:

—C(═O)NH—,

—C(═O)O—,

—NHC(═O)—,

—OC(═O)—, —OC(═O)O—,

—NHC(═O)O—,

—OC(═O)NH—,

—NHC(═O)NH,

—NH—,

—S— and

—O—.

The group —O— may in this context be understood as an oxygen atom forming a glycosidic bond between the glycosylation inhibitor and L₈, Sₚ, L₂, R₈ or T (whichever present).

R₈ may, for example, be selected from:

—C(═O)NH—,

—C(═O)O—,

—NHC(═O)—,

—OC(═O)—,

—OC(═O)O—,

—NHC(═O)O—,

—OC(═O)NH—,

—NHC(═O)NH,

—NH—,

—S— and

—O—.

The group —O— may also in the context of R₈ be understood as an oxygen atom forming a glycosidic bond between the targeting unit and L₁, L₂ or Sₚ.

IV) Targeting Units

In an embodiment, the targeting unit is a targeting unit that is capable of binding an immune checkpoint molecule. In an embodiment, the immune checkpoint molecule is any molecule involved in immune checkpoint function. In an embodiment, the immune checkpoint molecule is a checkpoint protein as defined by the NCI Dictionary of Cancer Terms available at https://www.cancer.gov/publications/dictionaries/cancerterms/dPf/immune-checkpoint-inhibitor. In an embodiment, the immune checkpoint molecule is a target molecule of an immune checkpoint inhibitor as defined by the NCI Dictionary of Cancer Terms available at https://www.cancer.gov/publications/dictionaries/cancerterms/def/immune-checkpoint-inhibitor. In an embodiment, the immune checkpoint molecule is any molecule described in Marin-Acevedo et al. 2018, J Hematol Oncol 11:39.

In an embodiment, the immune checkpoint molecule is selected from the group of PD-1, PD-L1, CTLA-4, lymphocyte activation gene-3 (LAG-3, CD223), T cell immunoglobulin-3 (TIM-3), poly-N-acetyllactosamine, T (Thomsen-Friedenreich) antigen, Globo H, Lewis c (type 1 N-acetyllactosamine), Galectin-1, Galectin-2, Galectin-3, Galectin-4, Galectin-5, Galectin-6, Galectin-7, Galectin-8, Galectin-9, Galectin-10, Galectin-11, Galectin-12, Galectin-13, Galectin-14, Galectin-15, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, Siglec-17, phosphatidyl serine, CEACAM-1, T cell immunoglobulin and ITIM domain (TIGIT), CD155 (poliovirus receptor-PVR), CD112 (PVRL2, nectin-2), V-domain Ig suppressor of T cell activation (VISTA, also known as programmed death-1 homolog, PD-1H), B7 homolog 3 (B7-H3, CD276), adenosine A2a receptor (A2aR), CD73, B and T cell lymphocyte attenuator (BTLA, CD272), herpes virus entry mediator (HVEM), transforming growth factor (TGF)-13, killer immunoglobulin-like receptor (KIR, CD158), KIR2DL1/2L3, KIR3DL2, phosphoinositide 3-kinase gamma (PI3Kγ), CD47, OX40 (CD134), Glucocorticoid-induced TNF receptor family-related protein (GITR), GITRL, Inducible co-stimulator (ICOS), 4-1BB (CD137), CD27, CD70, CD40, CD154, indoleamine-2,3-dioxygenase (IDO), toll-like receptors (TLRs), TLR1, TLR2, TLR3, TLR4, TLR5, TLR6, TLR7, TLR8, TLR9, interleukin 12 (IL-12), IL-2, IL-2R, CD122 (IL-2Rβ), CD132 (γ_{c}), CD25 (IL-2Rα), and an arginase.

The targeting unit may comprise or be an antibody. For example, the targeting unit may be a tumour cell-targeting antibody, a cancer-targeting antibody and/or an immune cell-targeting antibody. The conjugate may therefore be an antibody-glycosylation inhibitor conjugate.

In an embodiment, the targeting unit is a bispecific targeting molecule capable of binding to two different target molecules at the same time. In an embodiment, the bispecific targeting unit is a bispecific antibody.

The targeting unit may, alternatively or additionally, comprise or be a peptide, an aptamer, or a glycan.

The targeting unit may, alternatively or additionally, comprise or be a cancer-targeting molecule, such as a ligand of a cancer-associated receptor. Examples of such cancer-targeting molecules include but are not limited to folate.

The targeting unit may further comprise one or more modifications, such as one or more glycosylations or glycans. For example, antibodies typically have one or more glycans. These glycans may be naturally occurring or modified. The glycosylation inhibitor may, in some embodiments, be conjugated to a glycan of the targeting unit, such as an antibody. In some embodiments, the targeting unit may comprise one or more further groups or moieties, for example a functional group or moiety (e.g. a fluorescent or otherwise detectable label).

The targeting unit may comprise or be, for example, a cancer-targeting antibody selected from the group of bevacizumab, tositumomab, etanercept, trastuzumab, adalimumab, alemtuzumab, gemtuzumab ozogamicin, efalizumab, rituximab, infliximab, abciximab, basiliximab, palivizumab, omalizumab, daclizumab, cetuximab, panitumumab, epratuzumab, 2G12, lintuzumab, nimotuzumab and ibritumomab tiuxetan.

The targeting unit may, in an embodiment, comprise or be an antibody selected from the group of an anti-EGFR1 antibody, an epidermal growth factor receptor 2 (HER2/neu) antibody, an anti-CD22 antibody, an anti-CD30 antibody, an anti-CD33 antibody, an anti-Lewis y antibody, an anti-CD20 antibody, an anti-CD3 antibody, an anti-PSMA antibody, an anti-TROP2 antibody and an anti-AXL antibody.

The target molecule may, in an embodiment, comprise or be a molecule selected from the group of EGFR1, epidermal growth factor receptor 2 (HER2/neu), CD22, CD30, CD33, Lewis y, CD20, CD3, PSMA, trophoblast cell-surface antigen 2 (TROP2) and tyrosine-protein kinase receptor UFO (AXL).

The targeting unit may, in an embodiment, comprise or be an immune checkpoint molecule-targeting antibody selected from the group of nivolumab, pembrolizumab, ipilimumab, atezolizumab, avelumab, durvalumab, BMS-986016, LAG525, MBG453, OMP-31M32, JNJ-61610588, enoblituzumab (MGA271), MGD009, 8H9, MEDI9447, M7824, metelimumab, fresolimumab, IMC-TR1 (LY3022859), lerdelimumab (CAT-152), LY2382770, lirilumab, IPH4102, 9B12, MOXR 0916, PF-04518600 (PF-8600), MEDI6383, MEDI0562, MEDI6469, INCAGN01949, GSK3174998, TRX-518, BMS-986156, AMG 228, MEDI1873, MK-4166, INCAGN01876, GWN323, JTX-2011, GSK3359609, MEDI-570, utomilumab (PF-05082566), urelumab, ARGX-110, BMS-936561 (MDX-1203), varlilumab, CP-870893, APX005M, ADC-1013, lucatumumab, Chi Lob 7/4, dacetuzumab, SEA-CD40, 807009789, and MEDI9197.

The targeting unit may comprise or be a molecule selected from the group of an immune checkpoint inhibitor, an anti-immune checkpoint molecule, anti-PD-1, anti-PD-L1 antibody, anti-CTLA-4 antibody, or an antibody targeting an immune checkpoint molecule selected from the group of: lymphocyte activation gene-3 (LAG-3, CD223), T cell immunoglobulin-3 (TIM-3), poly-N-acetyllactosamine, T (Thomsen-Friedenreich antigen), Globo H, Lewis c (type 1 N-acetyllactosamine), Galectin-1, Galectin-2, Galectin-3, Galectin-4, Galectin-5, Galectin-6, Galectin-7, Galectin-8, Galectin-9, Galectin-10, Galectin-11, Galectin-12, Galectin-13, Galectin-14, Galectin-15, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, Siglec-17, phosphatidyl serine, CEACAM-1, T cell immunoglobulin and ITIM domain (TIGIT), CD155 (poliovirus receptor-PVR), CD112 (PVRL2, nectin-2), V-domain Ig suppressor of T cell activation (VISTA, also known as programmed death-1 homolog, PD-1H), B7 homolog 3 (B7-H3, CD276), adenosine A2a receptor (A2aR), CD73, B and T cell lymphocyte attenuator (BTLA, CD272), herpes virus entry mediator (HVEM), transforming growth factor (TGF)-β, killer immunoglobulin-like receptor (KIR, CD158), KIR2DL1/2L3, KIR3DL2, phosphoinositide 3-kinase gamma (PI3Kγ), CD47, OX40 (CD134), Glucocorticoid-induced TNF receptor family-related protein (GITR), GITRL, Inducible co-stimulator (ICOS), 4-1BB (CD137), CD27, CD70, CD40, CD154, indoleamine-2,3-dioxygenase (IDO), toll-like receptors (TLRs), TLR1, TLR2, TLR3, TLR4, TLR5, TLR6, TLR7, TLR8, TLR9, interleukin 12 (IL-12), IL-2, IL-2R, CD122 (IL-2Rβ), CD132 (γ_{c}), CD25 (IL-2Rα), and arginase.

The target molecule may comprise or be a molecule selected from the group of an immune checkpoint molecule, PD-1, PD-L1, CTLA-4, lymphocyte activation gene-3 (LAG-3, CD223), T cell immunoglobulin-3 (TIM-3), poly-N-acetyllactosamine, T (Thomsen-Friedenreich antigen), Globo H, Lewis c (type 1 N-acetyllactosamine), Galectin-1, Galectin-2, Galectin-3, Galectin-4, Galectin-5, Galectin-6, Galectin-7, Galectin-8, Galectin-9, Galectin-10, Galectin-11, Galectin-12, Galectin-13, Galectin-14, Galectin-15, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, Siglec-17, phosphatidyl serine, CEACAM-1, T cell immunoglobulin and ITIM domain (TIGIT), CD155 (poliovirus receptor-PVR), CD112 (PVRL2, nectin-2), V-domain Ig suppressor of T cell activation (VISTA, also known as programmed death-1 homolog, PD-1H), B7 homolog 3 (B7-H3, CD276), adenosine A2a receptor (A2aR), CD73, B and T cell lymphocyte attenuator (BTLA, CD272), herpes virus entry mediator (HVEM), transforming growth factor (TGF)-β, killer immunoglobulin-like receptor (KIR, CD158), KIR2DL1/2L3, KIR3DL2, phosphoinositide 3-kinase gamma (PI3Kγ), CD47, OX40 (CD134), Glucocorticoid-induced TNF receptor family-related protein (GITR), GITRL, Inducible co-stimulator (ICOS), 4-1BB (CD137), CD27, CD70, CD40, CD154, indoleamine-2,3-dioxygenase (IDO), toll-like receptors (TLRs), TLR1, TLR2, TLR3, TLR4, TLR5, TLR6, TLR7, TLR8, TLR9, interleukin 12 (IL-12), IL-2, IL-2R, CD122 (IL-2Rβ), CD132 (γ_{c}), CD25 (IL-2Rα), and arginase.

V) Stretcher Units

The term “stretcher unit” may refer to any group, moiety or linker portion capable of linking R₇, L₁, or Sₚ (whichever present) to R₈ (if present) or to the targeting unit. Various types of stretcher units may be suitable, and many are known in the art.

The stretcher unit L₂ may have a functional group that can form a bond with a functional group of the targeting unit. The stretcher unit may also have a functional group that can form a bond with a functional group of either R₇, L₁, or Sₚ. Useful functional groups that can be present on the targeting unit, either naturally or via chemical manipulation, include, but are not limited to, sulfhydryl (—SH), amino, hydroxyl, carboxy, the anomeric hydroxyl group of a carbohydrate, and carboxyl. The functional groups of the targeting unit may, in an embodiment, be sulfhydryl and amino. The stretcher unit can comprise for example, a maleimide group, an aldehyde, a ketone, a carbonyl, or a haloacetamide for attachment to the targeting unit.

In one example, sulfhydryl groups can be generated by reduction of the intramolecular disulfide bonds of a targeting unit, such as an antibody. In another embodiment, sulfhydryl groups can be generated by reaction of an amino group of a lysine moiety of an antibody or other targeting unit with 2-iminothiolane (Traut's reagent) or other sulfhydryl generating reagents. In certain embodiments, the targeting unit is a recombinant antibody and is engineered to carry one or more lysines. In certain other embodiments, the recombinant antibody is engineered to carry additional sulfhydryl groups, e.g. additional cysteines.

In an embodiment, the stretcher unit has an electrophilic group that is reactive to a nucleophilic group present on the targeting unit (e.g. an antibody). Useful nucleophilic groups on the targeting unit include but are not limited to, sulfhydryl, hydroxyl and amino groups. The heteroatom of the nucleophilic group of the targeting unit is reactive to an electrophilic group on a stretcher unit and forms a covalent bond to the stretcher unit. Useful electrophilic groups include, but are not limited to, maleimide and haloacetamide groups. For an antibody as the targeting unit, the electrophilic group may provide a convenient site for antibody attachment for those antibodies having an accessible nucleophilic group.

In another embodiment, the stretcher unit has a reactive site which has a nucleophilic group that is reactive to an electrophilic group present on a targeting unit (e.g. an antibody). Useful electrophilic groups on a targeting unit include, but are not limited to, aldehyde and ketone and carbonyl groups. The heteroatom of a nucleophilic group of the stretcher unit can react with an electrophilic group on the targeting unit and form a covalent bond to the targeting unit, e.g. an antibody. Useful nucleophilic groups on the stretcher unit include, but are not limited to, hydrazide, hydroxylamine, amino, hydrazine, thiosemicarbazone, hydrazine carboxylate, and arylhydrazide. For an antibody as the targeting unit, the electrophilic group on the antibody may provide a convenient site for attachment to a nucleophilic stretcher unit.

In an embodiment, the stretcher unit has a reactive site which has an electrophilic group that is reactive with a nucleophilic group present on a targeting unit, such as an antibody. The electrophilic group provides a convenient site for the targeting unit (e.g., antibody) attachment. Useful nucleophilic groups on an antibody include but are not limited to, sulfhydryl, hydroxyl and amino groups. The heteroatom of the nucleophilic group of an antibody is reactive to an electrophilic group on the stretcher unit and forms a covalent bond to the stretcher unit. Useful electrophilic groups include, but are not limited to, maleimide and haloacetamide groups and NHS esters.

In another embodiment, a stretcher unit has a reactive site which has a nucleophilic group that is reactive with an electrophilic group present on the targeting unit. The electrophilic group on the targeting unit (e.g. antibody) provides a convenient site for attachment to the stretcher unit. Useful electrophilic groups on an antibody include, but are not limited to, aldehyde and ketone carbonyl groups. The heteroatom of a nucleophilic group of the stretcher unit can react with an electrophilic group on an antibody and form a covalent bond to the antibody. Useful nucleophilic groups on the stretcher unit include, but are not limited to, hydrazide, oxime, amino, hydrazine, thiosemicarbazone, hydrazine carboxylate, and arylhydrazide.

In some embodiments, the stretcher unit forms a bond with a sulfur atom of the targeting unit via a maleimide group of the stretcher unit. The sulfur atom can be derived from a sulfhydryl group of the targeting unit. Representative stretcher units of this embodiment include those within the square brackets of Formulas Xa and Xb, wherein the wavy line indicates attachment within the conjugate and R¹⁷ is —C₁-C₁₀ alkylene-, —C₁-C₁₀ heteroalkylene-, —C₃-C₈ carbocyclo-, —O—(C₁-C₈ alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkyl)C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—. —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁₀ alkylenearylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—. Any of the R¹⁷ substituents can be substituted or nonsubstituted. In an embodiment, the R¹⁷ substituents are unsubstituted. In another embodiment, the R¹⁷ substituents are optionally substituted. In some embodiments, the R¹⁷ groups are optionally substituted by a basic unit, e.g —(CH₂)ₓNH₂, —(CH₂)ₓNHR^{a}, and —(CH₂)ₓNR^{a}₂, wherein x is an integer in the range of 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group.

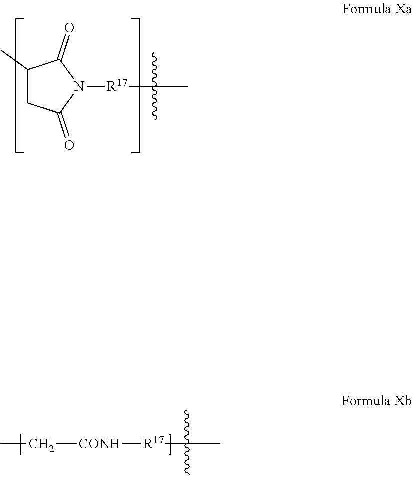

In the context of the embodiments of the stretcher unit, the wavy line may (although not necessarily) indicate attachment within the conjugate to either R₇, L₁, or Sₚ, whichever present. The free bond without the wavy line, typically at the opposite end of the stretcher unit, may indicate the bond to the targeting unit.

An illustrative stretcher unit is that of Formula Xa wherein R¹⁷ is —C₂-C₅ alkylene-C(═O)— wherein the alkylene is optionally substituted by a basic unit, e.g —(CH₂)ₓ—NH₂, —(CH₂)ₓNHR^{a}, and —(CH₂)ₓNR^{a}₂, wherein x is an integer in the range of 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group. Exemplary embodiments are as follows:

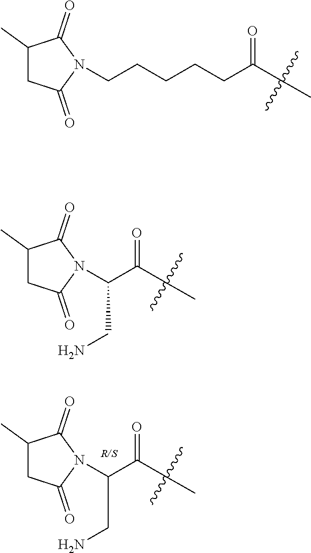 [SMILES: *C(=O)CCCCCN1C(=O)CC(C)C1=O ; *C(=O)[C@H](CN)N1C(=O)CC(C)C1=O ; *C(=O)C(CN)N1C(=O)CC(C)C1=O]

It will be understood that the substituted succinimide may exist in a hydrolyzed form as shown below:

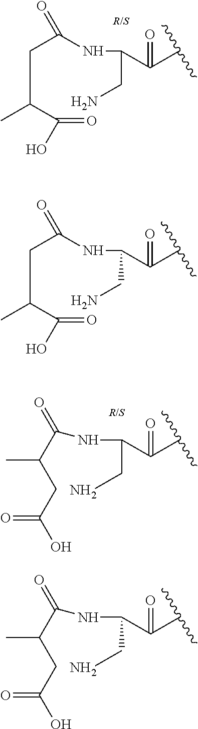

Illustrative stretcher units prior to conjugation to the targeting unit include the following:

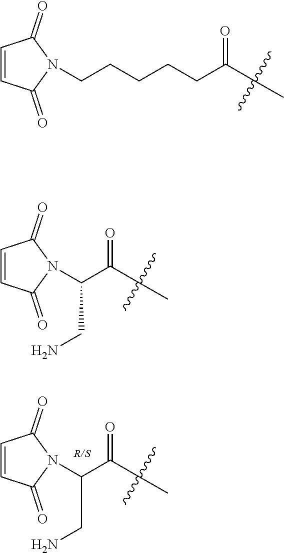 [SMILES: *C(=O)CCCCCN1C(=O)C=CC1=O ; *C(=O)[C@H](CN)N1C(=O)C=CC1=O ; *C(=O)C(CN)N1C(=O)C=CC1=O]

It will be understood that the amino group of the stretcher unit may be suitably protected by a amino protecting group during synthesis, e.g., an acid labile protecting group (e.g, BOC).

Yet another illustrative stretcher unit is that of Formula Xb wherein R¹⁷ is —(CH₂)₅—:

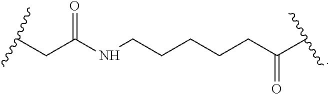 [SMILES: *CC(=O)NCCCCCC(*)=O]

In another embodiment, the stretcher unit is linked to the targeting unit via a disulfide bond between a sulfur atom of the targeting unit and a sulfur atom of the stretcher unit. A representative stretcher unit of this embodiment is depicted within the square brackets of Formula XI, wherein the wavy line indicates attachment within the conjugate and R¹⁷ is as described above for Formula Xa and Xb.

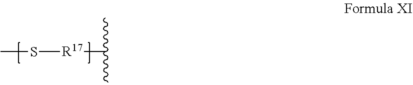

In yet another embodiment, the reactive group of the stretcher unit contains a reactive site that can form a bond with a primary or secondary amino group of the targeting unit. Example of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative stretcher units of this embodiment are depicted within the square brackets of Formulas XIIa, XIIb, and XIIc wherein the wavy line indicates attachment within the within the conjugate and R¹⁷ is as described above for Formula Xa and Xb.

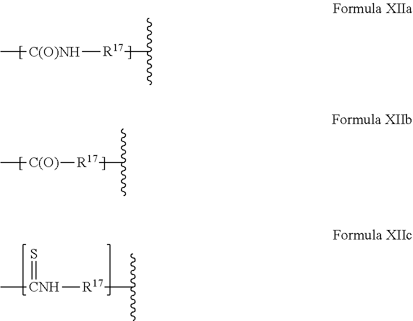

In yet another embodiment, the reactive group of the stretcher unit contains a reactive site that is reactive to a modified carbohydrate's (—CHO) group that can be present on the targeting unit. For example, a carbohydrate can be mildly oxidized using a reagent such as sodium periodate and the resulting (—CHO) unit of the oxidized carbohydrate can be condensed with a stretcher unit that contains a functionality such as a hydrazide, an oxime, a primary or secondary amine, a hydrazine, a thiosemicarbazone, a hydrazine carboxylate, and an arylhydrazide. Representative stretcher units of this embodiment are depicted within the square brackets of Formulas XIIIa, XIIIb, and XIIIc, wherein the wavy line indicates attachment within the conjugate and R¹⁷ is as described above for Formula Xa and Xb.

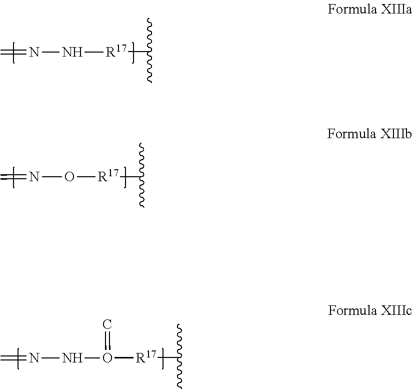

In some embodiments, it may be desirable to extend the length of the stretcher unit. Accordingly, a stretcher unit can comprise additional components. For example, a stretcher unit can include those within the square brackets of formula XIVa1:

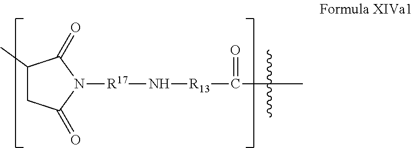

wherein the wavy line indicates attachment to the remainder of the conjugate and the free bond to the targeting unit;

and R¹⁷ is as described above. For example, R¹⁷ may be —C₂-C₅ alkylene-C(═O)— wherein the alkylene is optionally substituted by a basic unit, e.g —(CH₂)ₓNH₂, —(CH₂)ₓNHR^{a}, and —(CH₂)ₓNR^{a}₂, wherein x is an integer in the range of 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group; and

R¹³ is —C₁-C₆ alkylene-, —C₃-C₈ carbocyclo-, -arylene-, —C₁-C₁₀ heteroalkylene-, —C₃-C₈ heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene(C₃-C₈ heterocyclo)-, or —(C₃-C₈ heterocyclo)-C₁-C₁₀ alkylene-. In an embodiment, R¹³ is —C₁-C₆ alkylene-.

The stretcher unit may, in some embodiments, have a mass of no more than about 1000 daltons, no more than about 500 daltons, no more than about 200 daltons, from about 30, 50 or 100 daltons to about 1000 daltons, from about 30, 50 or 100 daltons to about 500 daltons, or from about 30, 50 or 100 daltons to about 200 daltons.

In an embodiment, the stretcher unit forms a bond with a sulfur atom of the targeting unit, for example an antibody. The sulfur atom can be derived from a sulfhydryl group of the antibody. Representative stretcher units of this embodiment are depicted within the square brackets of Formulas XVa and XVb, wherein R¹⁷ is selected from —C₁-C₁₀ alkylene-, —C₁-C₁₀ alkenylene-, —C₁-C₁₀ alkynylene-, carbocyclo-, —O—(C₁-C₈ alkylene)-, —O—(C₁-C₈ alkenylene)-, —O—(C₁-C₈ alkynylene)-, -arylene-, —C₁-C₁₀ alkylene-arylene-, —C₂-C₁₀ alkenylene-arylene, —C₂-C₁₀ alkynylene-arylene, -arylene-C₁-C₁₀ alkylene-, -arylene-C₂-C₁₀ alkenylene-, -arylene-C₂-C₁₀ alkynylene-, —C₁-C₁₀ alkylene-(carbocyclo)-, —C₂-C₁₀ alkenylene(carbocyclo)-, —C₂-C₁₀ alkynylene-(carbocyclo)-, -(carbocycle)-C₁-C₁₀ alkylene-, -(carbocycle)-C₂-C₁₀ alkenylene-, -(carbocyclo)-C₂-C₁₀ alkynylene, -heterocyclo-, —C₁-C₁₀ alkylene-(heterocyclo)-, —C₂-C₁₀ alkenylene-(heterocyclo)-, —C₂-C₁₀ alkynylene-(heterocyclo), -(heterocyclo)-C₁-C₁₀ alkylene-, -(heterocyclo)-C₂-C₁₀ alkenylene-, -(heterocyclo)-C₁-C₁₀ alkynylene-, —(CH₂CH₂O)ᵣ—, or —(CH₂CH₂O)ᵣ—CH₂—, and r is an integer ranging from 1-10, wherein said alkyl, alkenyl, alkynyl, alkylene, alkenylene, alkynyklene, aryl, carbocycle, carbocyclo, heterocyclo, and arylene radicals, whether alone or as part of another group, are optionally substituted. In some embodiments, said alkyl, alkenyl, alkynyl, alkylene, alkenylene, alkynyklene, aryl, carbocyle, carbocyclo, heterocyclo, and arylene radicals, whether alone or as part of another group, are unsubstituted. In some embodiments, R¹⁷ is selected from —C₁-C₁₀ alkylene-, -carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene(carbocyclo)-, -(carbocyclo)-C₁-C₁₀ alkylene-, —C₃-C₈ heterocyclo, —C₁-C₁₀ alkylene-(heterocyclo)-, -(heterocyclo)-C₁-C₁₀ alkylene-, —(CH₂CH₂O)ᵣ—, and —(CH₂CH₂O)ᵣ—CH₂—; and r is an integer ranging from 1-10, wherein said alkylene groups are unsubstituted and the remainder of the groups are optionally substituted.

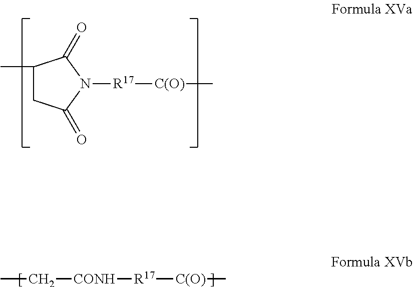

It is to be understood from all the exemplary embodiments that even where not denoted expressly, one or more glycosylation inhibitor moieties can be linked to a targeting unit, i.e. n may be 1 or more.

An illustrative stretcher unit is that of Formula XVa wherein R¹⁷ is —(CH₂CH₂O)ᵣ—CH₂—; and r is 2:

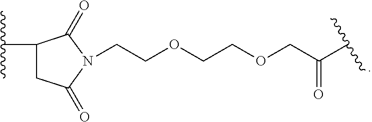 [SMILES: *C(=O)COCCOCCN1C(=O)CC(*)C1=O]

An illustrative stretcher unit is that of Formula XVa wherein R¹⁷ is arylene- or arylene-C₁-C₁₀ alkylene-. In some embodiments, the aryl group is an unsubstituted phenyl group.

In certain embodiments, the stretcher unit is linked to the targeting unit via a disulfide bond between a sulfur atom of the targeting unit and a sulfur atom of the stretcher unit. A representative stretcher unit of this embodiment is depicted in Formula XVI, wherein R¹⁷ is as defined above.

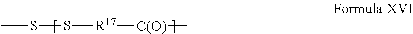

The S moiety in the formula XVI above may refer to a sulfur atom of the targeting unit, unless otherwise indicated by context.

In yet other embodiments, the stretcher unit contains a reactive site that can form a bond with a primary or secondary amino group of the targeting unit, such as an antibody. Examples of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative stretcher units of this embodiment are depicted within the square brackets of Formulas XVIIa and XVIIb, wherein —R¹⁷ is as defined above:

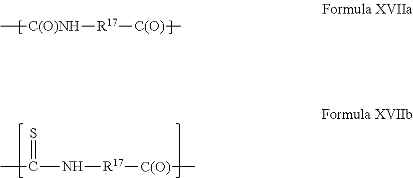

In some embodiments, the stretcher unit contains a reactive site that is reactive to a modified carbohydrate's (—CHO) group that can be present on the targeting unit, for example an antibody. For example, a carbohydrate can be mildly oxidized using a reagent such as sodium periodate and the resulting (—CHO) unit of the oxidized carbohydrate can be condensed with a stretcher unit that contains a functionality such as a hydrazide, an oxime, a primary or secondary amine, a hydrazine, a thiosemicarbazone, a hydrazine carboxylate, and an arylhydrazide. Representative stretcher units of this embodiment are depicted within the square brackets of Formulas XVIIIa, XVIIIb, and XVIIIc, wherein —R¹⁷— is as defined as above.

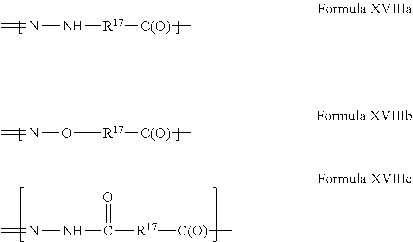

In embodiments in which the targeting unit is a glycoprotein, for example an antibody, the glycoprotein, i.e. the targeting unit, may be contacted with a suitable substrate, such as UDP-GalNAz, in the presence of a GalT or a GalT domain catalyst, for example a mutant GalT or GalT domain. Thus the targeting unit may have a GalNAz residue incorporated therein. The glycosylation inhibitor may then be conjugated via a reaction with the GalNAz thus incorporated in the targeting unit.

WO/2007/095506, WO/2008/029281 and WO/2008/101024 disclose methods of forming a glycoprotein conjugate wherein the glycoprotein is contacted with UDP-GalNAz in the presence of a GalT mutant, leading to the incorporation of GalNAz at a terminal non-reducing GlcNAc of an antibody carbohydrate. Subsequent copper-catalyzed or copper-free (metal-free) click chemistry with a terminal alkyne or Staudinger ligation may then be used to conjugate a molecule of interest, in this case the glycosylation inhibitor, optionally via a suitable linker unit or stretcher unit, to the attached azide moiety.

If no terminal GlcNAc sugars are present on the targeting unit, such as an antibody, endoenzymes Endo H, Endo A, Endo F, Endo D, Endo T, Endo S and/or Endo M and/or a combination thereof, the selection of which depends on the nature of the glycan, may be used to generate a truncated chain which terminates with one N-acetylglucosamine residue attached in an antibody Fc region.

In an embodiment, the endoglycosidase is Endo S, Endo S49, Endo F or a combination thereof.

In an embodiment, the endoglycosidase is Endo S, Endo F or a combination thereof.

Endo S, Endo A, Endo F, Endo M, Endo D and Endo H are known to the person skilled in the art. Endo S49 is described in WO/2013/037824 (Genovis AB). Endo S49 is isolated from Streptococcus pyogenes NZ131 and is a homologue of Endo S. Endo S49 has a specific endoglycosidase activity on native IgG and cleaves a larger variety of Fc glycans than Endo S.

Galactosidases and/or sialidases can be used to trim galactosyl and sialic acid moieties, respectively, before attaching e.g. GalNAz moieties to terminal GlcNAcs. These and other deglycosylation steps, such as defucosylation, may be applied to G2F, G1F, G0F, G2, G1, and G0, and other glycoforms.

Mutant GalTs include but are not limited to bovine beta-1,4-galactosyltransferase I (GalT1) mutants Y289L, Y289N, and Y289I disclosed in Ramakrishnan and Qasba, J. Biol. Chem., 2002, vol. 277, 20833) and GalT1 mutants disclosed in WO/2004/063344 and WO/2005/056783 and their corresponding human mutations.

Mutant GalTs (or their GalT domains) that catalyze the formation of i) a glucose-β(1,4)-N-acetylglucosamine bond, ii) an N-acetylgalactosamine-β(1,4)-N-acetylglucosamine bond, iii) a N-acetylglucosamine-β(1,4)-N-acetylglucosamine bond, iv) a mannose-β(1,4)-N-acetylglucosamine bond are disclosed in WO 2004/063344. The disclosed mutant GalT (domains) may be included within full-length GalT enzymes, or in recombinant molecules containing the catalytic domains, as disclosed in WO2004/063344.

In an embodiment, GalT or GalT domain is for example Y284L, disclosed by Bojarová et al., Glycobiology 2009, 19, 509.

In an embodiment, GalT or GalT domain is for example R228K, disclosed by Qasba et al., Glycobiology 2002, 12, 691.

In an embodiment, the mutant GalT1 is a bovine β(1,4)galactosyltransferase 1.

In an embodiment, the bovine GalT1 mutant is selected from the group consisting of Y289L, Y289N, Y289I, Y284L and R228K.

In an embodiment, the mutant bovine GalT1 or GalT domain is Y289L.

In an embodiment, the GalT comprises a mutant GalT catalytic domain from a bovine β(1,4)-galactosyltransferase, selected from the group consisting of GalT Y289F, GalT Y289M, GalT Y289V, GalT Y289G, GalT Y289I and GalT Y289A. These mutants may be provided via site-directed mutagenesis processes, in a similar manner as disclosed in WO 2004/063344, in Qasba et al., Prot. Expr. Pur. 2003, 30, 219 and in Qasba et al., J. Biol. Chem. 2002, 277, 20833 for Y289L, Y289N and Y289I.

Another type of a suitable GalT is α(1,3)-N-galactosyltransferase (α3Gal-T).

In an embodiment, α(1,3)-N-acetylgalactosaminyltransferase is α3GalNAc-T as disclosed in

WO2009/025646. Mutation of α3Gal-T can broaden donor specificity of the enzyme, and make it an α3GalNAc-T. Mutation of α3GalNAc-T can broaden donor specificity of the enzyme. Polypeptide fragments and catalytic domains of α(1,3)-N-acetylgalactosaminyltransferases are disclosed in WO/2009/025646.

In an embodiment, the GalT is a wild-type galactosyltransferase.

In an embodiment, the GalT is a wild-type β(1,4)galactosyltransferase or a wild-type β(1,3)-N-galactosyltransferase.

In an embodiment, GalT is β(1,4)-galactosyltransferase I.

In an embodiment, the β(1,4)-galactosyltransferase is selected from the group consisting of a bovine β(1,4)-Gal-T1, a human β(1,4)-Gal-T1, a human β(1,4)-Gal-T2, a human β(1,4)-Gal-T3, a human β(1,4)-Gal-T4 and β(1,3)-Gal-T5.

In an embodiment, β-(1,4)-N-acetylgalactosaminyltransferase is selected from the mutants disclosed in WO 2016/170186.

The linker unit or the stretcher unit may comprise an alkyne group, for example a cyclic alkyne group, capable of reacting with the azide group of the GalNAz incorporated in the targeting unit, thereby forming a triazole group. Examples of suitable cyclic alkyne groups may include DBCO, OCT, MOFO, DIFO, DIFO2, DIFO3, DIMAC, DIBO, ADIBO, BARAC, BCN, Sondheimer diyne, TMDIBO, S-DIBO, COMBO, PYRROC, or any modifications or analogs thereof.

BCN and its derivatives are disclosed in WO/2011/136645. DIFO, DIFO2 and DIFO 3 are disclosed in US 2009/0068738. DIBO is disclosed in WO 2009/067663. DIBO may optionally be sulfated (S-DIBO) as disclosed in J. Am. Chem. Soc. 2012, 134, 5381. BARAC is disclosed in J. Am. Chem. Soc. 2010, 132, 3688-3690 and US 2011/0207147. ADIBO derivatives are disclosed in WO/2014/189370. The stretcher unit may thus comprise an optionally substituted triazole group formed by a reaction between a (cyclic) alkyne group and an azide group of a GalNAz group incorporated at a terminal non-reducing GlcNAc of the targeting unit.

VI) Specificity Units

The term “specificity unit” or Sₚ may refer to any group, moiety or linker portion capable of linking R₇ or L₁ (if present) to L₂ (if present), to R₈ (if present) or to the targeting unit.

The specificity unit may, in some embodiments, be cleavable. Thereby it can confer cleavability to the linker unit.

The specificity unit may comprise a labile bond configured to be cleavable in suitable conditions. It may thus confer specificity to the cleavability of the conjugate. For example, the stretcher unit may be cleavable only after the cleavage of the specificity unit.

The specificity unit can be, for example, a monopeptide, dipeptide, tripeptide, tetrapeptide, pentapeptide, hexapeptide, heptapeptide, octapeptide, nonapeptide, decapeptide, undecapeptide or dodecapeptide unit. Each Sₚ unit independently may have the formula XIXa or XIXb denoted below in the square brackets:

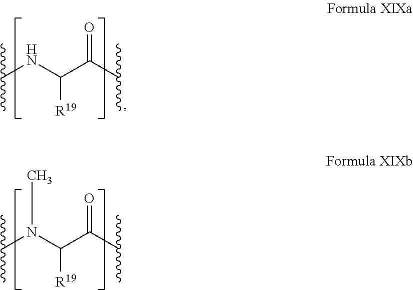

wherein R¹⁹ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, (CH₂)₄NH₂, (CH₂)₄NHCOCH₃, —(CH₂)₄NHCHO, —(CH₂)₃NHCONH₂, (CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

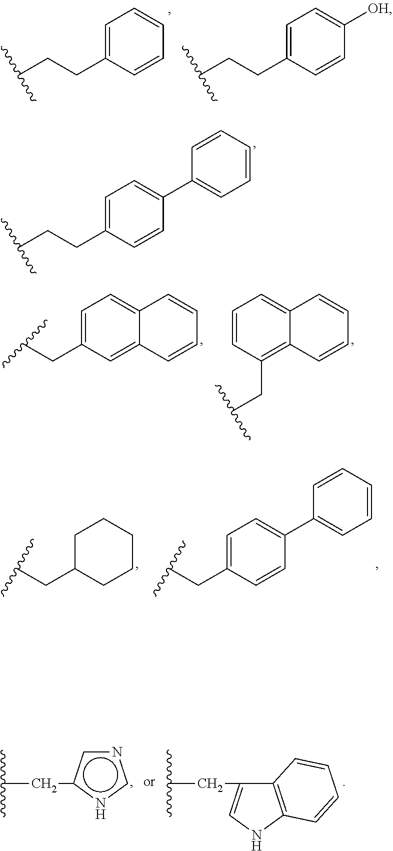

In some embodiments, the specificity unit can be enzymatically cleavable by one or more enzymes, including a cancer or tumor-associated protease, to liberate the glycosylation inhibitor.

In certain embodiments, the specificity unit can comprise natural amino acids. In other embodiments, the specificity unit can comprise non-natural amino acids. Illustrative specificity units are represented by formulas (XX)-(XXII):

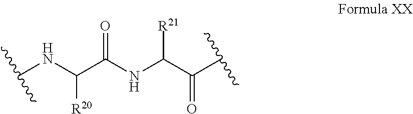

wherein R²⁰ and R²¹ are as follows:

| R²⁰ | R²¹ |
|---|---|
| Benzyl | (CH₂)₄NH₂; |
| methyl | (CH₂)₄NH₂; |
| isopropyl | (CH₂)₄NH₂; |
| isopropyl | (CH₂)₃NHCONH₂; |
| benzyl | (CH₂)₃NHCONH₂; |
| isobutyl | (CH₂)₃NHCONH₂; |
| sec-butyl | (CH₂)₃NHCONH₂; |
| 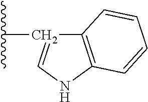 [SMILES: *c1c[nH]c2ccccc12] | (CH₂)₃NHCONH₂; |
| benzyl | methyl; |
| benzyl | (CH₂)₃NHC(═NH)NH₂ ; |

Formula XXI

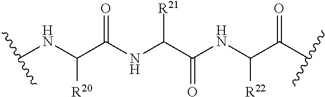

wherein R²⁰, R²¹ and R²² are as follows:

| R²⁰ | R²¹ | R²² |
|---|---|---|
| benzyl | benzyl | (CH₂)₄NH₂; |
| isopropyl | benzyl | (CH₂)₄NH₂; and |
| H | benzyl | (CH₂)₄NH₂ |

Formula XXII

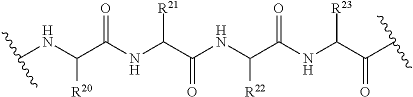

wherein R²⁰, R²¹, R²² and R²³ are as follows:

| R²⁰ | R²¹ | R²² | R²³ |
|---|---|---|---|
| H | benzyl | isobutyl | H; and |
| methyl | isobutyl | methyl | isobutyl |

Exemplary specificity units include, but are not limited to, units of formula XX wherein R²⁰ is benzyl and R²¹ is —(CH₂)₄NH₂; R²⁰ is isopropyl and R₂₁ is —(CH₂)₄NH₂; or R²⁰ is isopropyl and R₂₁ is —(CH₂)₃NHCONH₂. Another exemplary specificity unit is a specificity unit of formula XXI wherein R²⁰ is benzyl, R²¹ is benzyl, and R²² is —(CH₂)₄NH₂.

Useful specificity units can be designed and optimized in their selectivity for enzymatic cleavage by a particular enzyme, for example, a tumour-associated protease. In one embodiment, the specificity unit is cleavable by cathepsin B, C and D, or a plasmin protease.

In an embodiment, the specificity unit is a dipeptide, tripeptide, tetrapeptide or pentapeptide. When R¹⁹, R²⁰, R²¹, R²² or R²³ is other than hydrogen, the carbon atom to which R¹⁹, R²⁰, R²¹, R²² or R²³ is attached is chiral. Each carbon atom to which R¹⁹, R²⁰, R²¹, R²² or R²³ is attached may be independently in the (S) or (R) configuration.

In an embodiment, the specificity unit comprises or is valine-citrulline (vc or val-cit). In another embodiment, the the specificity unit unit is phenylalanine-lysine (i.e. fk). In yet another embodiment, the specificity unit comprises or is N-methylvaline-citrulline. In yet another embodiment, the specificity unit comprises or is 5-aminovaleric acid, homo phenylalanine lysine, tetraisoquinolinecarboxylate lysine, cyclohexylalanine lysine, isonepecotic acid lysine, beta-alanine lysine, glycine serine valine glutamine and isonepecotic acid.

VII) Spacer Units

The term “spacer unit” may refer to any group, moiety or linker portion capable of linking R₇ to Sₚ (if present), L₂ (if present) or the targeting unit. Various types of spacer units may be suitable, and many are known in the art.

Spacer units may be of two general types: non self-immolative or self-immolative. A non self-immolative spacer unit is one in which part or all of the spacer unit remains bound to the glycosylation inhibitor moiety after cleavage, for example enzymatic cleavage, of a specificity unit from the conjugate. Examples of a non self-immolative spacer unit include, but are not limited to a (glycine-glycine) spacer unit and a glycine spacer unit. When a conjugate containing a glycine-glycine spacer unit or a glycine spacer unit undergoes enzymatic cleavage via an enzyme (e.g., a tumour-cell associated-protease, a cancer-cell-associated protease or a lymphocyte-associated protease), a glycine-glycine-R₇-glycosylation inhibitor moiety or a glycine-R₇-glycosylation inhibitor moiety is cleaved from —Sₚ-L₂-R₈-T (whichever, if any, of Sₚ-L₂-R₈ is present). In one embodiment, an independent hydrolysis reaction takes place within the target cell, cleaving the glycine-R₇-glycosylation inhibitor moiety bond and liberating the glycosylation inhibitor (and R₇).

In some embodiments, the non self-immolative spacer unit (-L₁-) is -Gly-. In some embodiments, the non self-immolative spacer unit (-L₁-) is -Gly-Gly-.

However, the spacer unit may also be absent.

Alternatively, a conjugate containing a self-immolative spacer unit can release -D, i.e. the glycosylation inhibitor, or D-R₇—. In the context of this specification, the term “self-immolative spacer unit” may refer to a bifunctional chemical moiety that is capable of covalently linking together two spaced chemical moieties into a stable tripartite molecule. It may spontaneously separate from the second chemical moiety if its bond to the first moiety is cleaved.

In some embodiments, the spacer unit is a p-aminobenzyl alcohol (PAB) unit (see Schemes 1 and 2 below) the phenylene portion of which is substituted with Qₘ wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

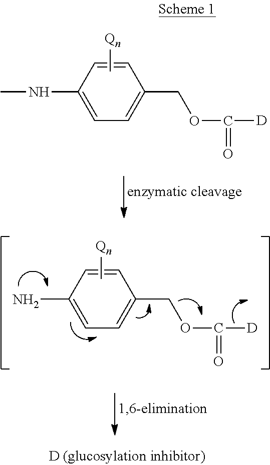

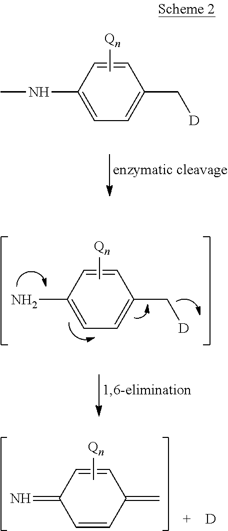

In some embodiments, the spacer unit is a PAB group that is linked to —Sᵣ—, -L₂-, —R₈— or -T via the amino nitrogen atom of the PAB group, and connected directly to —R₇— or to -D via a carbonate, carbamate or ether group. Without being bound by any particular theory or mechanism, Scheme 1 depicts a possible mechanism of release of a PAB group which is attached directly to -D or R⁷ via a carbamate or carbonate group.

In Scheme 1, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0 4. The alkyl, alkenyl and alkynyl groups, whether alone or as part of another group, can be optionally substituted.

Without being bound by any particular theory or mechanism, Scheme 2 depicts a possible mechanism of glycosylation inhibitor release of a PAB group which is attached directly to -D or to —R₇-D via an ether or amine linkage, wherein D may include the oxygen or nitrogen group that is part of the glycosylation inhibitor.

In Scheme 2, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

Other examples of self-immolative spacer units include, but are not limited to, aromatic compounds that are electronically similar to the PAB group such as 2-aminoimidazol-5-methanol derivatives and ortho or para-aminobenzylacetals. Other possible spacer units may be those that undergo cyclization upon amide bond hydrolysis, such as substituted and unsubstituted 4-aminobutyric acid amides, appropriately substituted bicyclo[2.2.1] and bicyclo[2.2.2] ring systems and 2-aminophenylpropionic acid amides. Elimination of amine-containing glycosylation inhibitors that are substituted at the α-position of glycine are also examples of self-immolative spacers.

In an embodiment, the spacer unit is a branched bis(hydroxymethyl)-styrene (BHMS) unit as depicted in Scheme 3, which can be used to incorporate and release multiple glycosylation inhibitors.

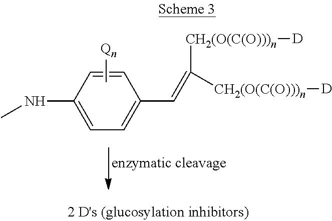

In Scheme 3, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; and n is 0 or 1. The alkyl, alkenyl and alkynyl groups, whether alone or as part of another group, can be optionally substituted.

In some embodiments, the -D moieties are the same. In yet another embodiment, the -D moieties are different.

In an embodiment, the spacer unit is represented by any one of Formulas (XXIII)-(XXV):

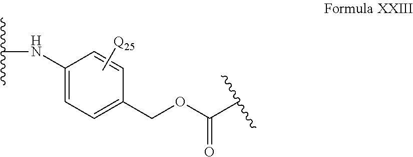

wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted;

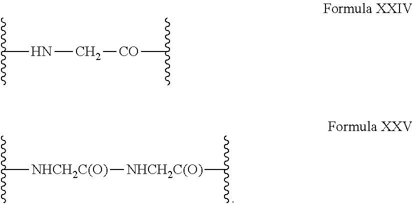

VIII) Further Linker Units

The linker unit may, in some embodiments, comprise a polymer moiety. Such polymer moieties are described e.g. in WO 2015/189478.

In an embodiment, the linker unit L comprises a moiety represented by the formula XXVI, or L is represented by the formula XXVI:

—Y—(CH₂)ₒ—O]_{q}—P— Formula XXVI

wherein

P is a polymer selected from the group consisting of dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin and carrageenan, or a derivative thereof;

o is in the range of 1 to 10;

q is at least 1; and

each Y is independently selected from the group consisting of S, NH and 1,2,3-triazolyl, wherein 1,2,3-triazolyl is optionally substituted.

In the above formula, P may be linked to T and Y to D, i.e. the glycosylation inhibitor. Y may be linked to D directly, or further groups, moieties or units may be present between Y and D.

Dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin and carrageenan each comprise at least one hydroxyl group. The presence of the at least one hydroxyl group allows the linking of one or more substituents to the polymer as described herein. Many of these polymers also comprise saccharide units that may be further modified, e.g. oxidatively cleaved, to introduce functional groups to the polymer. P may thus also be a polymer derivative.

In this specification, the term “saccharide unit” should be understood as referring to a single monosaccharide moiety.

In this specification, the term “saccharide” should be understood as referring to a monosaccharide, disaccharide or an oligosaccharide.

The value of q may depend e.g. on the polymer, on the glycosylation inhibitor, the linker unit, and the method of preparing the conjugate. Typically, a large value of q may led to higher efficiency of the conjugate; on the other hand, a large value of q may in some cases affect other properties of the conjugate, such as pharmacokinetic properties or solubility, adversely. In an embodiment, q is in the range of 1 to about 300, or in the range of about 10 to about 200, or in the range of about 20 to about 100, or in the range of about 20 to about 150. In an embodiment, q is in the range of 1 to about 20, or in the range of 1 to about 15 or in the range of 1 to about 10. In an embodiment, q is 1, 2, 3, 4, 5, 6, 7, 8, 9, 10, 11, 12, 13, 14, 15, 16, 17, 18, 19, or 20. In an embodiment, q is 2-16. In an embodiment, q is in the range of 2 to 10. In other embodiments, q is in the range of 2 to 6; 2 to 5; 2 to 4; 2 or 3; or 3 or 4.

In an embodiment, about 25-45% of carbons of the polymer bearing a hydroxyl group are substituted by a substituent of the formula D-Y—(CH₂)ₙ—O—.

In embodiments in which the polymer comprises a plurality of saccharide units, the ratio of q to the number of saccharide units of the polymer may be e.g. 1:20 to 1:3 or 1:4 to 1:2.

In an embodiment, o is 1, 2, 3, 4, 5, 6, 7, 8, 9 or 10. In an embodiment, o is in the range of 2 to 9, or in the range of 3 to 8, or in the range of 4 to 7, or in the range of 1 to 6, or in the range of 2 to 5, or in the range of 1 to 4.

Each o may, in principle, be independently selected. Each o in a single conjugate may also be the same.

In an embodiment, Y is S.

In an embodiment, Y is NH.

In an embodiment, Y is 1,2,3-triazolyl. In this specification, the term “1,2,3-triazolyl” should be understood as referring to 1,2,3-triazolyl, or to 1,2,3-triazolyl which is substituted. In an embodiment, the 1,2,3-triazolyl is a group formed by click conjugation comprising a triazole moiety. Click conjugation should be understood as referring to a reaction between an azide and an alkyne yielding a covalent product—1,5-disubstituted 1,2,3-triazole—such as copper(I)-catalysed azide-alkyne cycloaddition reaction (CuAAC). Click conjugation may also refer to copper-free click chemistry, such as a reaction between an azide and a cyclic alkyne group such as dibenzocyclooctyl (DBCO). “1,2,3-triazolyl” may thus also refer to a group formed by a reaction between an azide and a cyclic alkyne group, such as DBCO, wherein the group comprises a 1,2,3-triazole moiety.

In an embodiment, the linker unit L comprises a moiety represented by the formula XXVII, or L is represented by the formula XXVII

—Y′—(CH₂)ₚ—S—(CH₂)ₒ—O]_{q}—P— Formula XXVII

wherein

P is a polymer selected from the group consisting of dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin and carrageenan, or a derivative thereof;

q is at least 1;

o is in the range of 1 to 10;

p is in the range of 1 to 10; and

each Y′ is independently selected from the group consisting of NH and 1,2,3-triazolyl, wherein 1,2,3-triazolyl is optionally substituted.

In the context of Formula XXVII, each of P, o and q may be as defined for Formula XXVI.

In an embodiment, p is 3, 4, 5, 6, 7, 8, 9 or 10. In an embodiment, p is in the range of 3 to 4, or in the range of 3 to 5, or in the range of 3 to 6, or in the range of 3 to 7, or in the range of 3 to 8, or in the range of 3 to 9. Each p may, in principle, be independently selected. Each p in a single conjugate may also be the same.

In an embodiment, Y′ is selected from the group consisting of NH and 1,2,3-triazolyl.

In an embodiment, P is a polymer derivative comprising at least one saccharide unit.

In an embodiment, P is a polymer derivative comprising at least one saccharide unit, and the polymer derivative is bound to the targeting unit (for example, an antibody) via a bond formed by a reaction between at least one aldehyde group formed by oxidative cleavage of a saccharide unit of the polymer derivative and an amino group of the targeting unit.

In an embodiment, the saccharide unit is a D-glucosyl, D-mannosyl, D-galactosyl, L-fucosyl, D-N-acetylglucosaminyl, D-N-acetylgalactosaminyl, D-glucuronidyl, or D-galacturonidyl unit, or a sulphated derivative thereof.

In an embodiment, the D-glucosyl is D-glucopyranosyl.

In an embodiment, the polymer is selected from the group consisting of dextran, mannan, pullulan, hyaluronic acid, hydroxyethyl starch, chondroitin sulphate, heparin, heparin sulphate, amylose, amylopectin, chitosan, cyclodextrin, pectin and carrageenan. These polymers have the added utility that they may be oxidatively cleaved so that aldehyde groups are formed.

In an embodiment, the polymer is dextran.

In this specification, “dextran” should be understood as referring to a branched glucan composed of chains of varying lengths, wherein the straight chain consists of a α-1,6 glycosidic linkages between D-glucosyl (D-glucopyranosyl) units. Branches are bound via α-1,3 glycosidic linkages and, to a lesser extent, via α-1,2 and/or α-1,4 glycosidic linkages. A portion of a straight chain of a dextran molecule is depicted in the schematic representation below.

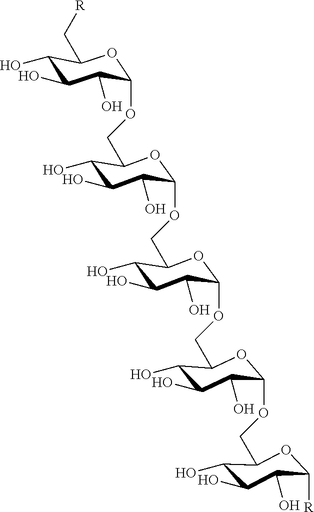

“D-glucosyl unit” should be understood as referring to a single D-glucosyl molecule. Dextran thus comprises a plurality of D-glucosyl units. In dextran, each D-glucosyl unit is bound to at least one other D-glucosyl unit via a α-1,6 glycosidic linkage, via a α-1,3 glycosidic linkage or via both.

Each D-glucosyl unit of dextran comprises 6 carbon atoms, which are numbered 1 to 6 in the schematic representation below. The schematic representation shows a single D-glucosyl unit bound to two other D-glucosyl units (not shown) via α-1,6 glycosidic linkages.

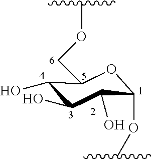

Carbons 2, 3 and 4 may be substituted by free hydroxyl groups. In D-glucosyl units bound to a second D-glucosyl unit via a α-1,3 glycosidic linkage, wherein carbon 3 of the D-glucosyl unit is bound via an ether bond to carbon 1 of the second D-glucosyl unit, carbons 2 and 4 may be substituted by free hydroxyl groups. In D-glucosyl units bound to a second D-glucosyl unit via a α-1,2 or α-1,4 glycosidic linkage, wherein carbon 2 or 4 of the D-glucosyl unit is bound via an ether bond to carbon 1 of the second D-glucosyl unit, carbons 3 and 4 or 2 and 3, respectively, may be substituted by free hydroxyl groups.

A skilled person will understand that other polymers described in this specification also contain free hydroxyl groups bound to one or more carbon atoms and have also other similar chemical properties.

Carbohydrate nomenclature is essentially according to recommendations by the IUPAC-IUB Commission on Biochemical Nomenclature (e.g. Carbohydrate Res. 1998, 312, 167; Carbohydrate Res. 1997, 297, 1; Eur. J. Biochem. 1998, 257, 293).

In this specification, the term “Ficoll” refers to an uncharged, highly branched polymer formed by the co-polymerisation of sucrose and epichlorohydrin.

In an embodiment, the polymer is a dextran derivative comprising at least one D-glucosyl unit;

o is in the range of 3 to 10;

Y is S;

the dextran derivative comprises at least one aldehyde group formed by oxidative cleavage of a D-glucosyl unit; and

the dextran derivative is bound to the targeting unit (for example, an antibody) via a bond formed by a reaction between at least one aldehyde group of the dextran and an amino group of the targeting unit.

Saccharide units of the polymer, for instance the D-glucosyl units of dextran, may be cleaved by oxidative cleavage of a bond between two adjacent carbons substituted by a hydroxyl group. The oxidative cleavage cleaves vicinal diols, such as D-glucosyl and other saccharide units in which two (free) hydroxyl groups occupy vicinal positions. Saccharide units in which carbons 2, 3 and 4 are substituted by free hydroxyl groups may thus be oxidatively cleaved between carbons 2 and 3 or carbons 3 and 4. Thus a bond selected from the bond between carbons 2 and 3 and the bond between carbons 3 and 4 may be oxidatively cleaved. D-glucosyl units and other saccharide units of dextran and other polymers may be cleaved by oxidative cleavage using an oxidizing agent such as sodium periodate, periodic acid and lead(IV) acetate, or any other oxidizing agent capable of oxidatively cleaving vicinal diols.

Oxidative cleavage of a saccharide unit forms two aldehyde groups, one aldehyde group at each end of the chain formed by the oxidative cleavage. In the conjugate, the aldehyde groups may in principle be free aldehyde groups. However, the presence of free aldehyde groups in the conjugate is typically undesirable.

Therefore the free aldehyde groups may be capped or reacted with an amino group of the targeting unit, or e.g. with a tracking molecule.

In an embodiment, the polymer derivative is bound to the targeting unit via a bond formed by a reaction between at least one aldehyde group formed by oxidative cleavage of a saccharide unit of the polymer derivative and an amino group of the targeting unit.

In an embodiment, the polymer derivative may also be bound to the targeting unit via a group formed by a reaction between at least one aldehyde group formed by oxidative cleavage of a saccharide unit of the polymer derivative and an amino group of the targeting unit.

The aldehyde group formed by oxidative cleavage readily reacts with an amino group in solution, such as an aqueous solution. The resulting group or bond formed may, however, vary and is not always easily predicted and/or characterised. The reaction between at least one aldehyde group formed by oxidative cleavage of a saccharide unit of the polymer derivative and an amino group of the targeting unit may result e.g. in the formation of a Schiff base. Thus the group via which the polymer derivative is bound to the targeting unit may be e.g. a Schiff base (imine) or a reduced Schiff base (secondary amine).

IX) Conjugates

In exemplary embodiments, the conjugate is represented by Formula C:

[D-R₇-L₁-Sₚ-L₂-R₈-]ₙ-T Formula C

wherein

D, R₇, L₁, Sₚ, L₂, R₈, n and T are selected from the embodiments described in Table 1.

TABLE 1
Exemplary conjugate units.

| | |
|---|---|
| Unit | Preferred embodiments |

| D, | a. | an N-acetylglucosaminylation |
|---|---|---|
| glycosylation | | inhibitor, |
| inhibitor | b. | 2-acetamido-2,4-dideoxy-4- |
| | | fluoroglucosamine, |
| | c. | peracetyl-2-acetamido-2,4- |
| | | dideoxy-4-fluoroglucosamine, |
| | d. | 2-acetamido-2,3-dideoxy-3- |
| | | fluoroglucosamine, |
| | e. | 2-acetamido-2,6-dideoxy-6- |
| | | fluoroglucosamine, |
| | f. | 4-deoxy-4-fluoroglucosamine, |
| | g. | 3-deoxy-3-fluoroglucosamine, |
| | h. | 6-deoxy-6-fluoroglucosamine, |
| | i. | a sialylation inhibitor, |
| | j. | 3-deoxy-3-fluorosialic acid, |
| | k. | peracetyl-3-deoxy-3-fluorosialic |
| | | acid, |
| | l. | 3-deoxy-3ax-fluorosialic acid, |
| | m. | 3-deoxy-3eq-fluorosialic acid, |
| | n. | 3-deoxy-3-fluoro-Neu5Ac, |
| | o. | 3-deoxy-3ax-fluoro-Neu5Ac, |
| | p. | peracetyl-3-deoxy-3ax-fluoro- |
| | | Neu5Ac, |
| | q. | 3-deoxy-3eq-fluoro-Neu5Ac |
| | r. | 3-deoxy-3-fluoro-Neu5N, |
| | s. | 3-deoxy-3ax-fluoro-Neu5N, |
| | t. | 3-deoxy-3eq-fluoro-Neu5N, |
| | u. | an N-glycosylation inhibitor, |
| | v. | tunicamycin, |
| | w. | an N-glycan processing inhibitor, |
| | x. | a mannosidase I inhibitor, |
| | y· | kifunensine |
| | z. | a hexosamine pathway inhibitor, |
| | aa. | a PGM3 inhibitor, or |
| | bb. | GlcNAc-thiazoline. |
| R₇, a group | a. | —C(═O)NH—, |
| covalently | b. | —C(═O)O—, |
| bonded to the | c. | —NHC(═O)—, |
| glycosylation | d. | —OC(═O)—, |
| inhibitor | e. | —OC(═O)O—, |
| | f. | —NHC(═O)O—, |
| | g. | —OC(═O)NH—, |
| | h. | —NHC(═O)NH, |
| | i. | —NH—, |
| | j. | —O—, |
| | k. | —S—, or |
| | l. | absent |
| L₁, a spacer | a. | a C₁₋₁₂ alkyl, |
| unit | b. | a substituted C₁₋₁₂ alkyl, |
| | c. | a C₅₋₂₀ aryl, |
| | d. | a substituted C₅₋₂₀ aryl, |
| | e. | a PEG₁₋₅₀ polyethylene glycol |
| | | moiety, |
| | f. | a substituted PEG₁₋₅₀ polyethylene |
| | | glycol moiety, |
| | g. | a branched PEG₂₋₅₀ polyethylene |
| | | glycol moiety, |
| | h. | a substituted branched PEG₂₋₅₀ |
| | | polyethylene glycol moiety, |
| | i. | a PAB group, or |
| | j. | absent |
| Sₚ, a | a. | dipeptide, |
| specificity | b. | tripeptide, |
| unit | c. | tetrapeptide, |
| | d. | valine-citrulline, |
| | e. | phenylalanine-lysine, |
| | f. | valine-alanine, |
| | g. | valine-serine, |
| | h. | a hydrazone, |
| | i. | an ester, |
| | j. | a disulfide, |
| | k. | a glycoside, or |
| | l. | absent |
| L₂, a | a. | a C₁₋₁₂ alkyl, |
| stretcher | b. | a substituted C₁₋₁₂ alkyl, |
| unit | c. | a C₅₋₂₀ aryl, |
| covalently | d. | a substituted C₅₋₂₀ aryl, |
| bonded to the | e. | a PEG₁₋₅₀ polyethylene glycol |
| targeting | | moiety, |
| unit | f. | a substituted PEG₁₋₅₀ polyethylene |
| | | glycol moiety, |
| | g. | a branched PEG₂₋₅₀ polyethylene |
| | | glycol moiety, |
| | h. | a substituted branched PEG₂₋₅₀ |
| | | polyethylene glycol moiety, |
| | i. | a moiety represented by the |
| | | formula XXVI, |
| | j· | a moiety represented by the |
| | | formula XXVII, or |
| | k. | absent |
| R₈, a group | a. | —C(═O)NH—, |
| covalently | b. | —C(═O)O—, |
| bonded to the | c. | —NHC(═O)—, |
| targeting | d. | —OC(═O)—, |
| unit | e. | —OC(═O)O—, |
| | f. | —NHC(═O)O—, |
| | g. | —OC(═O)NH—, |
| | h. | —NHC(═O)NH, |
| | i. | —NH—, |
| | j. | —O—, |
| | k. | —S—, or |
| | l. | absent |
| n, number of | about 1, 2, 3, 4, 6, 8, 10, 12, 14, 16, | |
| D-L moieties | 18, 20, 22, 24, 28, 30, 32, 36, 40, 44, | |
| per targeting | 48, 56, 64, 72, 80, 90, or 100 | |
| unit | | |
| T, targeting | a. | antibody, or |
| unit | b. | peptide |

The conjugate may be any conjugate described in this specification; a skilled person may derive various conjugates by combining any one of the above units and glycosylation inhibitors described in this specification.

The conjugate may be selected from the group consisting of conjugates represented by formulas Va-c, VIa-b, VIIa-b or VIIIa-t:

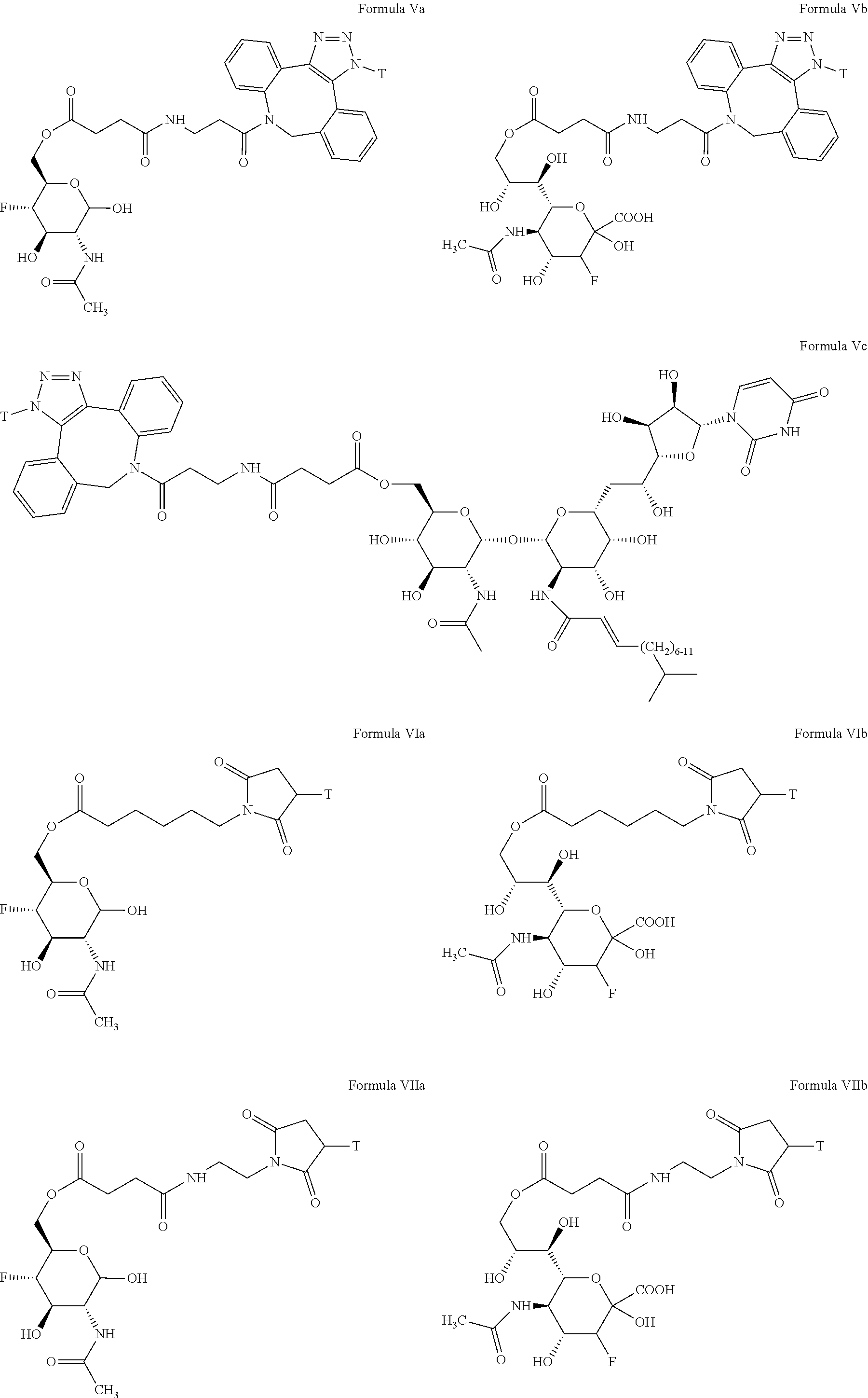

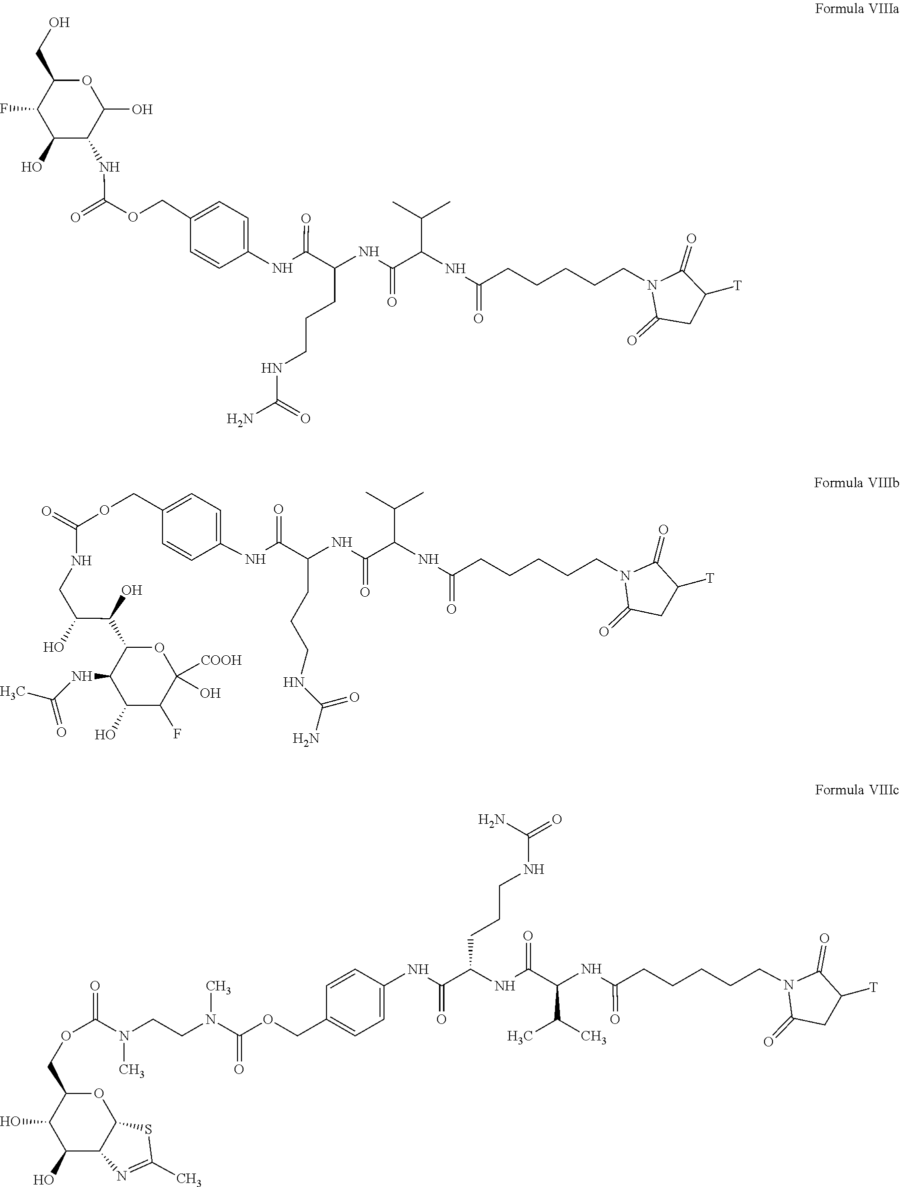

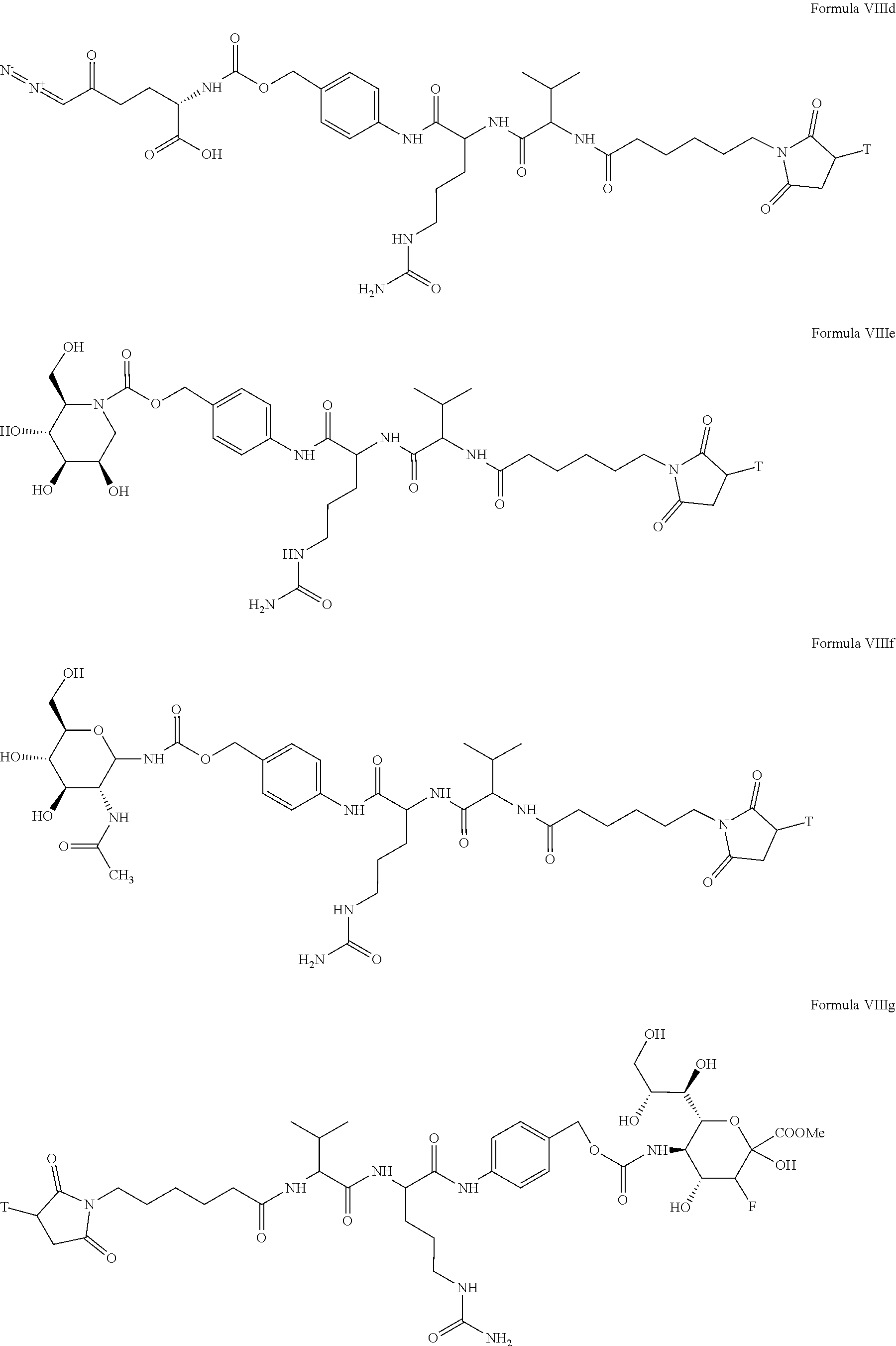

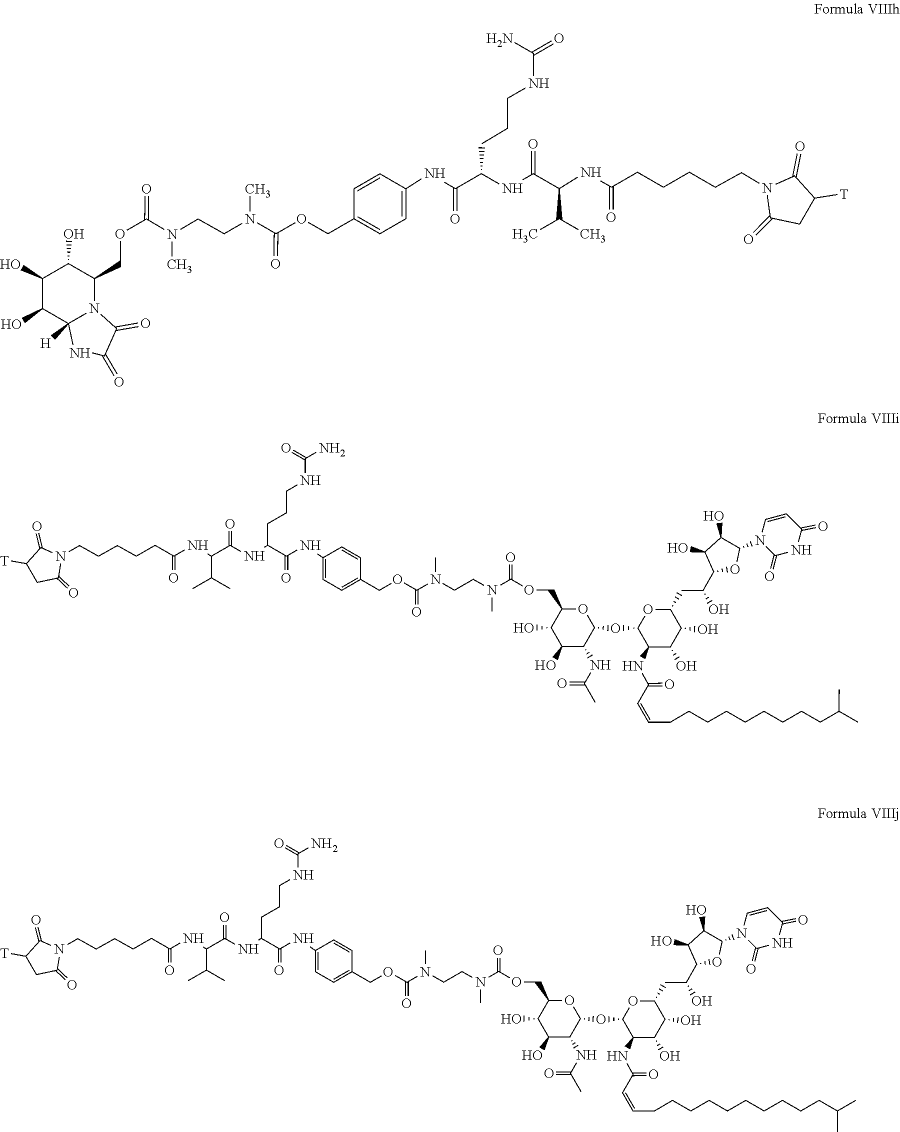

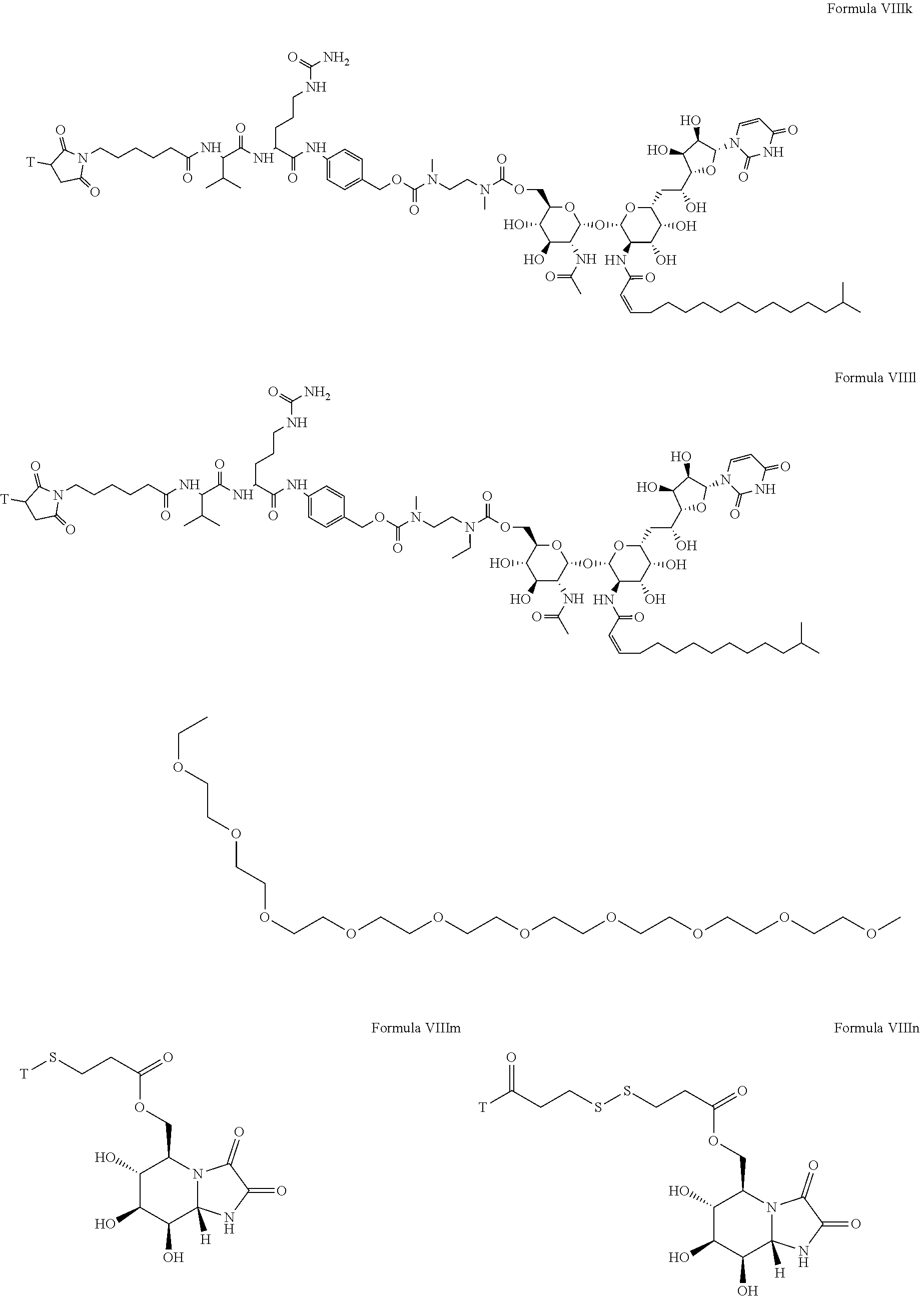

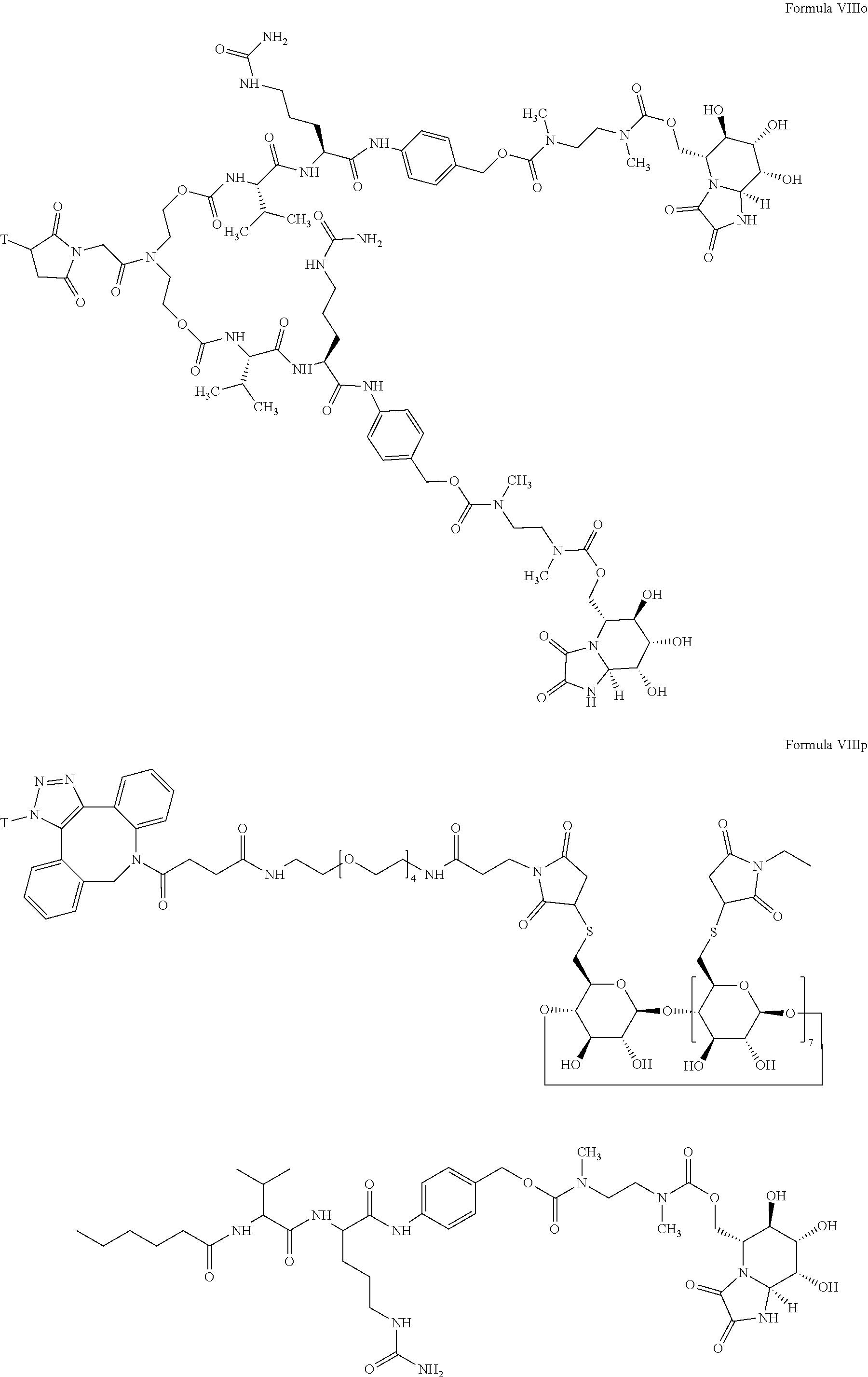

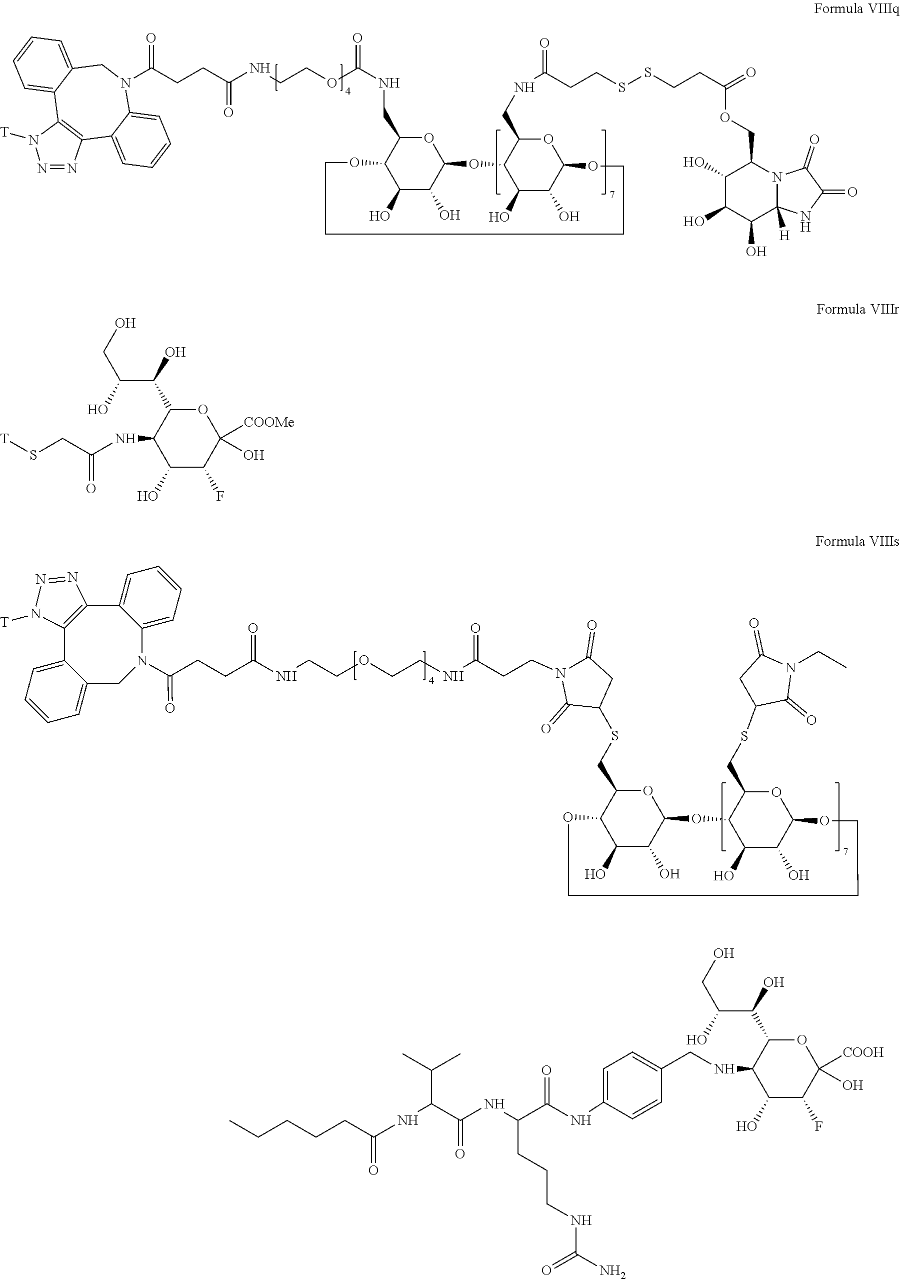

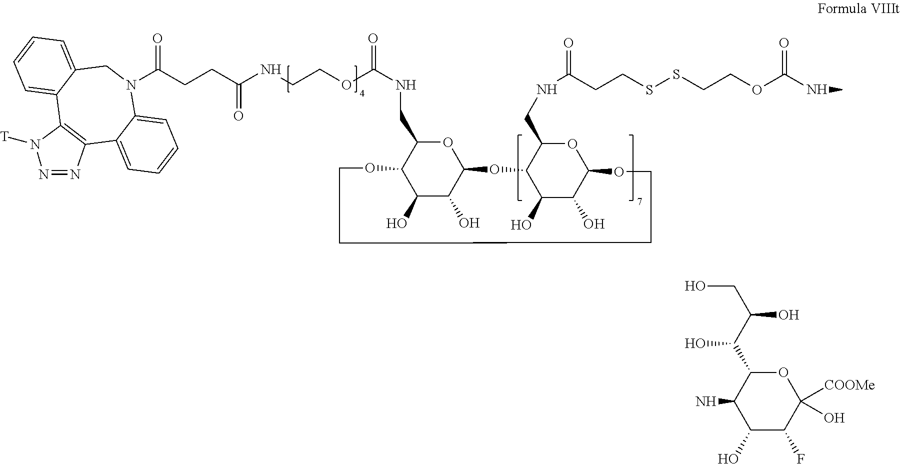

wherein T represents the targeting unit. F may be in an axial or equatorial conformation in Formulas Vb, VIb, VIIb, VIIIb, VIIIg, VIIIr, VIIIs and VIIIt.

It should also be understood that the glycosylation inhibitors described in the above formulas Va-c, VIa-b, VIIa-b or VIIIa-t may be replaced by any one of the glycosylation inhibitors described in this specification.

X) Compositions and Methods

A pharmaceutical composition comprising the conjugate according to one or more embodiments described in this specification is disclosed.

The pharmaceutical composition may further comprise one or more further components, for example a pharmaceutically acceptable carrier. Examples of suitable pharmaceutically acceptable carriers are well known in the art and may include e.g. phosphate buffered saline solutions, water, oil/water emulsions, wetting agents, and liposomes. Compositions comprising such carriers may be formulated by methods well known in the art. The pharmaceutical composition may further comprise other components such as vehicles, additives, preservatives, other pharmaceutical compositions administrated concurrently, and the like.

In an embodiment, the pharmaceutical composition comprises an effective amount of the conjugate according to one or more embodiments described in this specification.

In an embodiment, the pharmaceutical composition comprises a therapeutically effective amount of the conjugate according to one or more embodiments described in this specification.

The term “therapeutically effective amount” or “effective amount” of the conjugate may be understood as referring to the dosage regimen for achieving a therapeutic effect, for example modulating the growth of cancer cells and/or treating a patient's disease. The therapeutically effective amount may be selected in accordance with a variety of factors, including the age, weight, sex, diet and medical condition of the patient, the severity of the disease, and pharmacological considerations, such as the activity, efficacy, pharmacokinetic and toxicology profiles of the particular conjugate used. The therapeutically effective amount can also be determined by reference to standard medical texts, such as the Physicians Desk Reference 2004. The patient may be male or female, and may be an infant, child or adult.

The term “treatment” or “treat” is used in the conventional sense and means attending to, caring for and nursing a patient with the aim of combating, reducing, attenuating or alleviating an illness or health abnormality and improving the living conditions impaired by this illness, such as, for example, with a cancer disease.

In an embodiment, the pharmaceutical composition comprises a composition for e.g. oral, parenteral, transdermal, intraluminal, intraarterial, intrathecal, intra-tumoral (i.t.), and/or intranasal administration or for direct injection into tissue. Administration of the pharmaceutical composition may be effected in different ways, e.g. by intravenous, intraperitoneal, subcutaneous, intramuscular, intra-tumoral, topical or intradermal administration.

A conjugate according to one or more embodiments described in this specification or a pharmaceutical composition comprising the conjugate according to one or more embodiments described in this specification for use as a medicament is disclosed.

A conjugate according to one or more embodiments described in this specification or a pharmaceutical composition comprising the conjugate according to one or more embodiments described in this specification for use in decreasing immunosuppressive activity in a tumour is disclosed.

A conjugate according to one or more embodiments described in this specification or a pharmaceutical composition comprising the conjugate according to one or more embodiments described in this specification for use in the treatment, modulation and/or prophylaxis of the growth of tumour cells in a human or animal is also disclosed.

A conjugate according to one or more embodiments described in this specification or a pharmaceutical composition comprising the conjugate according to one or more embodiments described in this specification for use in the treatment of cancer is disclosed.

The cancer may be selected from the group of leukemia, lymphoma, breast cancer, prostate cancer, ovarian cancer, colorectal cancer, gastric cancer, squamous cancer, small-cell lung cancer, head-and-neck cancer, multidrug resistant cancer, glioma, melanoma, and testicular cancer. However, other cancers and cancer types may also be contemplated.

A method of treating, modulating and/or prophylaxis of the growth of tumour cells in a human or animal is also disclosed. The method may comprise administering the conjugate according to one or more embodiments described in this specification or the pharmaceutical composition according to one or more embodiments described in this specification to a human or animal in an effective amount.

The tumour cells may be selected from the group of leukemia cells, lymphoma cells, breast cancer cells, prostate cancer cells, ovarian cancer cells, colorectal cancer cells, gastric cancer cells, squamous cancer cells, small-cell lung cancer cells, head-and-neck cancer cells, multidrug resistant cancer cells, and testicular cancer cells.

A method for preparing the conjugate according to one or more embodiments described in this specification is disclosed. The method may comprise conjugating the glycosylation inhibitor to the targeting unit.

In the context of the method, the glycosylation inhibitor may be any glycosylation inhibitor described in this specification, for example a glycosylation inhibitor represented by formula II, III or IV.

In an embodiment of the method, the conjugate is represented by formula I, and the method comprises conjugating the glycosylation inhibitor to the linker unit; and conjugating the targeting unit to the linker unit, thus forming a conjugate represented by formula I.

In an embodiment of the method, the conjugate is represented by formula IX, and the method comprises conjugating the glycosylation inhibitor to the spacer unit; conjugating the targeting unit to the stretcher unit; and conjugating the spacer unit and the stretcher unit to each other, optionally via a specificity unit, thus forming a conjugate represented by formula IX.

In the context of the method, the targeting unit, the linker unit, the spacer unit, the stretcher unit, and or the specificity unit may be according to any one of the embodiments described in this specification, for example in any one of the sections II)-VIII).

Anything disclosed above in the context of the conjugate may also be understood as being disclosed in the context of the method(s).

The activity of the conjugates may be measured by their inhibition of cellular glycosylation by numerous methods known in the art. Glycan profiling can be done by mass spectrometry, MALDI-TOF mass spectrometry, lectin binding, lectin microarray assays, or the like, to directly measure inhibition of specific glycosylation routes by assaying decrease in the relative abundance of specific glycans compared to other glycan types, for example. Examples of suitable glycan profiling methods are described in the Examples section and further methods are well known for a person skilled in the art.

Inhibition of lectin ligand synthesis may be measured by for example using recombinant Galectins, Siglecs, or other lectins involved in immune checkpoints, and a suitable detection label. Examples of suitable lectin binding assay methods are described in the Examples section and further methods are well known for a person skilled in the art.

Inhibition of immune suppression may be measured by for example in vitro assays using target cells and immune cells, and measuring cell kill activity, cellular activation, cytokine production, or the like. Examples of suitable immune cell assay methods are well known for a person skilled in the art.

### EXAMPLES

### Example 1. Conjugation of Linker to 4-F-GlcNAc

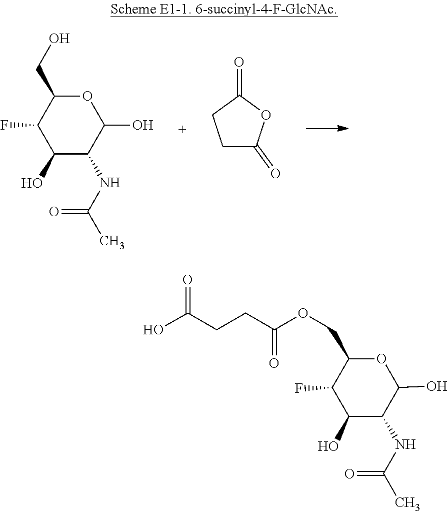 [SMILES: CC(=O)N[C@H]1C(O)O[C@H](CO)[C@@H](F)[C@@H]1O ; O=C1CCC(=O)O1 ; CC(=O)N[C@H]1C(O)O[C@H](COC(=O)CCC(=O)O)[C@@H](F)[C@@H]1O]

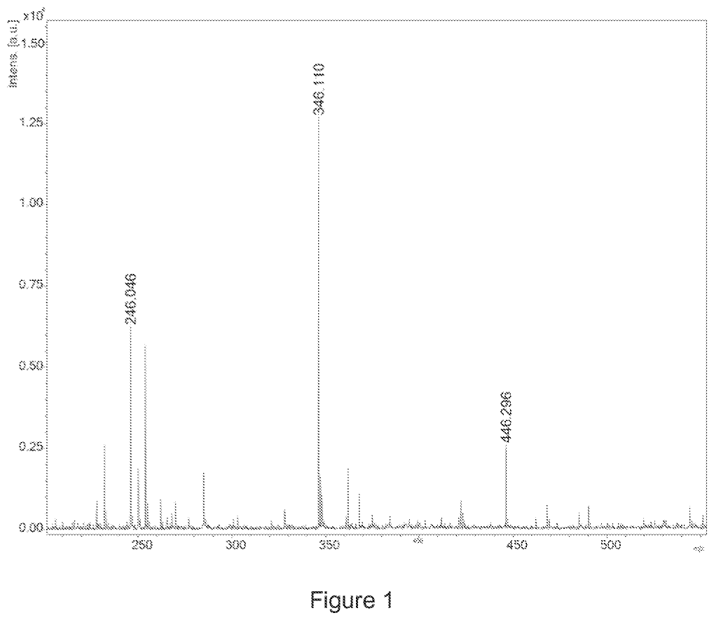

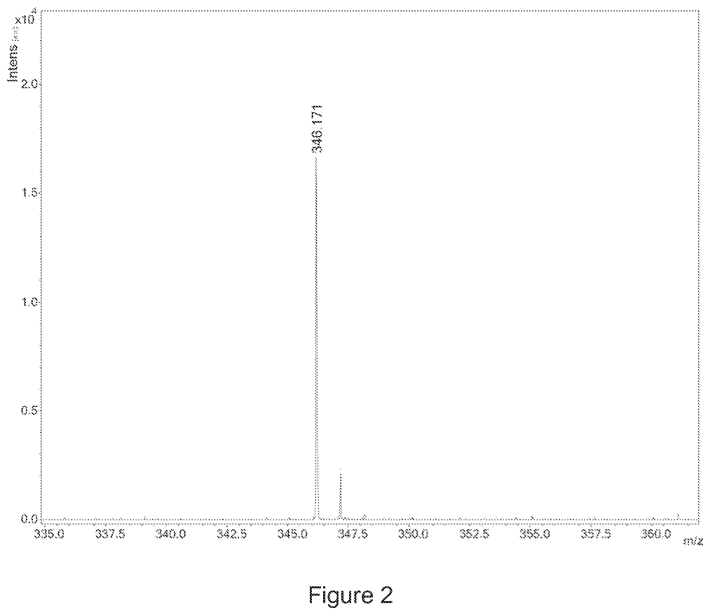

Scheme E1-1: 0.4 mg (1.8 μmol) 2-acetamido-2,4-dideoxy-4-fluoro-D-glucose (4-F-GlcNAc; Sussex Research, Ottawa, Canada), 1.5 molar excess of succinic anhydride in pyridine (2.5 μl) and 17.5 μl pyridine were stirred at room temperature (RT) for 2 hours. The crude reaction mixture was analysed by MALDI-TOF mass spectrometry (MALDI-TOF MS) with Bruker Ultraflex III TOF/TOF instrument (Bruker Daltonics, Bremen, Germany) using 2,5-dihydroxybenzoic acid (DHB) matrix, showing expected mass for 6-succinyl-4-F-GlcNAc (FIG. 1, m/z 346 [M+Na]⁺). The reaction was quenched by adding 0.5 ml ethanol. The products were purified by Äkta purifier (GE Healthcare) HPLC instrument with Sdex peptide SE column (10×300 mm, 13 μm (GE Healthcare)) in aqueous ammonium acetate buffer. 6-succinyl-4-F-GlcNAc was recovered in one of the collected fractions and detected by MALDI-TOF MS similarly as above (FIG. 2).

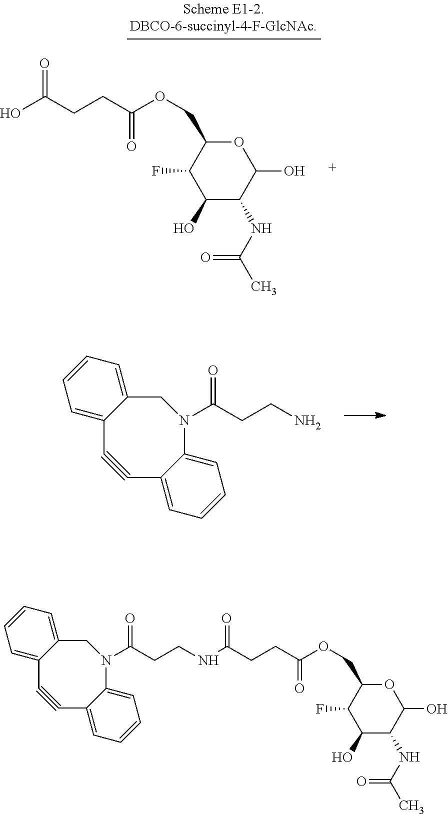 [SMILES: CC(=O)N[C@H]1C(O)O[C@H](COC(=O)CCC(=O)O)[C@@H](F)[C@@H]1O ; NCCC(=O)N1Cc2ccccc2C#Cc2ccccc21 ; CC(=O)N[C@H]1C(O)O[C@H](COC(=O)CCC(=O)NCCC(=O)N2Cc3ccccc3C#Cc3ccccc32)[C@@H](F)[C@@H]1O]

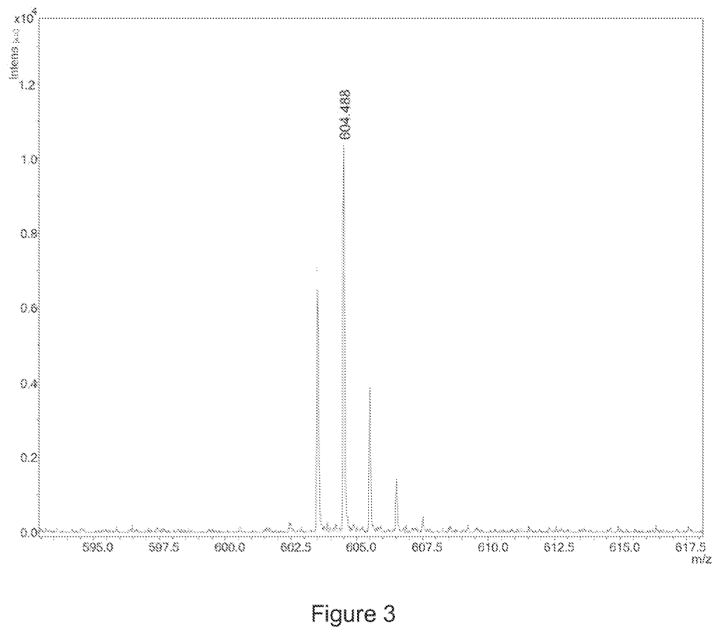

Scheme E1-2: 1 μmol 6-succinyl-4-F-GlcNAc, 10 molar excess of DBCO-amine, 5 molar excess of HBTU, 1 μl DIPEA and 108 μl DMF were stirred at RT overnight. The products were purified by Äkta purifier (GE Healthcare) HPLC instrument with Gemini 5 μm NX-C18 reverse phase column (4.6×250 mm, 110 Å (Phenomenex)) eluted with acetonitrile gradient in aqueous ammonium acetate buffer. The fractions were analysed by MALDI-TOF MS similarly as above, showing expected mass for DBCO-6-succinyl-4-F-GlcNAc (FIG. 3, m/z 604 [M+Na]⁺).

### Example 2. Conjugation of Linker-Modified 4-F-GlcNAc to Cancer-Targeting Antibody

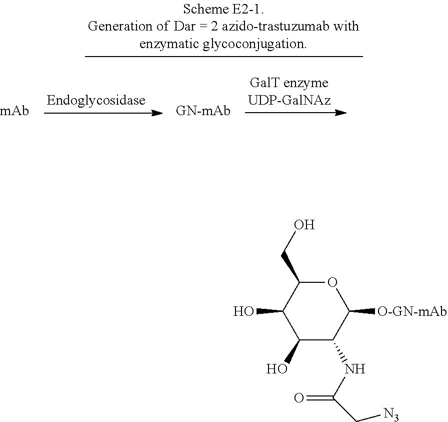

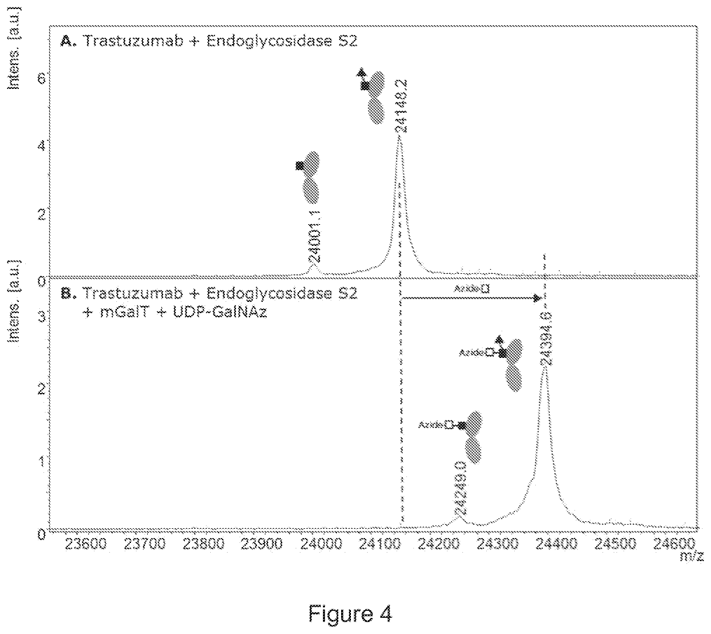

Scheme E2-1: 4 mg of anti-HER2 antibody Trastuzumab (Herceptin, Roche) was first digested with endoglycosidase S2 according to manufacturers instructions (Glycinator; Genovis, Lund, Sweden) and then incubated with 0.4 mg recombinant Y289L mutant bovine β1,4-galactosyltransferase and 1.3 mg UDP-GalNAz (both from Thermo, Eugene, USA) in the presence of Mn²⁺ containing buffer at +37° C. overnight. Azide-to-antibody ratio was determined by Fabricator enzyme digestion according to manufacturers instructions (Genovis) and MALDI-TOF MS essentially as described (Satomaa et al. 2018. Antibodies 7(2), 15). FIG. 4 shows the heavy chain Fc domains of the trastuzumab after endoglycosidase digestion (FIG. 4A; at m/z 24001 for the non-fucosylated glycoform and at m/z 24148 for the fucosylated glycoform) and then after galactosyltransferase reaction (FIG. 4B; at m/z 24249 for the non-fucosylated glycoform and at m/z 24394 for the fucosylated glycoform), with all the peaks arising from successfully azide-labeled antibody fragments, demonstrating that the azide-to-antibody ratio was 2.

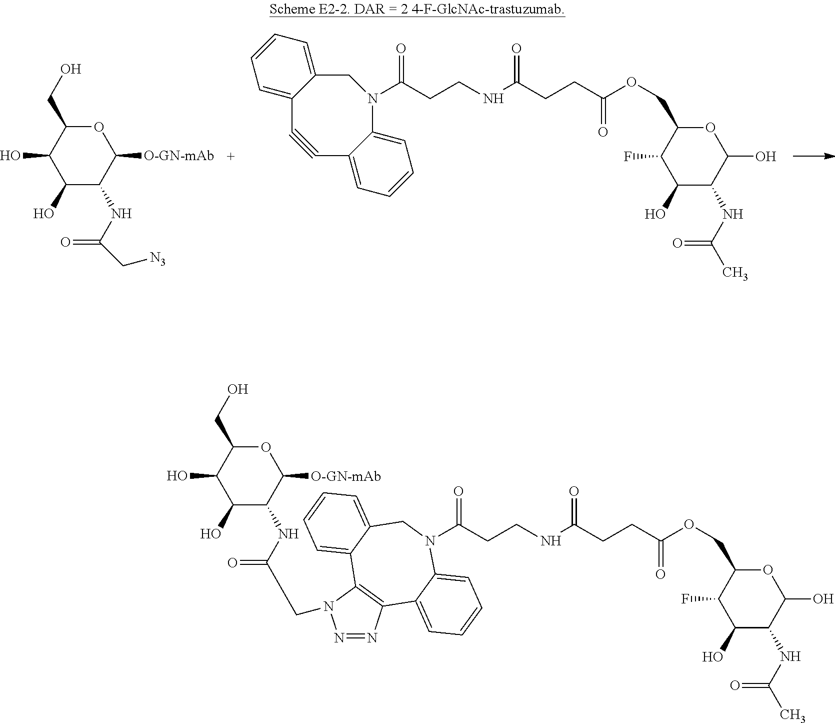

Scheme E2-2: DAR=2 azido-trastuzumab is incubated with 10 molar excess of DBCO-6-succinyl-4-F-GlcNAc in phosphate-buffered saline (PBS) at RT for 1 hour to react essentially all azide groups with the DBCO-linker compound via a triazole bond. Excess small molecules are removed by repeated filtration through Amicon centrifugal filter tubes with 10 kDa cutoff and addition of PBS. Drug-to-antibody ratio (DAR) is determined by Fabricator enzyme digestion (Genovis, Lund, Sweden) and MALDI-TOF MS essentially as described (Satomaa et al. 2018. Antibodies 7(2), 15). The product is characterized as DAR=2 4-F-GlcNAc-trastuzumab by observing that all detectable heavy chain Fc fragments have gained +604 m/z compared to non-conjugated DAR=2 azido-trastuzumab.

### Example 3. Inhibition of Glycosylation in Cancer Cells by Peracetylated 4-F-GlcNAc and Peracetylated 3-Fax-Neu5Ac

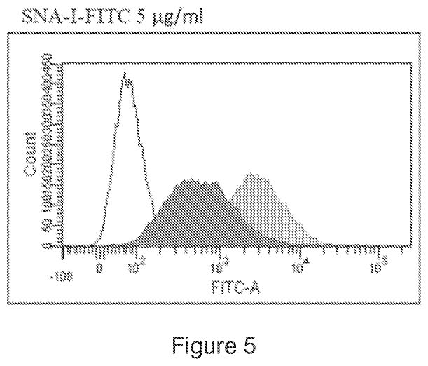

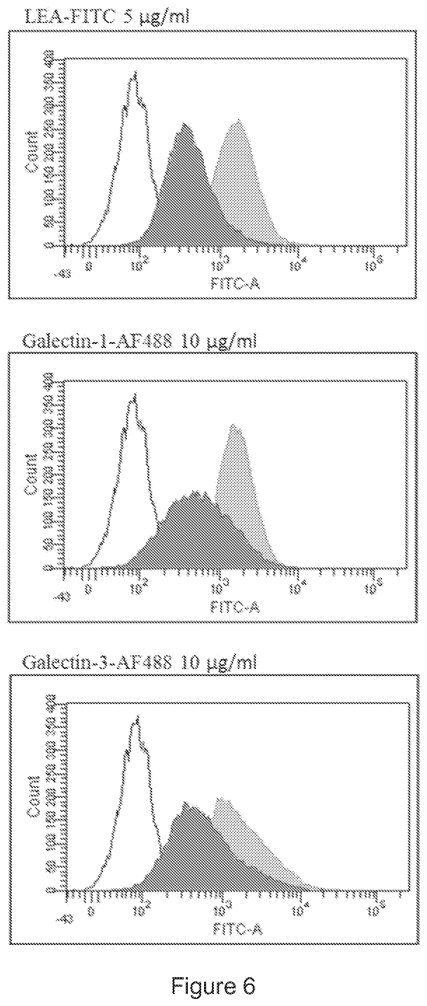

SKOV-3 ovarian carcinoma cells (ATCC, Manassas, Va., USA) were cultured according to ATCC's instructions and incubated in the presence of either 50 μM 2-acetamido-2,4-dideoxy-4-fluoro-1,3,6-tri-O-acetyl-D-glucose for 4 days (β-4-F-GlcNAc; Sussex Research, Ottawa, Canada), 100 μM 5-acetamido-3,5-dideoxy-3-fluoro-2,4,7,8,9-penta-O-acetyl-D-erythro-L-manno-2-nonulosonic acid methyl ester (β-3-Fax-Neu5Ac; Tocris Bioscience, Abingdon, United Kingdom) for 3 days, or DMSO carrier control in parallel. After the incubation, cells were stained with fluorescein-labeled lectins SNA-I-FITC for α2,6-sialylation, LEA-FITC for poly-N-acetyllactos-amines (both from EY Labs, San Mateo, Calif., USA), Alexa Fluor 488-conjugated human recombinant Galectin-1, and Alexa Fluor 488-conjugated human recombinant Galectin-3 (both from Abcam, Cambridge, United Kingdom). Cells were washed and stored on ice in the dark until analysed by FACSAriaII flow cytometer. FIG. 5 and FIG. 6 show that sialylation and Galectin ligand glycosylation were clearly decreased by the treatments.

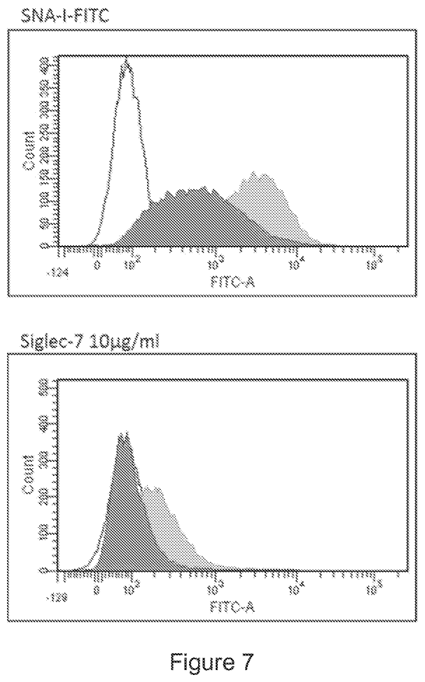

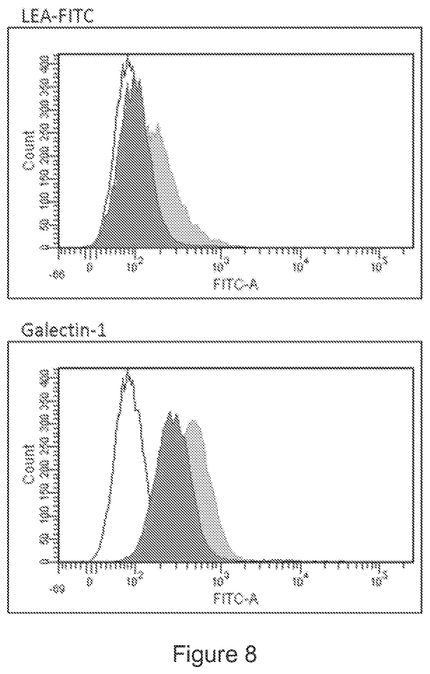

In another experiment, HSC-2 cancer cells were cultured for two days, after which glycosylation inhibitors were added to the cell culture medium: 200 μM β-3-Fax-Neu5Ac and 100 μM β-4-F-GlcNAc. The cells were then cultured for 2 days with inhibitors. In parallel, untreated cells were cultured in normal cell culture medium. For flow cytometry analysis cells were detached with trypsin, washed, and stained with FITC-conjugated lectins, AlexaFluor488-conjugated Galectin-1 and recombinant human Siglec-7 (R&D Systems) at +4° C. for 30-45 minutes (the Siglec-samples were further stained with AlexaFluor488-conjugated anti-human IgG antibody at +4° C. for 30-45 minutes). FACS was performed as above. FIG. 7 and FIG. 8 show that both sialylation/Siglec-7 ligand glycosylation and Galectin-1 ligand glycosylation were clearly decreased by the treatments.

### Example 4. Inhibition of Glycosylation in Target Cells by DAR=2 4-F-GlcNAc-Trastuzumab

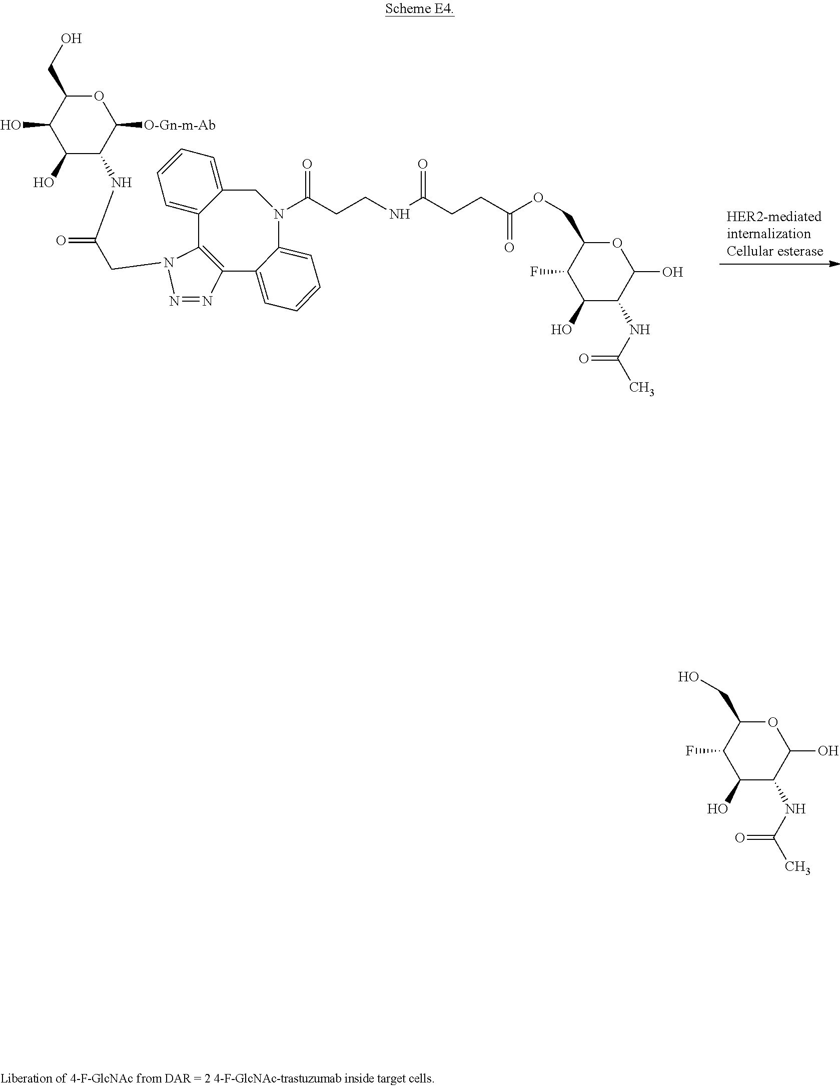

SKOV-3 ovarian carcinoma cells are cultured as described above and incubated in the presence of DAR=2 4-F-GlcNAc-trastuzumab for 3-4 days. The ADC is internalized to the cells via binding to HER2 receptors on the cell surface and the payload is released inside the cells (Scheme E4). After the incubation, cells are stained with fluorescein-labeled lectins PHA-L-FITC for complex N-glycan branching and LEA-FITC for poly-Nacetyllactos-amines (all from EY Labs, San Mateo, Calif., USA), or biotinylated human recombinant Galectin-1 and Galectin-3 (both from Abcam, Cambridge, United Kingdom), and analyzed by fluorescence-assisted cell sorting (FACS). ADC concentration is increased until detectable glycosylation inhibition is reached.

### Example 6. Maleimide-Linker and Peptide-Linker Conjugated 4-F-GlcNAc

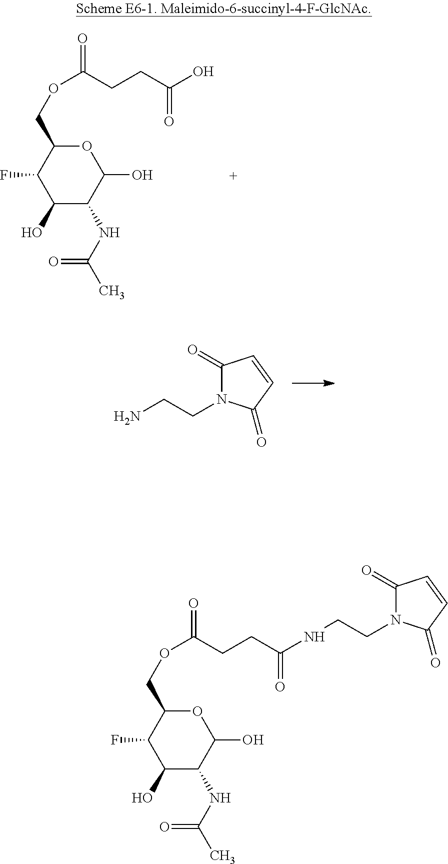 [SMILES: CC(=O)N[C@H]1C(O)O[C@H](COC(=O)CCC(=O)O)[C@@H](F)[C@@H]1O ; NCCN1C(=O)C=CC1=O ; CC(=O)N[C@H]1C(O)O[C@H](COC(=O)CCC(=O)NCCN2C(=O)C=CC2=O)[C@@H](F)[C@@H]1O]

Scheme E6-1. 6-succinyl-4-F-GlcNAc is combined with 10 molar excess of N-(2-aminoethyl)maleimide (Sigma) and 5 molar excess of HBTU in DMF with 1% DIPEA and stirred at RT overnight. The products are purified by Äkta purifier (GE Healthcare) HPLC instrument with Gemini 5 μm NX-C18 reverse phase column (4.6×250 mm, 110 Å (Phenomenex)) eluted with acetonitrile gradient in aqueous ammonium acetate buffer. The fractions are analysed by MALDI-TOF MS similarly as above, showing expected mass for Maleimido-6-succinyl-4-F-GlcNAc at m/z 468 [M+Na]⁺.

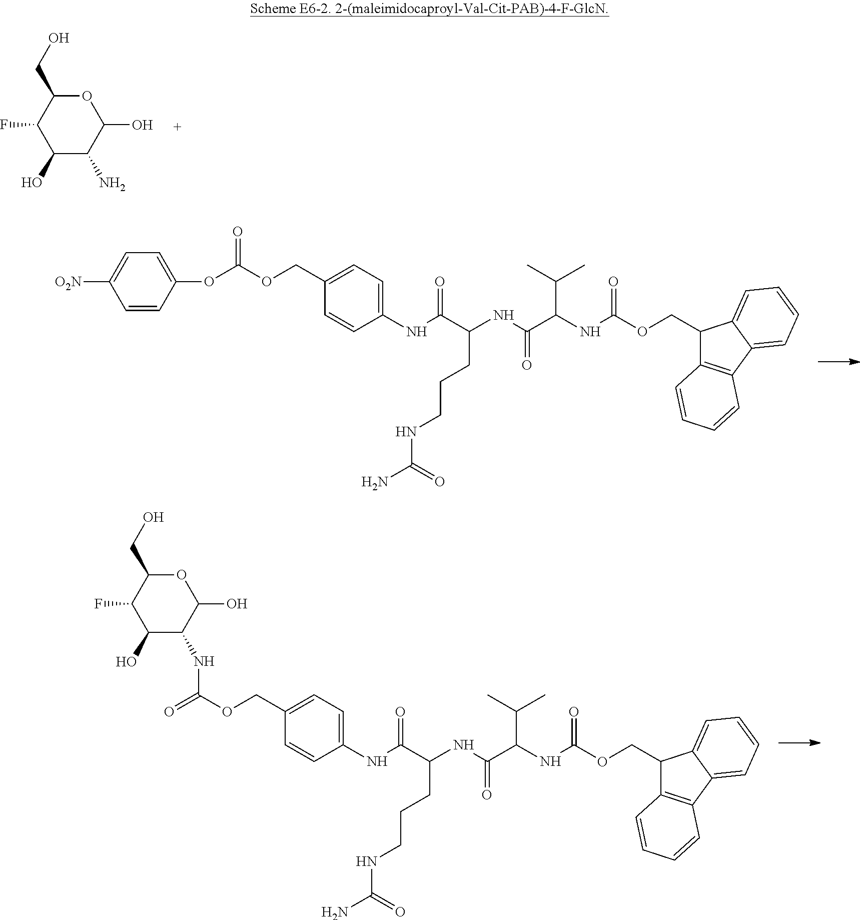 [SMILES: N[C@H]1C(O)O[C@H](CO)[C@@H](F)[C@@H]1O ; CC(C)C(NC(=O)OCC1c2ccccc2-c2ccccc21)C(=O)NC(CCCNC(N)=O)C(=O)Nc1ccc(COC(=O)Oc2ccc([N+](=O)[O-])cc2)cc1 ; CC(C)C(NC(=O)OCC1c2ccccc2-c2ccccc21)C(=O)NC(CCCNC(N)=O)C(=O)Nc1ccc(COC(=O)N[C@H]2C(O)O[C@H](CO)[C@@H](F)[C@@H]2O)cc1]

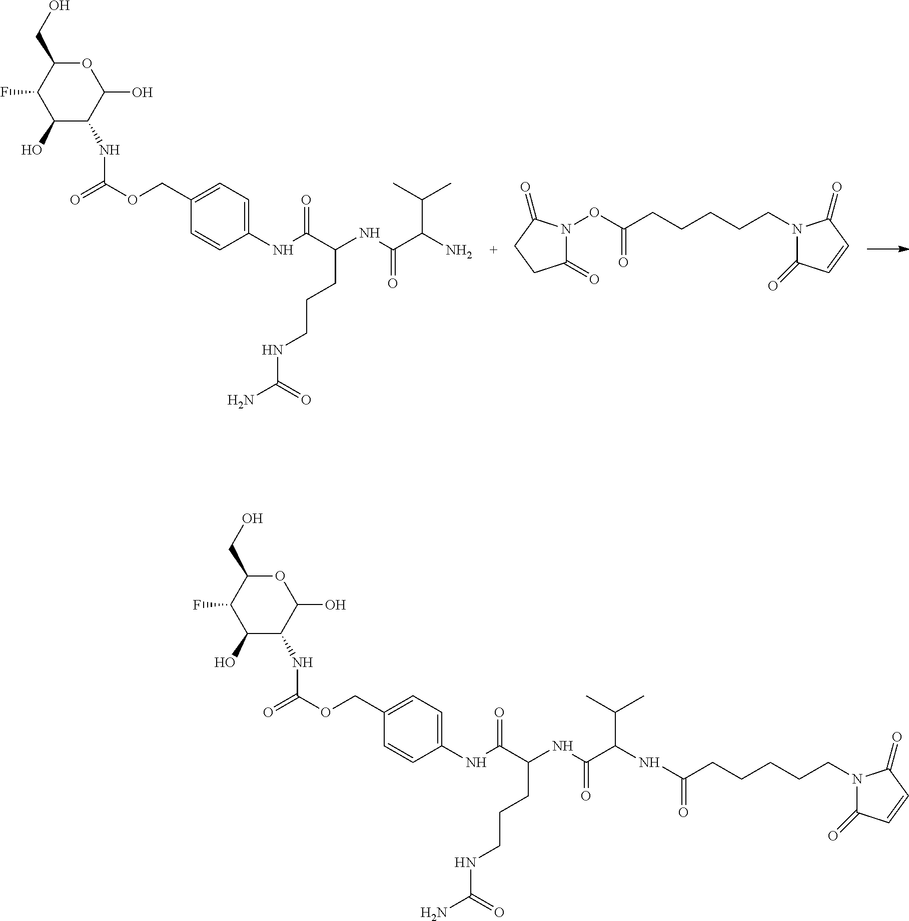 [SMILES: CC(C)C(N)C(=O)NC(CCCNC(N)=O)C(=O)Nc1ccc(COC(=O)N[C@H]2C(O)O[C@H](CO)[C@@H](F)[C@@H]2O)cc1 ; O=C(CCCCCN1C(=O)C=CC1=O)ON1C(=O)CCC1=O ; CC(C)C(NC(=O)CCCCCN1C(=O)C=CC1=O)C(=O)NC(CCCNC(N)=O)C(=O)Nc1ccc(COC(=O)N[C@H]2C(O)O[C@H](CO)[C@@H](F)[C@@H]2O)cc1]

Scheme E6-2. (4-F-GlcN) is obtained from Sussex Research Laboratories (Ottawa, Ontario, Canada). It is combined with FmocVal-Cit-PAB-paranitrophenyl, Fmoc-deprotected and reacted with maleimidocaproyl-N-hydroxysuccinimide ester as described in Satomaa et al. 2018. The products are purified by Äkta purifier (GE Healthcare) HPLC instrument with Gemini 5 μm NX-C18 reverse phase column (4.6×250 mm, 110 Å (Phenomenex)) eluted with acetonitrile gradient in aqueous ammonium acetate buffer. The fractions are analysed by MALDI-TOF MS similarly as above, showing expected mass for 2-(maleimidocaproyl-Val-Cit-PAB)-4-F-GlcN at m/z 772 [M+Na]⁺.

### Example 7. Inhibition of Tumour Cell Glycosylation and Galectin Ligand Expression in Combination with Immune Checkpoint Inhibition in Tumour-Bearing Animals by DAR=2 and DAR=8 4-F-GlcN(Ac)-trastuzumab

DAR=2 4-F-GlcNAc-trastuzumab is prepared as described above.

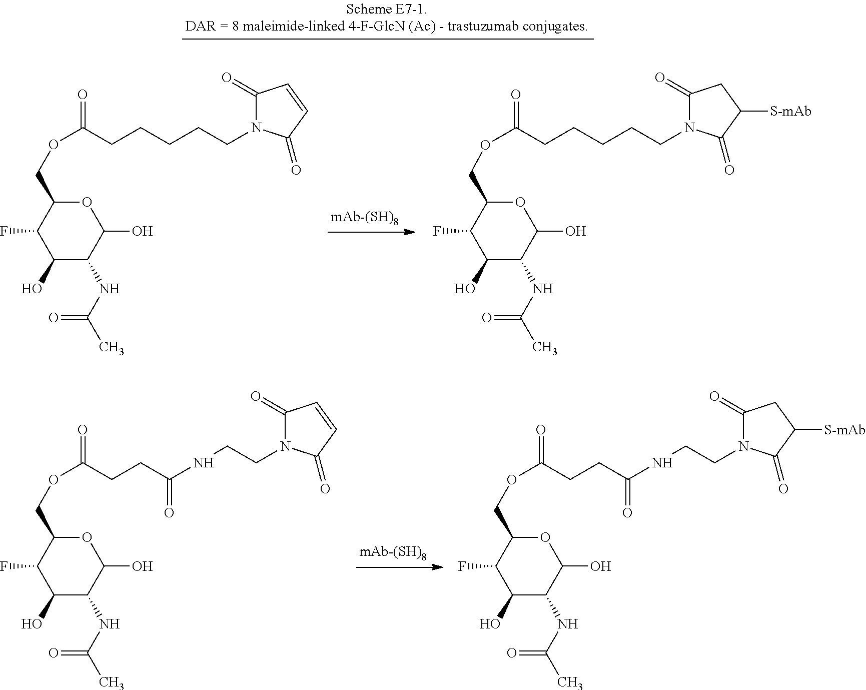

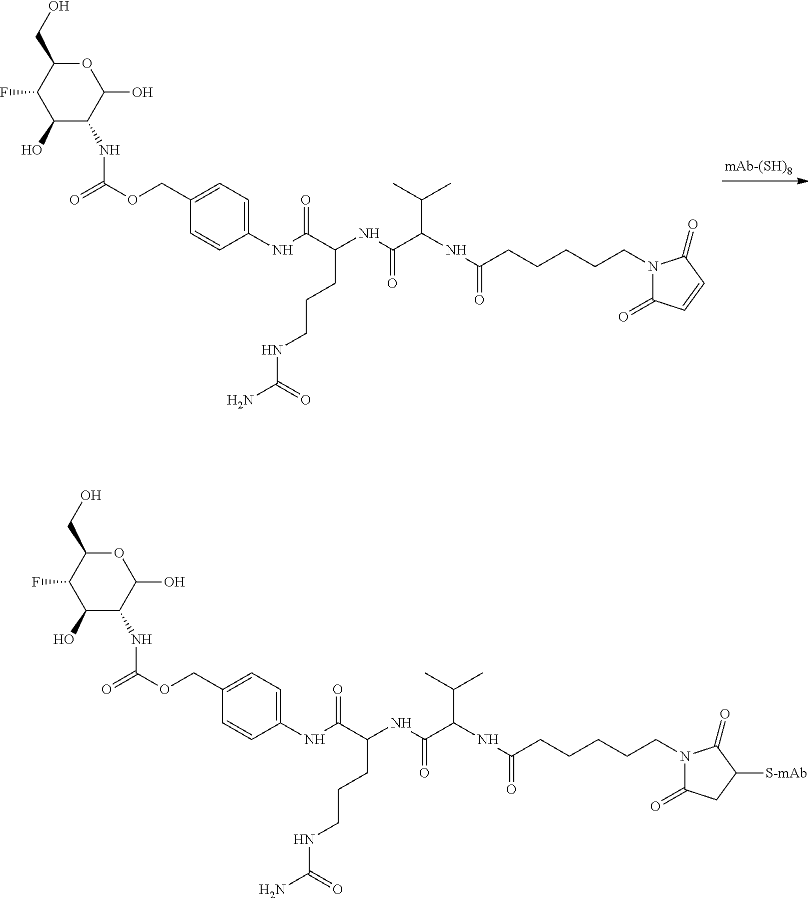

Scheme E7-1: For preparation of DAR=8 4-F-GlcN(Ac)-trastuzumab conjugates, the hinge region disulphides are reduced by TCEP as described (Satomaa et al. 2018) and combined with 8 molar excess of either 6-maleimidocaproyl-4-F-GlcNAc, maleimido-6-succinyl-4-F-GlcNAc or 2-(maleimidocaproyl-Val-Cit-PAB)-4-F-GlcN in PBS at RT for 2 hours, after which unconjugated drug-linkers are removed by repeated filtration through Amicon centrifugal filter tubes with 10 kDa cutoff and addition of PBS.

HER2-positive cancer cells are cultured as described above, injected subcutaneously to mice (about 1-10 million cells/mouse in Matrigel), and allowed to form xenograft tumors of about 100 mm³. Mice are divided into groups that receive daily 100 μl intravenous injections of either I) PBS (vehicle control), II) 10 mg/kg trastuzumab in PBS (antibody control), III) 10 mg/kg DAR=2 4-F-GlcNAc-trastuzumab in PBS, IV) 10 mg/kg DAR=8 6-maleimidocaproyl-4-F-GlcNAc-trastuzumab in PBS, V) 10 mg/kg DAR=8 6-maleimidosuccinyl-4-F-GlcNAc-trastuzumab in PBS, or VI) 10 mg/kg DAR=8 peptide-linker 4-F-GlcNAc-trastuzumab in PBS. After 5 days, about 10 mm³ pieces of tumour tissue are taken from each group and their N-glycan profiles are analyzed by MALDI-TOF MS as described (Satomaa et al. 2009. Cancer Res 69:5811-9). Smaller size of N-glycans in groups III-VI than in groups I-II, indicating lower amount of N-glycan branches and/or poly-N-acetyllactosamine chains are observed as signs of successful tumour-targeted inhibition of GlcNAc-transferases in vivo, leading to lower amounts of Galectin ligands on tumour cell surfaces, and thus less immunosuppression of antibody therapy and greater anti-cancer therapeutic activity. The ADC therapy is further combined with immune checkpoint inhibitor therapy by intravenous injection of therapeutic dose of anti-PD-1 antibody or anti-PD-L1 antibody in further groups of mice.

### Example 8. Preparation of Maleimide-Linker-Inhibitor Conjugates

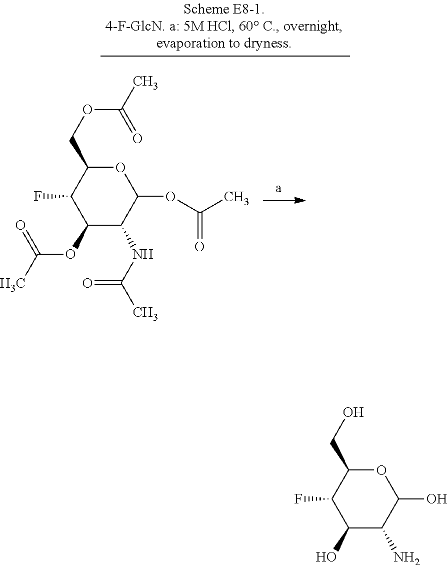 [SMILES: CC(=O)N[C@H]1C(OC(C)=O)O[C@H](COC(C)=O)[C@@H](F)[C@@H]1OC(C)=O ; N[C@H]1C(O)O[C@H](CO)[C@@H](F)[C@@H]1O]

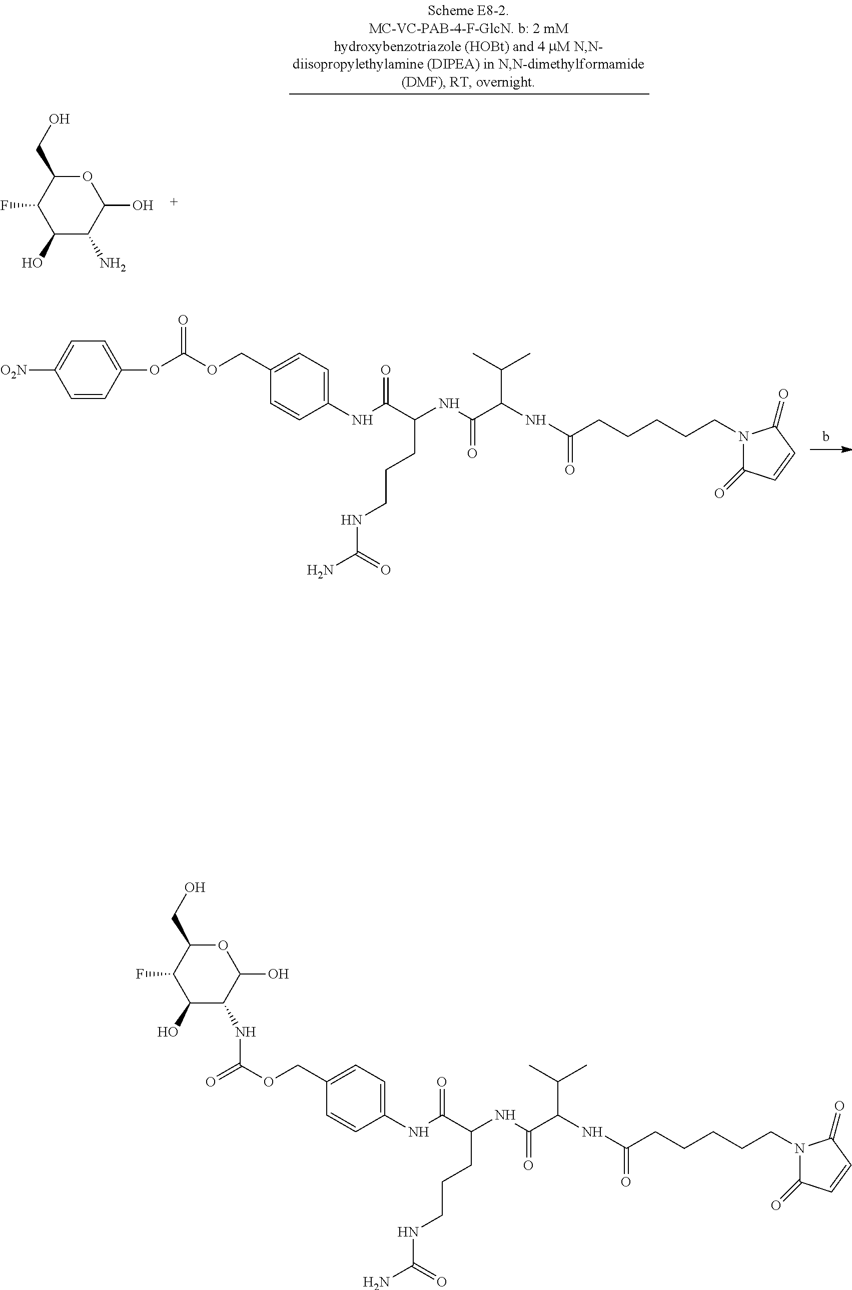 [SMILES: N[C@H]1C(O)O[C@H](CO)[C@@H](F)[C@@H]1O ; CC(C)C(NC(=O)CCCCCN1C(=O)C=CC1=O)C(=O)NC(CCCNC(N)=O)C(=O)Nc1ccc(COC(=O)Oc2ccc([N+](=O)[O-])cc2)cc1 ; CC(C)C(NC(=O)CCCCCN1C(=O)C=CC1=O)C(=O)NC(CCCNC(N)=O)C(=O)Nc1ccc(COC(=O)N[C@H]2C(O)O[C@H](CO)[C@@H](F)[C@@H]2O)cc1]

Schemes E8-1 and E8-2. β-4-F-GlcNAc (Sussex) was deacetylated (Scheme E8-1) and 2-amino-2,4-dideoxy-4-fluoro-D-glucose (4-F-GlcN) was recovered (MALDI-TOF MS: m/z 182.18, [M+H]⁺). 4-F-GlcN was combined with 2 molar equivalents (mol.eq.) of maleimidocaproyl-Val-Cit-PAB-paranitrophenyl (MC-VC-PAB-pNP, Scheme E8-2) to generate MC-VC-PAB-4-F-GlcN (MALDI-TOF MS: m/z 802.34, [M+Na]⁺). The reaction was purified with RP-HPLC as described above and the fractions containing the product were identified by MALDI-TOF MS (observed m/z 802.26 [M+Na]⁺ and 818.23 [M+K]⁺), pooled and evaporated to dryness.

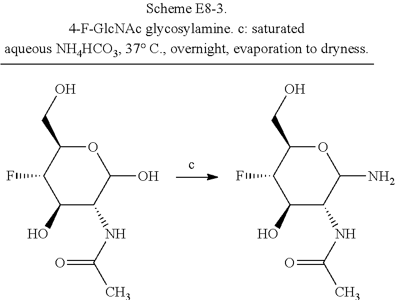

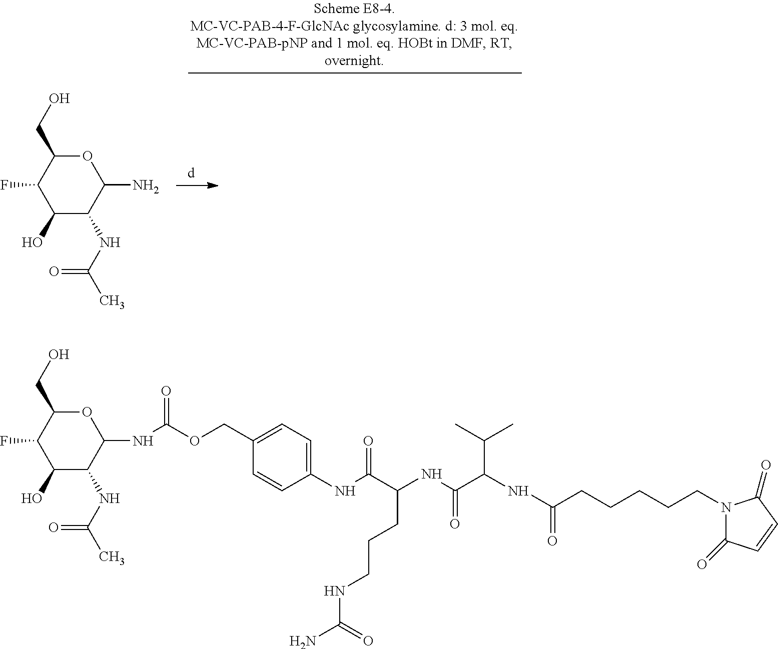 [SMILES: CC(=O)N[C@H]1C(N)O[C@H](CO)[C@@H](F)[C@@H]1O ; CC(=O)N[C@H]1C(NC(=O)OCc2ccc(NC(=O)C(CCCNC(N)=O)NC(=O)C(NC(=O)CCCCCN3C(=O)C=CC3=O)C(C)C)cc2)O[C@H](CO)[C@@H](F)[C@@H]1O]

Schemes E8-3 and E8-4. 4-F-GlcNAc (Sussex) was converted to glycosylamine (Scheme E8-3) and the resulting 4-F-GlcNAc glycosylamine was combined with MC-VC-PAB-pNP (Scheme E8-4) to generate MC-VC-PAB-4-F-GlcNAc glycosylamine (MALDI-TOF MS: m/z 843.66, [M+Na]⁺). The product was purified with RP-HPLC as described above.

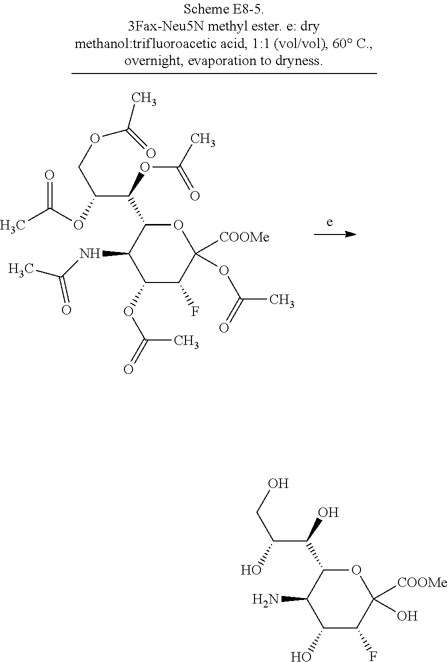 [SMILES: COC(=O)C1(OC(C)=O)O[C@@H]([C@H](OC(C)=O)[C@@H](COC(C)=O)OC(C)=O)[C@H](NC(C)=O)[C@@H](OC(C)=O)[C@H]1F ; COC(=O)C1(O)O[C@@H]([C@H](O)[C@H](O)CO)[C@H](N)[C@@H](O)[C@H]1F]

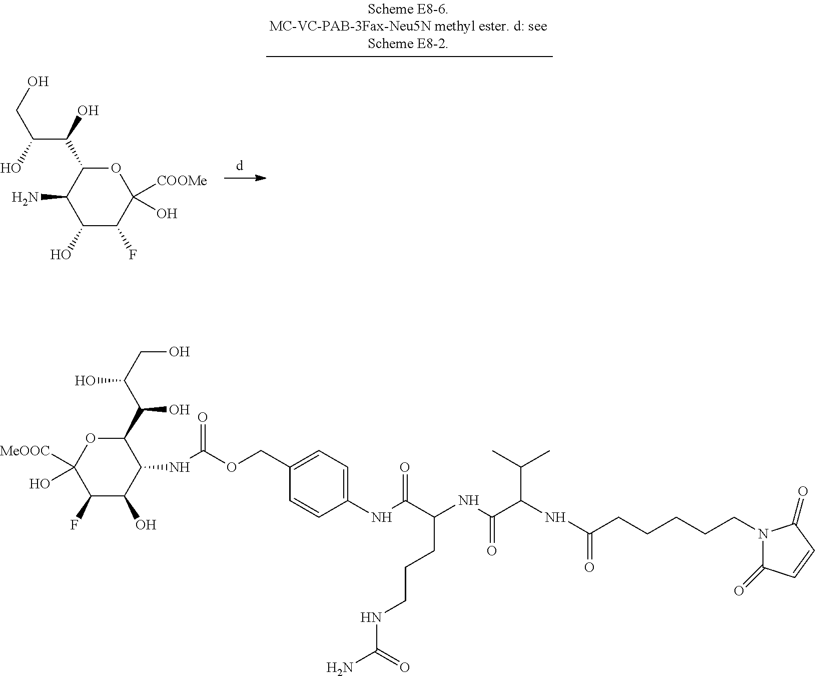 [SMILES: COC(=O)C1(O)O[C@@H]([C@H](O)[C@H](O)CO)[C@H](N)[C@@H](O)[C@H]1F ; COC(=O)C1(O)O[C@@H]([C@H](O)[C@H](O)CO)[C@H](NC(=O)OCc2ccc(NC(=O)C(CCCNC(N)=O)NC(=O)C(NC(=O)CCCCCN3C(=O)C=CC3=O)C(C)C)cc2)[C@@H](O)[C@H]1F]

Schemes E8-5 and E8-6. 4 mg β-3Fax-Neu5Ac (R&D Systems) was deacetylated (Scheme E8-3) and 3Fax-Neu5N methyl ester was recovered (MALDI-TOF MS: m/z 300.21, [M+H]⁺). The product was combined with MC-VC-PAB-pNP (Scheme E8-2) to generate MC-VC-PAB-3Fax-Neu5N methyl ester (MALDI-TOF MS: m/z 920.71 [M+Na]⁺). The product was purified with RP-HPLC as described above.

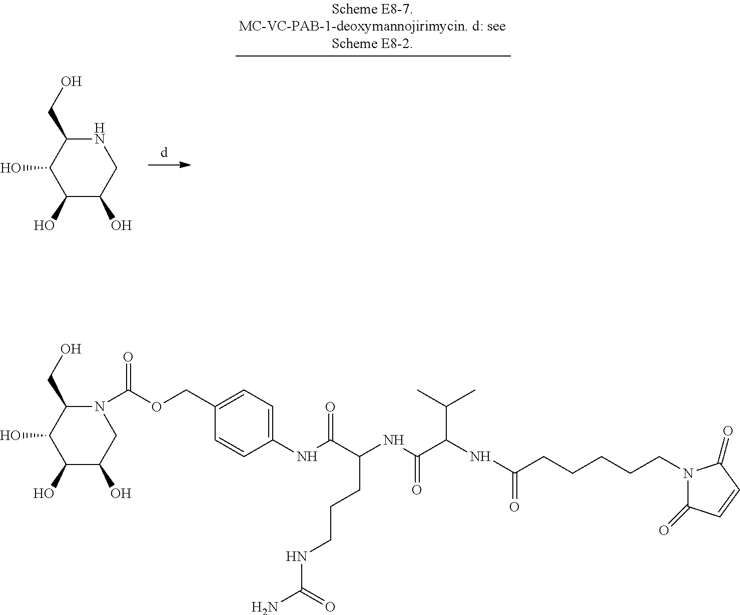 [SMILES: OC[C@H]1NC[C@@H](O)[C@@H](O)[C@@H]1O ; CC(C)C(NC(=O)CCCCCN1C(=O)C=CC1=O)C(=O)NC(CCCNC(N)=O)C(=O)Nc1ccc(COC(=O)N2C[C@@H](O)[C@@H](O)[C@H](O)[C@H]2CO)cc1]

Scheme E8-7. MC-VC-PAB-pNP was combined with 4 mol.eq. 1-deoxymannojirimycin (Carbosynth) and 4 mol.eq. HOBt in DMF to generate MC-VC-PAB-1-deoxymannojirimycin (MALDI-TOF MS: m/z 784.4, [M+Na]⁺). The product was purified with RP-HPLC as described above.

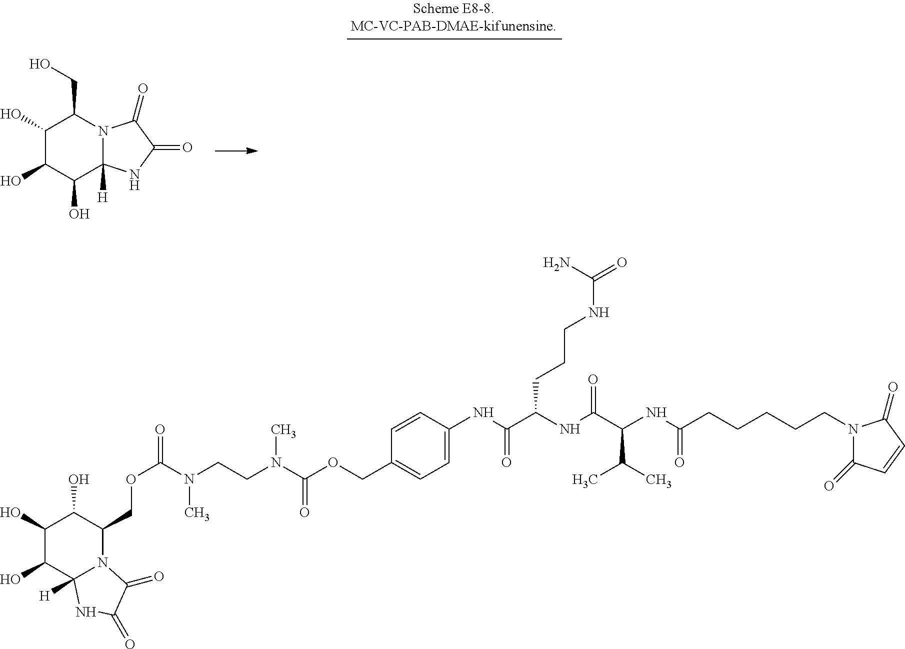 [SMILES: O=C1N[C@@H]2[C@@H](O)[C@@H](O)[C@H](O)[C@@H](CO)N2C1=O ; CC(C)[C@H](NC(=O)CCCCCN1C(=O)C=CC1=O)C(=O)N[C@@H](CCCNC(N)=O)C(=O)Nc1ccc(COC(=O)N(C)CCN(C)C(=O)OC[C@@H]2[C@@H](O)[C@H](O)[C@H](O)[C@H]3NC(=O)C(=O)N23)cc1]

Scheme E8-8. 100 mg kifunensine (Carbosynth) was reacted with MC-VC-PAB-1,2-dimethylethylenediamine (MC-VC-PAB-DMAE, Levena Biopharma) to generate 16 mg MC-VC-PAB-DMAE-kifunensine (MS: m/z 946.1, [M+H]⁺). The product was purified with RP-HPLC (data not shown).

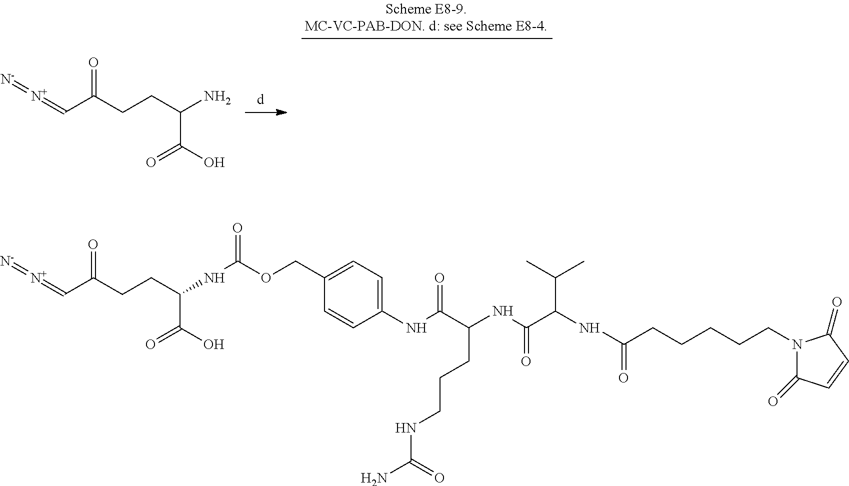 [SMILES: [N-]=[N+]=CC(=O)CCC(N)C(=O)O ; CC(C)C(NC(=O)CCCCCN1C(=O)C=CC1=O)C(=O)NC(CCCNC(N)=O)C(=O)Nc1ccc(COC(=O)N[C@@H](CCC(=O)C=[N+]=[N-])C(=O)O)cc1]

Scheme E8-9. 6-diazo-5-oxo-L-norleucine (DON, Carbosynth) was dissolved in DMSO, combined with MC-VC-PAB-pNP in DMF (DMSO:DMF=50:50, vol/vol) supplemented with HOBt, and incubated at RT for two days to generate MC-VC-PAB-DON (MALDI-TOF MS: m/z 792.56, [M+Na]⁺). The product was purified with RP-HPLC as described above.

### Example 9. Conjugation of Maleimide-Linker-Inhibitors to Cancer-Targeting Antibodies

For conjugation of maleimide-linker-inhibitors to Trastuzumab, the hinge region disulphides were reduced by tris(2-carboxyethyl)phosphine (TCEP; see Satomaa et al. 2018): 25 μM mAb was reacted with 20-40 mol.eq. TCEP in 1 mM diethylenetriaminepentaacetic acid (DTPA) in PBS at +37° C. for about 1.5 h. The reduced antibody was combined with a molar excess of maleimide-linker-inhibitor and reacted at RT for 1.5-2 hours, after which unconjugated drug-linkers were removed by repeated filtration through Amicon centrifugal filter tubes with 30 kDa cutoff and addition of PBS.

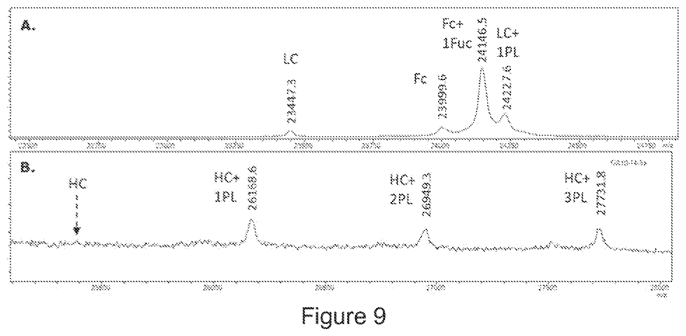

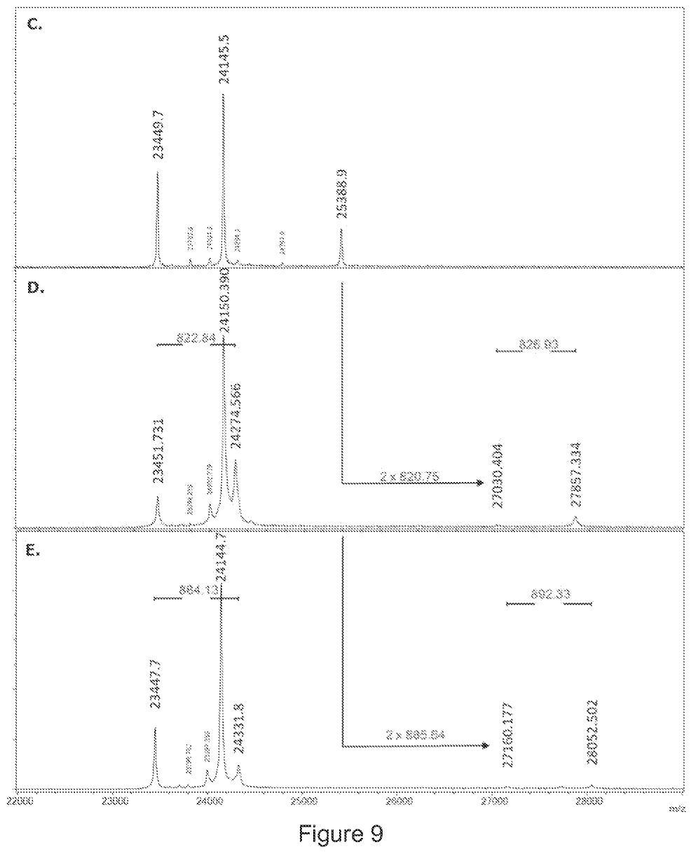

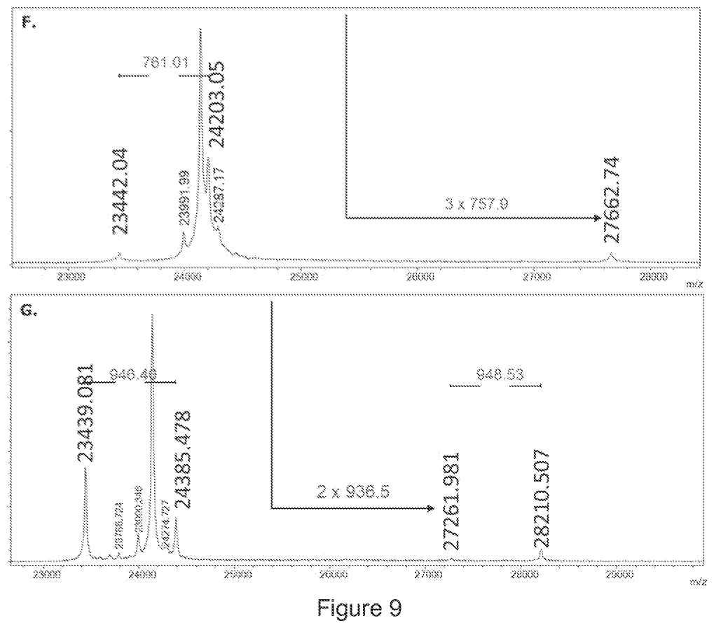

The conjugates were analyzed as by MALDI-TOF MS in dihydroxyacetophenone (DHAP) matrix as antibody fragments after Fabricator and Glycinator digestion in PBS (Genovis; according to manufacturer's instructions), denaturation with added 6 M guanidine-HCL and reduction with added 2 mM dithiothreitol (DTT) for 0.5 h at +60° C., and microscale chromatography with Poros R2 reversed phase material essentially as described (Satomaa et al. 2018). The drug-to-antibody ratio (DAR) was calculated based on relative intensities of the observed antibody fragments. FIG. 9 shows MALDI-TOF MS analysis results of trastuzumab conjugates successfully prepared with MC-VC-PAB-4-F-GlcN (FIG. 9A-B, DAR=4-8), MC-VC-PAB-4-F-GlcNAc glycosylamine (FIG. 9C-D, DAR=4-8), MC-VC-PAB-3Fax-Neu5N(FIG. 9E, DAR=4-8), MC-VC-PAB-1-deoxymannojirimycin (FIG. 9F, DAR=8) and MC-VC-PAB-DMAE-kifunensine (FIG. 9G, DAR=4-8).

### Example 10. Preparation of DBCO-Linker-Inhibitor Conjugates

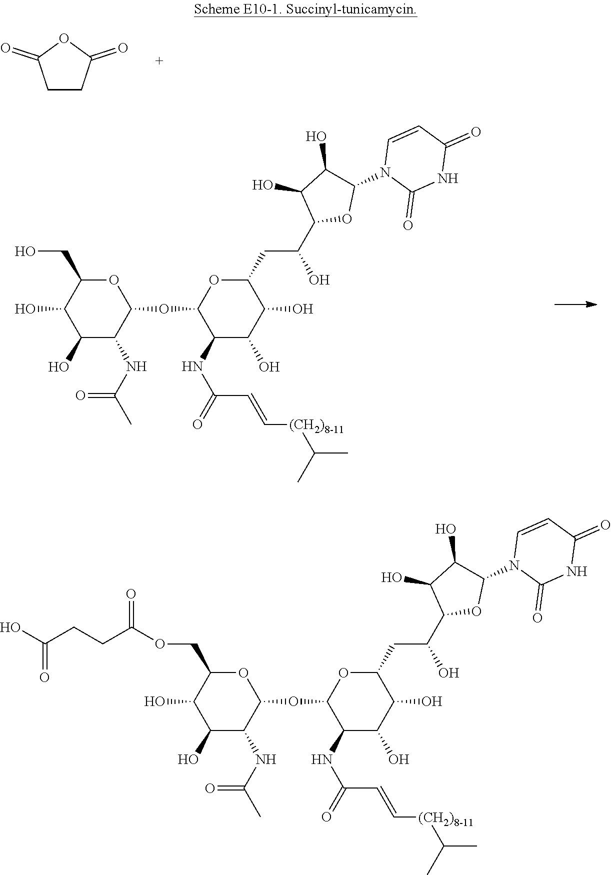 [SMILES: O=C1CCC(=O)O1 ; CC(=O)N[C@H]1[C@@H](O[C@@H]2O[C@H](C[C@@H](O)[C@H]3O[C@@H](n4ccc(=O)[nH]c4=O)[C@H](O)[C@@H]3O)[C@H](O)[C@H](O)[C@H]2NC(=O)/C=C/CC(C)C)O[C@H](CO)[C@@H](O)[C@@H]1O ; CC(=O)N[C@H]1[C@@H](O[C@@H]2O[C@H](C[C@@H](O)[C@H]3O[C@@H](n4ccc(=O)[nH]c4=O)[C@H](O)[C@@H]3O)[C@H](O)[C@H](O)[C@H]2NC(=O)/C=C/CC(C)C)O[C@H](COC(=O)CCC(=O)O)[C@@H](O)[C@@H]1O]

Scheme E10-1: Tunicamycin (Sigma) and a molar excess of succinic anhydride in pyridine were stirred at RT. The reaction mixture was analysed by MALDI-TOF MS as above, showing expected mass for succinyl-tunicamycin (a major component with C₁₄ fatty acid chain at m/z 953.63, [M+Na]⁺). The products were purified with RP-HPLC and detected in the collected fractions by MALDI-TOF MS.

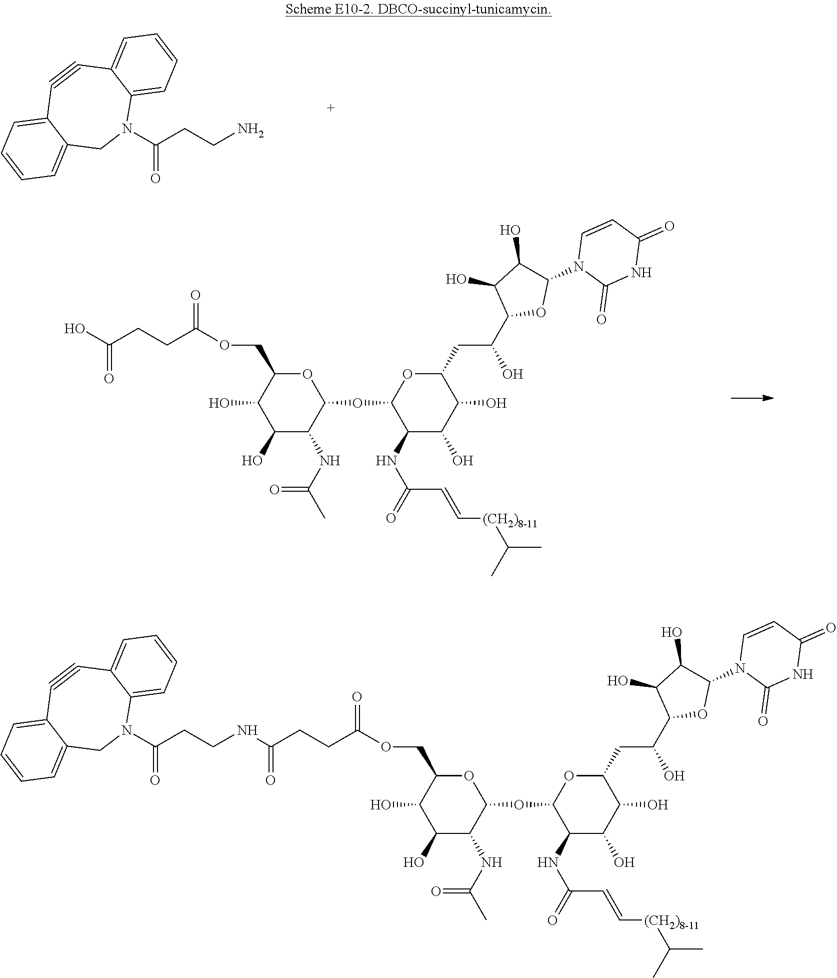 [SMILES: NCCC(=O)N1Cc2ccccc2C#Cc2ccccc21 ; CC(=O)N[C@H]1[C@@H](O[C@@H]2O[C@H](C[C@@H](O)[C@H]3O[C@@H](n4ccc(=O)[nH]c4=O)[C@H](O)[C@@H]3O)[C@H](O)[C@H](O)[C@H]2NC(=O)/C=C/CC(C)C)O[C@H](COC(=O)CCC(=O)O)[C@@H](O)[C@@H]1O ; CC(=O)N[C@H]1[C@@H](O[C@@H]2O[C@H](C[C@@H](O)[C@H]3O[C@@H](n4ccc(=O)[nH]c4=O)[C@H](O)[C@@H]3O)[C@H](O)[C@H](O)[C@H]2NC(=O)/C=C/CC(C)C)O[C@H](COC(=O)CCC(=O)NCCC(=O)N2Cc3ccccc3C#Cc3ccccc32)[C@@H](O)[C@@H]1O]

Scheme E10-2: Succinyl-tunicamycin and a molar excess of DBCO-amine were stirred at RT overnight in DMF supplemented with a molar excess of HBTU and DIPEA. The products showed expected mass for DBCO-succinyl-tunicamycin by MALDI-TOF MS (major peaks at m/z 1226.10 and 1240.12, [M+Na]⁺, for components with C₁₇ and C₁₈ fatty acid chains, respectively).

### Example 11. Acylated 1-deoxymannojirimycin and 1-deoxynojirimycin Derivatives

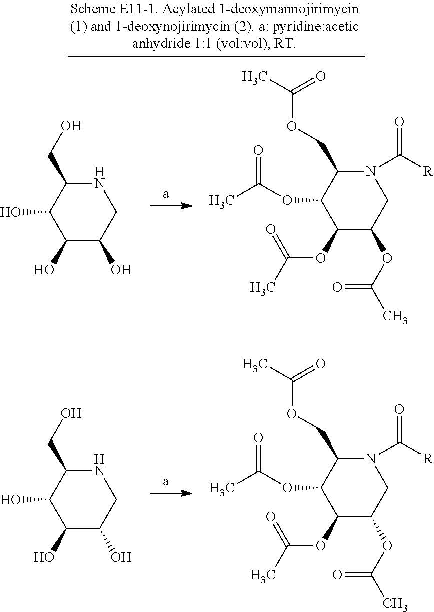

Scheme E11.1. 1-deoxymannojirimycin (Carbosynth) was peracetylated and the reaction was monitored by MALDI-TOF MS as above, showing expected mass for 5-N-acetyl-1-deoxymannojirimycin (Scheme E11.1, Compound 1, R═CH₃) at m/z 396.27 [M+Na]⁺. 1-deoxynojirimycin (Carbosynth) is reacted similarly to produce 5-N-acetyl-1-deoxynojirimycin (Scheme E11.1, Compound 2, R═CH₃). Such compounds are effective inhibitors of N-glycan processing mannosidase I and glucosidase enzymes, respectively, and thus reduce Galectin and Siglec glycan ligands, as well as other N-glycan-dependent receptor ligands, on the surface of treated cells.

### Example 12. Preparation of MC-VC-PAB-DMAE-Inhibitor Conjugates and ADCs

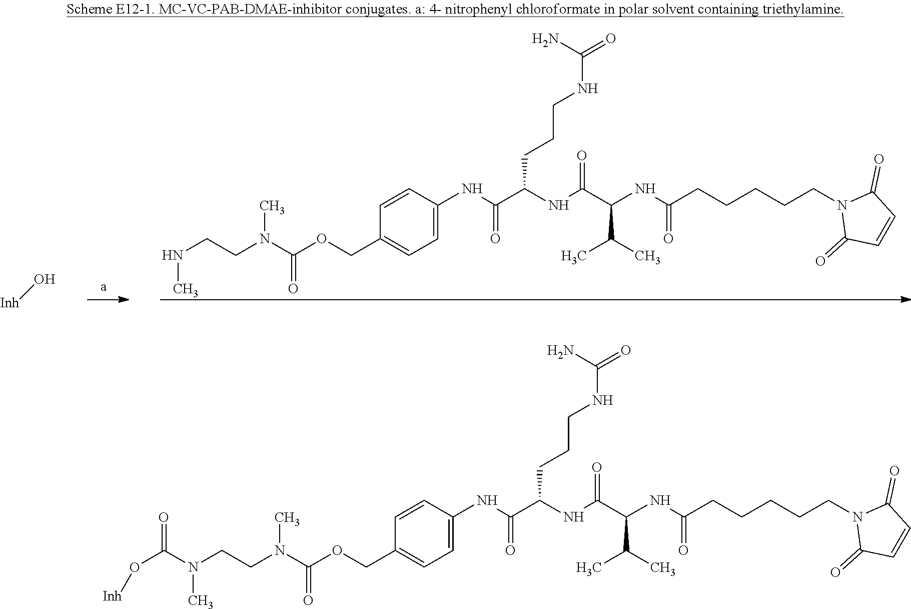

Scheme E12.1. Hydroxyl group-containing inhibitor (Inh-OH) is first reacted with 4-nitrophenyl chloroformate in tetrahydrofuran (THF; or other polar solvent based on solubility of the reactants) containing triethylamine on ice (at 0° C.) for 1.5 h. Then MC-VC-PAB-DMAE is added and the reaction is allowed to proceed at RT for 1 h. Products are detected with MALDI-TOF MS.

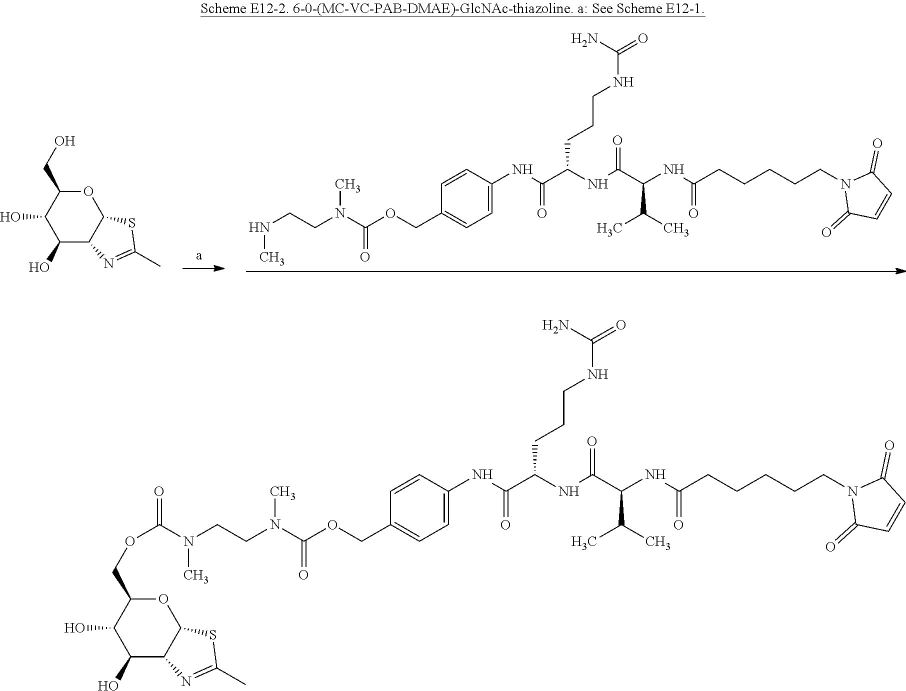 [SMILES: CC1=N[C@@H]2[C@@H](O)[C@H](O)[C@@H](CO)O[C@@H]2S1 ; CNCCN(C)C(=O)OCc1ccc(NC(=O)[C@H](CCCNC(N)=O)NC(=O)[C@@H](NC(=O)CCCCCN2C(=O)C=CC2=O)C(C)C)cc1 ; CC1=N[C@@H]2[C@@H](O)[C@H](O)[C@@H](COC(=O)N(C)CCN(C)C(=O)OCc3ccc(NC(=O)[C@H](CCCNC(N)=O)NC(=O)[C@@H](NC(=O)CCCCCN4C(=O)C=CC4=O)C(C)C)cc3)O[C@@H]2S1]

Scheme E12.1. GlcNAc-thiazoline (Carbosynth) is first reacted with 4-nitrophenyl chloroformate in tetrahydrofuran (THF; or other polar solvent based on solubility of the reactants) containing triethylamine on ice (at 0° C.) for 1.5 h. Then MC-VC-PAB-DMAE (Levena Biopharma) is added and the reaction is allowed to proceed at RT for 1 h. Products are detected with MALDI-TOF MS: m/z 407 for 6-O-(MC-VC-PAB-DMAE)-GlcNAc-thiazoline, [M+Na]⁺, and m/z 955 for 6-O-(MC-VC-PAB-DMAE)-GlcNAc-thiazoline, [M+Na]⁺.

### Example 14. Inhibition of Glycosylation in Target Cells by Glycosylation Inhibitor-ADCs

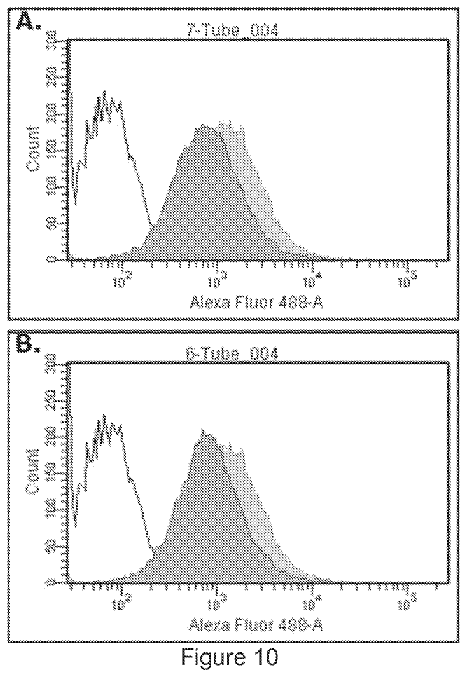

SKBR-3 breast cancer cells (ATCC) were cultured in recommended conditions and incubated with glycosylation inhibitors and ADCs as described above. The cells were then subjected to labeling with SNA-I lectin and FACS analysis as described above. As shown in FIG. 10, both cells incubated for three days with 500 nM trastuzumab-MC-VC-PAB-3Fax-Neu5N, DAR=4-8 (FIG. 10A) and cells incubated for four days with 10 nM Trastuzumab-MC-VC-PAB-DMAE-kifunensine, DAR=4-8 (FIG. 10B) had reduced staining with SNA-I lectin. This demonstrated that the ADCs had inhibited cell surface sialylation in the cells, and in the case of the kifunensine-ADC, inhibited N-glycosylation-associated cell surface sialylation.

SKBR-3 cells treated for four days with kifunensine-ADCs, both with 10 nM and 1 μM trastuzumab-MC-VC-PAB-DMAE-kifunensine, DAR=4-8, as well as with 10 μM kifunensine, were also subjected to N-glycan profiling with MALDI-TOF MS essentially as described in Leijon et al. 2017, J Clin Endocrinol Metab 102(11):3990-4000, although without the deparaffinization step. The N-glycan profiles comprising the cellular neutral N-glycans showed increased number of hexose residues in the high-mannose type N-glycan signals with assigned monosaccharide compositions Man₅₋₉GlcNAc₂ (m/z 1257, m/z 1419, m/z 1581, m/z 1743 and m/z 1905 for [M+Na]⁺ adduct ions, respectively; which could be relatively quantitated based on relative signal intensity as described in Leijon et al. 2017; data not shown) when the cells were subjected to either kifunensine or kifunensine-ADC treatment. In control cells (no treatment) as well as in cells treated with 1 μM trastuzumab for 3 days, the average number of mannose residues (Man) in the Man₅₋₉GlcNAc₂ glycan signal series was 7.07 and 6.96, respectively, whereas in cells treated in parallel with kifunensine, 10 nM or 1 μM trastuzumab-MC-VC-PAB-DMAE-kifunensine, DAR=4-8, the average number of mannose residues (Man) in the Man₅₋₉GlcNAc₂ glycan signal series was increased to 8.56, 7.19 and 7.23, respectively. This demonstrated effective inhibition of mannosidase I activity in both inhibitor and inhibitor-ADC treated cells.

SKBR-3 cells treated for four days with sialylation inhibitor-ADC (0.5 μM trastuzumab-MC-VC-PAB-3ax-fluoro-NeuN, DAR=4-8) were also subjected to N-glycan profiling with MALDI-TOF MS as described above, with sialylated N-glycans analyzed together with neutral N-glycans after esterification of the sialic acids essentially as described by Reiding et al. 2014, Anal Chem 86(12):5784-93. The N-glycan profiles comprising both the cellular neutral and esterified/sialylated N-glycans showed decreased relative amount of sialylated glycans when the cells were subjected to the ADC treatment: in control cells (no treatment) the proportion of sialylated glycans of the total detected glycans was 11.0%, whereas in cells treated in parallel with 0.5 μM trastuzumab-MC-VC-PAB-3ax-fluoro-NeuN, DAR=4-8, the proportion of sialylated glycans of the total detected glycans was 7.9%. This demonstrated effective inhibition of sialylation in the inhibitor-ADC treated cells.

### Example 15. ADCC Assay

SKBR-3 and SKOV-3 cells were cultured on 96-well plates in recommended conditions and incubated with or without glycosylation inhibitors or ADCs for four days as described above. Then either 1 μg/ml trastuzumab, 1 μg/ml omalizumab (Xolair; Roche) or no antibody, as well as effector NK (CD56+) cells, CD4+ cells and CD8+ cells (in combination) isolated with magnetic anti-CD56, anti-CD4 and anti-CD8 affinity beads (Miltenyi Biotec, Bergisch Gladbach, Germany) from human peripheral blood buffy coats (Finnish Red Cross Blood Service, Helsinki, Finland) or no effector cells were introduced to perform antibody-dependent cellular cytotoxicity (ADCC) assays. After 3.5 h at +37° C., cytotoxicity was assessed with commercial lactate dehydrogenase assay kit (Cytotoxicity detection kit (LDH), Thermo Fischer Scientific) and the cytotoxicities were calculated as proportion of killed cells (%, average of three parallel wells).

In an ADCC assay with SKBR-3 cells, both kifunensine and tunicamycin increased cytotoxicity % when both trastuzumab and effector cells were applied: without inhibitors cytotoxicity was on average 13.2%, with 10 μM kifunensine cytotoxicity was on average 18.5% and with 1 μM tunicamycin cytotoxicity was on average 40.4%; whereas no cytotoxicity was detected when only the inhibitors and trastuzumab were applied to the cells.

In another ADCC assay with SKBR-3 cells, both kifunensine, tunicamycin and peracetylated 4-fluoro-GlcNAc increased cytotoxicity % when both trastuzumab and effector cells were applied: without inhibitors cytotoxicity was on average about 12%, with 50 μM kifunensine cytotoxicity was on average about 19%, with 0.5 μM tunicamycin cytotoxicity was on average about 46%, and with 50 μM peracetylated 4-fluoro-GlcNAc cytotoxicity was on average about 16%; whereas the cytotoxicities when only the inhibitors and trastuzumab were applied to the cells were as follows: with 50 μM kifunensine cytotoxicity was on average about 2-3%, with 0.5 μM tunicamycin cytotoxicity was on average about 4%, and with 50 μM peracetylated 4-fluoro-GlcNAc cytotoxicity was on average about 1-2%; and without both inhibitor and effector cells no cytotoxicity was observed.

In a third ADCC assay with SKBR-3 cells, peracetylated 3ax-fluoro-Neu5Ac increased cytotoxicity % when both trastuzumab and effector cells were applied: without the inhibitor the absorbance reading in the cytotoxicity assay was on average below 0.6 and with 50 μM peracetylated 3ax-fluoro-Neu5Ac the absorbance reading in the cytotoxicity assay was on average about 0.7.

In an ADCC assay with SKOV-3 cells, both kifunensine, tunicamycin and peracetylated 4-fluoro-GlcNAc increased cytotoxicity % when both trastuzumab and effector cells were applied: without inhibitors cytotoxicity was on average about 1%, with 50 μM kifunensine cytotoxicity was on average about 2%, with 0.5 μM tunicamycin cytotoxicity was on average about 5%, and with 50 μM peracetylated 4-fluoro-GlcNAc cytotoxicity was on average about 5%; whereas the cytotoxicities when only the inhibitors and trastuzumab were applied to the cells were as follows: with both 50 μM kifunensine and 50 μM peracetylated 4-fluoro-GlcNAc no cytotoxicity was observed and with 0.5 μM tunicamycin cytotoxicity was on average about 2%; and without both inhibitor and effector cells no cytotoxicity was observed.

In conclusion, it was demonstrated that both N-glycosylation inhibition (tunicamycin), N-glycan trimming inhibition (kifunensine), GlcNAc-transferase inhibition (peracetylated 4-fluoro-GlcNAc) and sialylation inhibition (peracetylated 3ax-fluoro-Neu5Ac) act synergistically with NK/CD4+/CD8+ effector cells to increase ADCC.

### Example 16. Preparation of Inhibitor Derivatives

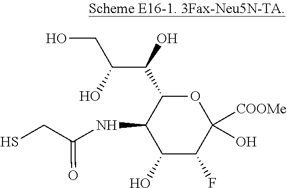 [SMILES: COC(=O)C1(O)O[C@@H]([C@H](O)[C@H](O)CO)[C@H](NC(=O)CS)[C@@H](O)[C@H]1F]

Scheme E16-1. 3Fax-Neu5N was obtained as described above and amidated with N-succinimidyl S-acetylthioacetate (Thermo Scientific Pierce SATA, Catalog number: 26102) in DMF with DIPEA, yielding a product with correct m/z of 438.25 [M+Na]⁺ in MALDI-TOF MS. The product is purified with RP-HPLC as described above and hydrolyzed with aqueous hydroxylamine according to the manufacturer's instructions to yield 3Fax-Neu5N-TA with free thiol group.

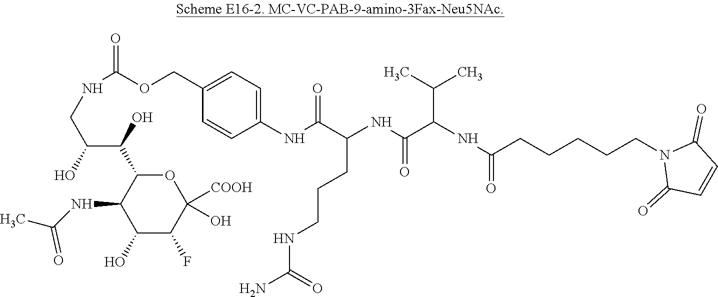 [SMILES: CC(=O)N[C@@H]1[C@@H](O)[C@@H](F)C(O)(C(=O)O)O[C@H]1[C@H](O)[C@H](O)CNC(=O)OCc1ccc(NC(=O)C(CCCNC(N)=O)NC(=O)C(NC(=O)CCCCCN2C(=O)C=CC2=O)C(C)C)cc1]

Scheme E16-2. 9-amino-3Fax-Neu5NAc was obtained from Carbosynth and it was amidated to MC-VC-PAB-pNP as described above to yield the correct product with m/z of 947.33 [M+Na]⁺ in MALDI-TOF MS. The product was purified with RP-HPLC as described above.

Several kifunensine derivatives were prepared (Schemes E16-3 and E16-4).

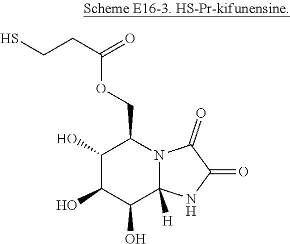 [SMILES: O=C(CCS)OC[C@@H]1[C@@H](O)[C@H](O)[C@H](O)[C@H]2NC(=O)C(=O)N12]

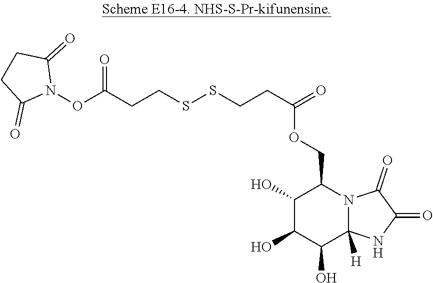 [SMILES: O=C(CCSSCCC(=O)ON1C(=O)CCC1=O)OC[C@@H]1[C@@H](O)[C@H](O)[C@H](O)[C@H]2NC(=O)C(=O)N12]

### Example 17. Preparation of Glycosylation Inhibitor ADCs

MC-VC-PAB-9-amino-3Fax-Neu5NAc was conjugated to reduced trastuzumab as described above to yield a DAR=8 ADC as shown by Fabricator digestion and MALDI-TOF MS analysis of isolated antibody fragments as described above.

MC-VC-PAB-DMAE-tunicamycin V, MC-VC-PAB-DMAE-tunicamycin VII and MC-VC-PAB-DMAE-tunicamycin X were separately conjugated to reduced trastuzumab as described above to yield DAR=8 ADCs as shown by Fabricator digestion and MALDI-TOF MS analysis of isolated antibody fragments as described above. The DAR=8 tunicamycin V ADC was shown to have retention time between DAR=3 and DAR=4 trastuzumab-MC-VC-PAB-MMAE ADCs by HIC-HPLC performed as previously described (Satomaa et al. 2018), indicating that the ADCs had similar hydrophilicity/hydrophobicity properties. The DAR=8 tunicamycin VII and X ADCs had closely similar, but longer HIC retention time.

### Example 18. Specific Inhibition of Cellular Glycosylation and Viability with Tunicamycin-ADCs

DAR=8 conjugates of MC-VC-PAB-DMAE-tunicamycin V were prepared from both trastuzumab and omalizumab (negative control antibody Xolair, Novartis). Conjugation level was shown to be DAR=8 by Fabricator digestion and MALDI-TOF MS analysis of isolated antibody fragments as described above.

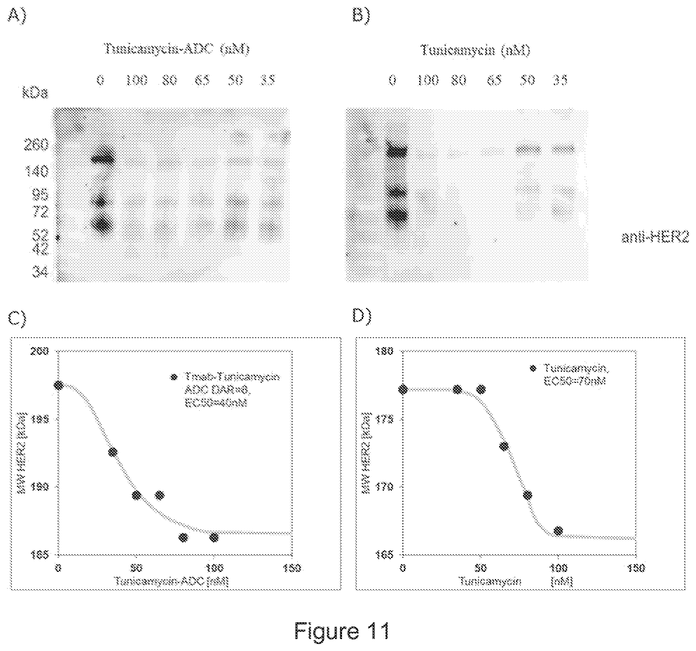

First, effect of increasing levels of tunicamycin and trastuzumab-MC-VC-PAB-DMAE-tunicamycin DAR=8 ADC on glycoprotein glycosylation were compared in SKBR-3 cells. After six days' culture, the cells were lysed and samples from each treatment were subjected to SDS-PAGE and immunoblotting with anti-HER2 antibody (anti-human ErbB2/Her2 goat polyclonal antibody AF1129, R&D Systems) with standard procedures. The results are shown in FIG. 11A-B, demonstrating that the relative MW of HER2 was decreased about 15 kDa upon inhibition of N-glycosylation. FIG. 11C-D shows analysis of the corresponding EC50 values based on the immunoblotting results, demonstrating effective inhibition of N-glycosylation with both the ADC and free tunicamycin, while the ADC had 1.75-fold lower EC50 (40 nM compared to 70 nM, respectively).

Second, effect of increasing levels of tunicamycin, tunicamycin-ADCs and trastuzumab on cellular viability were compared in SKBR-3 cells. In a first experiment, tunicamycin and trastuzumab-MC-VC-PAB-DMAE-tunicamycin DAR=8 ADC were compared in culture of SKBR-3 cells for six days. Tunicamycin had IC50 of 300 nM and the ADC had IC50 of 150 nM (data not shown) showing that the ADC had two-fold lower IC50. Further, this experiment demonstrated that the glycosylation inhibition effect of both tunicamycin and tunicamycin-ADC occurs at lower concentration than the viability inhibition effect, i.e. EC50<IC50.

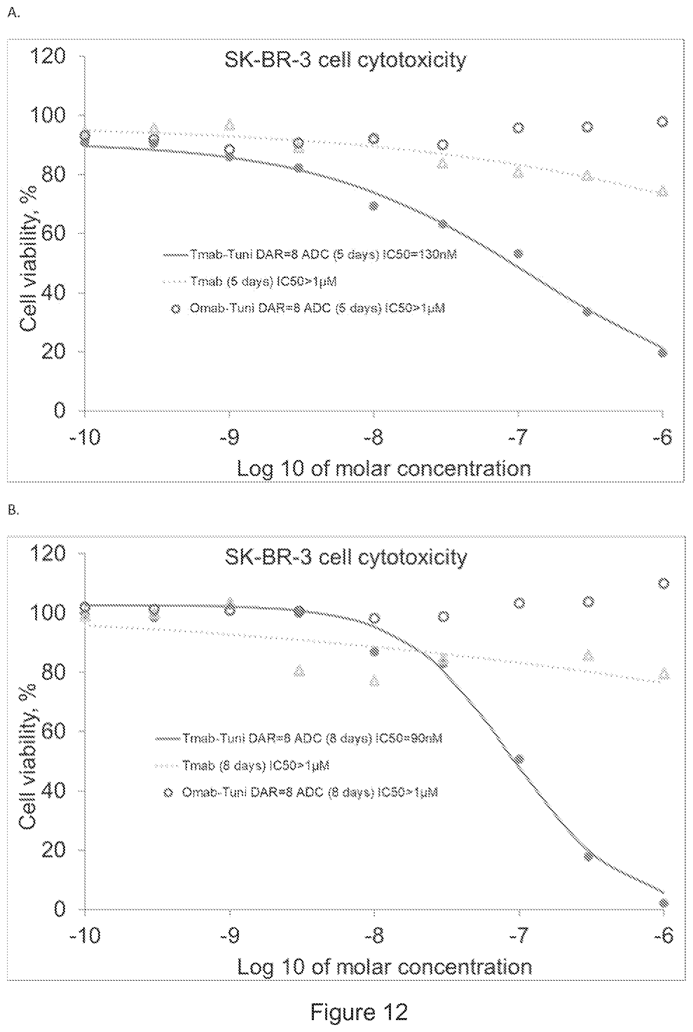

Third, effect of increasing levels of trastuzumab, trastuzumab-MC-VC-PAB-DMAE-tunicamycin DAR=8 ADC and omalizumab-MC-VC-PAB-DMAE-tunicamycin DAR=8 ADC, were compared in culture of SKBR-3 cells for either five (FIG. 12A) or eight days (FIG. 12B). The trastuzumab-ADC had IC50 of 130 nM at five days and 90 nM at eight days. Trastuzumab had only modest cytotoxicity and the IC50 was not reached at maximum concentration of 1 μM, showing that the effect of the ADC was specific. The omalizumab-ADC showed no apparent toxicity to the cells, showing that the effect of the ADC was specific and that the payload was not released during the incubation.

### Example 19. High-DAR Glycosylation Inhibitor Conjugates

Several conjugates are prepared (Schemes E19-1 to E19-5).

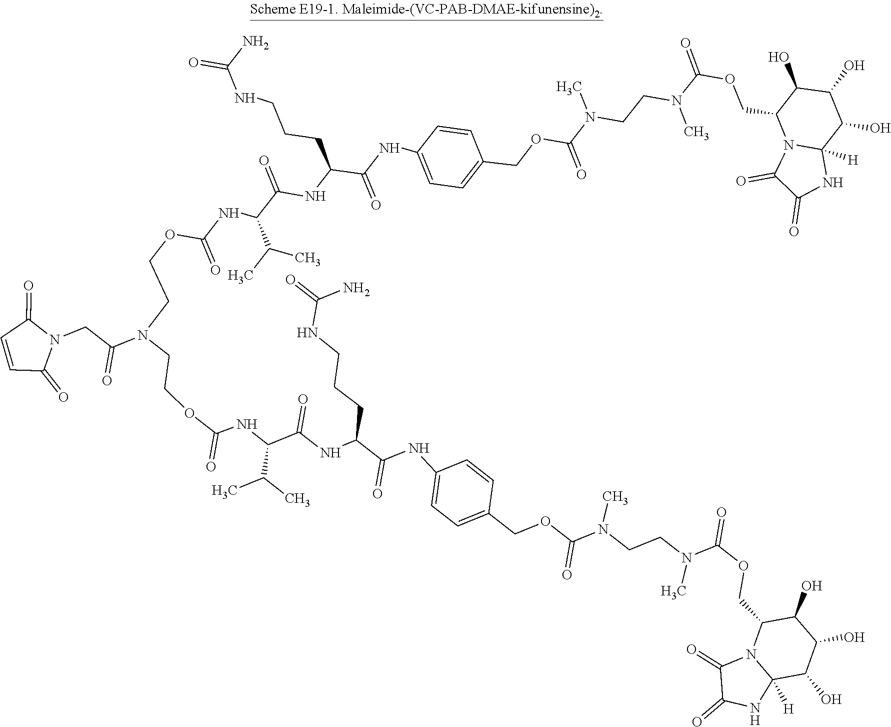 [SMILES: CC(C)[C@H](NC(=O)OCCN(CCOC(=O)N[C@H](C(=O)N[C@@H](CCCNC(N)=O)C(=O)Nc1ccc(COC(=O)N(C)CCN(C)C(=O)OC[C@@H]2[C@@H](O)[C@H](O)[C@H](O)[C@H]3NC(=O)C(=O)N23)cc1)C(C)C)C(=O)CN1C(=O)C=CC1=O)C(=O)N[C@@H](CCCNC(N)=O)C(=O)Nc1ccc(COC(=O)N(C)CCN(C)C(=O)OC[C@@H]2[C@@H](O)[C@H](O)[C@H](O)[C@H]3NC(=O)C(=O)N23)cc1]

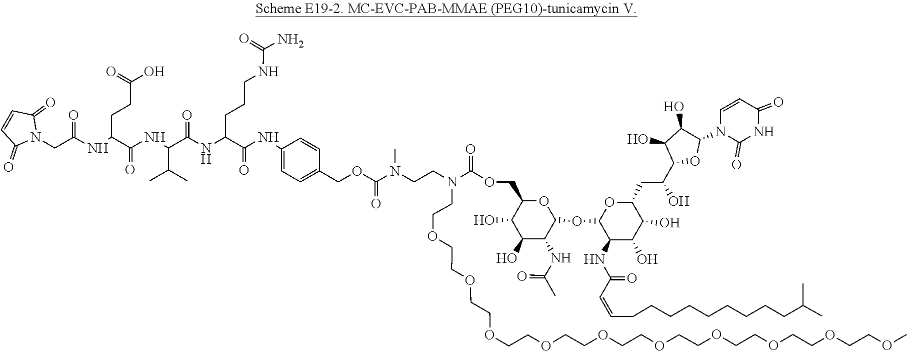

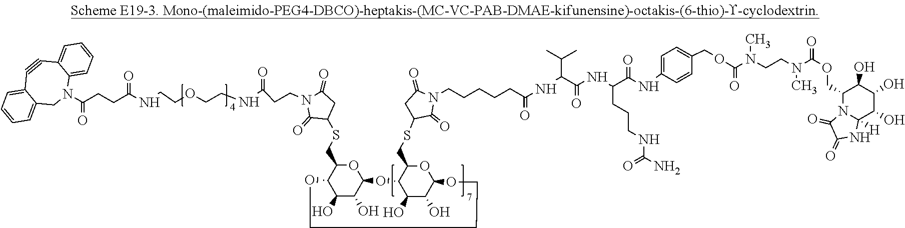

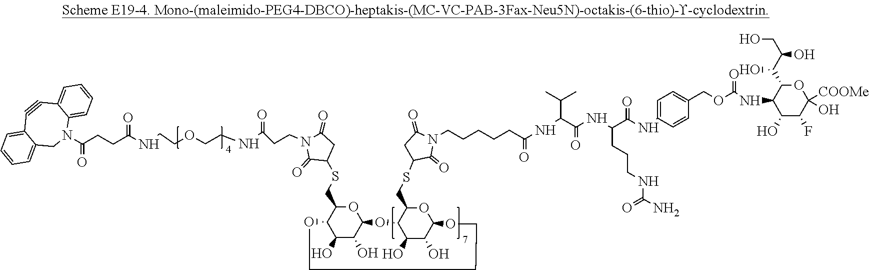

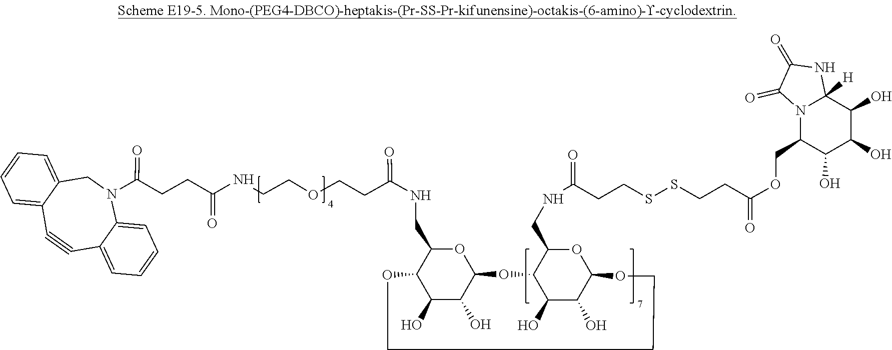

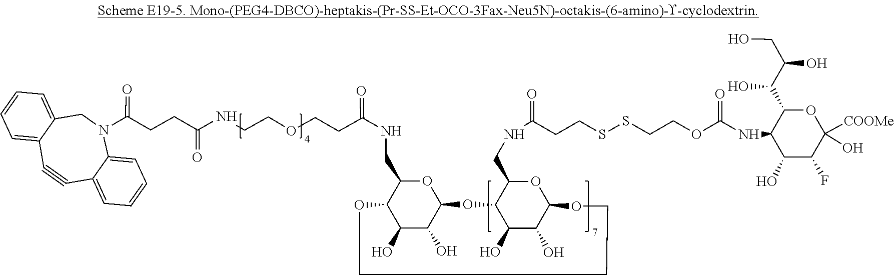

Maleimide-(VC-PAB-DMAE-kifunensine)₂ is conjugated to reduced trastuzumab and other antibodies as described above. Conjugation level is shown to be DAR=16 by Fabricator digestion and MALDI-TOF MS analysis of isolated antibody fragments as described above.

MC-EVC-PAB-MMAE(PEG10)-tunicamycin V is conjugated to reduced trastuzumab and other antibodies as described above. Conjugation level is shown to be DAR=8 by Fabricator digestion and MALDI-TOF MS analysis of isolated antibody fragments as described above. The HIC retention time is between trastuzumab and DAR=3 trastuzumab-MC-VC-PAB-MMAE ADC, when HIC-HPLC is performed as described above, thus enabling better pharmacokinetics and efficacy in vivo.

Mono-(maleimido-PEG4-DBCO)-heptakis-(MC-VC-PAB-DMAE-kifunensine)-octakis-(6-thio)-γ-cyclodextrin and mono-(maleimido-PEG4-DBCO)-heptakis-(MC-VC-PAB-3Fax-Neu5N)-octakis-(6-thio)-γ-cyclodextrin are separately conjugated to DAR=2 or DAR=4 azido-trastuzumab and other antibodies as described above to yield DAR=14 and DAR=28 conjugates, respectively.

Mono-(PEG4-DBCO)-heptakis-(Pr—SS—Pr-kifunensine)octakis-(6-amino)-γ-cyclodextrin and mono-(PEG4-DBCO)-heptakis(Pr—SS-Et-000-3Fax-Neu5N)-octakis-(6-amino)-γ-cyclodextrin are separately conjugated to DAR=2 or DAR=4 azido-trastuzumab and other antibodies as described above to yield DAR=14 and DAR=28 conjugates, respectively.

### Example 20. In Vivo Efficacy Trial

Efficacy of single dose 2.5 mg/kg trastuzumab (Herceptin, Roche), single dose 2.5 mg/kg trastuzumab-MC-VC-PAB-DMAE-tunicamycin ADC DAR=8 (tunicamycin-ADC) and repeated dose 1.5 mg/kg pembrolizumab (Keytruda, Merck) were evaluated against NCI-N87 cancer cell line tumors in vivo. The study was performed by Inovotion SAS (La Tronche, France) as follows: Fertilized chicken eggs were incubated at 37.5° C. with 50% relative humidity for 9 days (E9), when the chorioallantoic membrane (CAM) was dropped down by drilling a small hole through the eggshell into the air sac, and a 1 cm² window was cut in the eggshell above the CAM. The NCI-N87 cell line was cultivated in RPMI-1640 medium supplemented with 10% FBS and 1% penicillin/streptomycin. On day E9, cells were detached by trypsin, washed with complete medium and suspended in graft medium. An inoculum of 2 million cells was added onto the CAM of each egg. On day 10 (E10), tumors began to be detectable. Lived grafted eggs were randomized into groups and were then treated on day E10 (single dose: trastuzumab and tunicamycin-ADC), or on day E10, E11.5, E13, E14.5 and E17 (five doses: pembrolizumab) by dropping 100 μl of vehicle (PBS) and compounds (alone or in combination) onto the tumor. On day 18 (E18) the upper portion of the CAM was removed, washed in PBS and then directly transferred in PFA (fixation for 48 h). The tumors were then carefully cut away from normal CAM tissue and weighed. Eggs were checked at each treatment time, or at least every two days, for viability during the study. At the end of the study, the number of dead embryos was counted and combined with the observation of eventual visible macroscopic abnormalities (observation done during the sample collection) to evaluate the toxicity.

The results of the in vivo trial are shown in Table 2 below. There were no major differences in % alive egg embryos, and thus no different level of toxicity between the groups, and the level of % alive egg embryos was deemed normal. Compared to PBS control group, both trastuzumab (p=0.002, Students t-test) and tunicamycin-ADC (p=0.033, Students t-test) showed statistically significant difference to the control group and thus therapeutic efficacy. However, pembrolizumab alone did not show significant effect on tumor size. Compared to pembrolizumab alone, both trastuzumab+pembrolizumab (p=0.035, Students t-test) and tunicamycin-ADC+pembrolizumab treatments (p=0.023, Students t-test) showed statistically significant difference to the pembrolizumab alone group and thus therapeutic efficacy. However, the trastuzumab and tunicamycin-ADC groups (with or without pembrolizumab) did not differ from each other significantly in this model.

TABLE 2
in vivo trial results.

| | Ctrl | | | Trastuzumab + | | Tunicamycin-ADC + |
|---|---|---|---|---|---|---|
| Group | (PBS) | Pembrolizumab | Trastuzumab | Pembrolizumab | Tunicamycin-ADC | Pembrolizumab |
| Mean tumor | 32.2 | 34.9 | 17.5 | 23.6 | 22.1 | 21.4 |
| size (mg) | | | | | | |
| SEM | 2.9 | 4.7 | 2.6 | 2.4 | 3.2 | 2.9 |
| n | 10 | 7 | 8 | 10 | 8 | 9 |
| % alive | 83 | 58 | 67 | 83 | 80 | 75 |

It is obvious to a person skilled in the art that with the advancement of technology, the basic idea may be implemented in various ways. The embodiments are thus not limited to the examples described above; instead they may vary within the scope of the claims.

The embodiments described hereinbefore may be used in any combination with each other. Several of the embodiments may be combined together to form a further embodiment. A product, a method, or a use, disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1-24. (canceled)

25. A conjugate comprising:
a targeting unit for delivery to a tumour, and
a glycosylation inhibitor for inhibiting glycosylation in the tumour, thereby decreasing the immunosuppressive activity of the tumour,
wherein the glycosylation inhibitor is conjugated to the targeting unit.

26. The conjugate according to claim 25, wherein the conjugate is represented by Formula I:
[D-L]ₙ-T Formula I
wherein D is the glycosylation inhibitor, T is the targeting unit, L is a linker unit linking D to T at least partially covalently, and n is at least 1.

27. The conjugate according to claim 25, wherein the glycosylation inhibitor is selected from the group of a metabolic inhibitor, a cellular trafficking inhibitor, tunicamycin, a plant alkaloid, a substrate analog, a glycoside primer, a specific inhibitor of glycosylation, an N-acetylglucosaminylation inhibitor, a sialylation inhibitor, a fucosylation inhibitor, a galactosylation inhibitor, a mannosylation inhibitor, a mannosidase inhibitor, a glucosidase inhibitor, a glucosylation inhibitor, an N-glycosylation inhibitor, an O-glycosylation inhibitor, a glycosaminoglycan biosynthesis inhibitor, a glycosphingolipid biosynthesis inhibitor, a sulphation inhibitor, Brefeldin A, 6-diazo-5-oxo-L-norleucine, chlorate, 2-deoxyglucose, a fluorinated sugar analog, 2-acetamido-2,4-dideoxy-4-fluoroglucosamine, 2-acetamido-2,3-di deoxy-3-fluoroglucosamine, 2-acetamido-2,6-dideoxy-6-fluoroglucosamine, 2-acetamido-2,5-dideoxy-5-fluoroglucosamine, 4-deoxy-4-fluoroglucosamine, 3-deoxy-3-fluoroglucosamine, 6-deoxy-6-fluoroglucosamine, 5-deoxy-5-fluoroglucosamine, 3-deoxy-3-fluorosialic acid, 3-deoxy-3 ax-fluorosialic acid, 3-deoxy-3 eq-fluorosialic acid, 3-deoxy-3-fluoro-Neu5Ac, 3-deoxy-3 ax-fluoro-Neu5Ac, 3-deoxy-3eq-fluoro-Neu5Ac, 3-deoxy-3-fluorofucose, 2-deoxy-2-fluoroglucose, 2-deoxy-2-fluoromannose, 2-deoxy-2-fluorofucose, 3-fluorosialic acid, castanospermine, australine, deoxynojirimycin, N-butyldeoxynojirimycin, deoxymannojirimycin, kifunensin, swainsonine, mannostatin A, alloxan, streptozotocin, 2-acetamido-2,5-dideoxy-5-thioglucosamine, 2-acetamido-2,4-dideoxy-4-thioglucosamine, PUGNAc (O-[2-acetamido-2-deoxy-D-glucopyranosylidene]amino-N-phenylcarbamate), Thiamet-G, N-acetylglucosamine-thiazoline (NAG-thiazoline), GlcNAcstatin, a nucleotide sugar analog, a UDP-GlcNAc analog, a UDP-GalNAc analog, a UDP-Glc analog, a UDP-Gal analog, a GDP-Man analog, a GDP-Fuc analog, a UDP-GlcA analog, a UDP-Xyl analog, a CMP-Neu5Ac analog, a nucleotide sugar bisubstrate, a glycoside primer, an β-xyloside, an β-N-acetylgalactosaminide, an β-glucoside, an β-galactoside, β-N-acetylglucosaminide, an β-N-acetyllactosaminide, a disaccharide glycoside and a trisaccharides glycoside, 4-methyl-umbelliferone, glucosylceramide epoxide, D-threo-1-phenyl-2-decanoylamino-3-morpholino-1-propanol (PDMP), PPPP, 2-amino-2-deoxymannose, a 2-acyl-2-deoxy-glucosyl-phosphatidylinositol, 10-propoxydecanoic acid, Neu5Ac-2-ene (DANA), 4-amino-DANA, 4-guanidino-DANA, (3R, 4R, 5 S)-4-acetamido-5-amino-3-(1-ethylpropoxyl)-1-cyclohexane-1-carboxylic acid, (3R, 4R, 5 S)-4-acetamido-5-amino-3-(1-ethylpropoxyl)-1-cyclohexane-1-carboxylic acid ethyl ester, 2,6-dichloro-4-nitrophenol, pentachlorophenol, a mannosidase I inhibitor, a glucosidase I inhibitor, a glucosidase II inhibitor, an N-acetylglucosaminyltransferase inhibitor, an N-acetylgalactosaminyltransferase inhibitor, a galactosyltransferase inhibitor, a sialyltransferase inhibitor, a hexosamine pathway inhibitor, a glutamine-fructose-6-phosphate aminotransferase (GFPT1) inhibitor, a phosphoacetylglucosamine mutase (PGM3) inhibitor, a UDP-GlcNAc synthase inhibitor, a CMP-sialic acid synthase inhibitor, N-acetyl-D-glucosamine-oxazoline, 6-methyl-phosphonate-N-acetyl-D-glucosamine-oxazoline, 6-methyl-phosphonate-N-acetyl-D-glucosamine-thiazoline, V-ATPase inhibitor, a concanamycin, concanamycin A, concanamycin B, concanamycin C, a bafilomycin, bafilomycin A1, an archazolid, archazolid A, a salicylihalamide, salicylihalamide A, an oximidine, oximidine I, a lobatamide, lobatamide A, an apicularen, apicularen A, apicularen B, cruentaren, a plecomacrolide, (2Z,4E)-5-(5,6-dichloro-2-indolyl)-2-methoxy-N-(1,2,2,6,6-pentamethylpiperidin-4-yl)-2,4-pentadienamide (INDOLO), epi-kifunensine, deoxyfuconojirimycin, 1,4-dideoxy-1,4-imino-D-mannitol, 2,5-dideoxy-2,5-imino-D-mannitol, 1,4-dideoxy-1,4-imino-D-xylitol, a lysophospholipid acyltransferase (LPAT) inhibitor, a cytoplasmic phospholipase A₂ (PLA₂) inhibitor, an acyl-CoA cholesterol acyltransferase (ACAT) inhibitor, CI-976, an N-acyldeoxynojirimycin, N-acetyldeoxynojirimycin, an N-acyldeoxymannojirimycin, N-acetyldeoxymannojirimycin, a coat protein (COPI) inhibitor, a brefeldin, tamoxifen, raloxifene, sulindac, 3-deoxy-3-fluoro-Neu5N, 3-deoxy-3 ax-fluoro-Neu5N, 3-deoxy-3 eq-fluoro-Neu5N, 3′-azido-3′-deoxythymidine, 3′-fluoro-3′-deoxythymidine, 3′-azido-3′-deoxycytidine, 3′-fluoro-3′-deoxycytidine, 3′-azido-2′,3′-dideoxycytidine, 3′-fluoro-2′,3′-dideoxycytidine, and any analogs, modifications, acylated analogs, acetylated analogs, methylated analogs, or combinations thereof.

28. The conjugate according to claim 25, wherein the glycosylation inhibitor is represented by formula II: 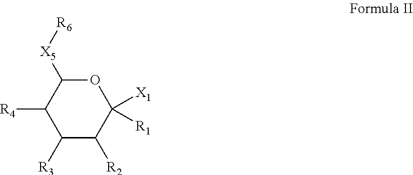
wherein X₁ is H, COOH, COOCH₃ or COOL′;
R₁ is absent, OH, OZ or L′;
R₂ is absent, Y, OH, OZ, NHCOCH₃ or L′;
R₃ is absent, Y, OH, OZ or L′;
R₄ is absent, Y, OH, OZ, NHCOCH₃ or L′;
X₅ is absent, CH₂, CH(OH)CH₂, CH(OZ)CH₂, CH(OH)CH(OH)CH₂, CH(OZ)CH(OZ)CH₂, a C₁-C₁₂ alkyl, or a substituted C₁-C₁₂ alkyl;
R₆ is OH, OZ or L′;
L′ is a bond to L;
each Z is independently selected from COCH₃, an C₁-C₁₂ acyl and a substituted C₁-C₁₂ acyl; and
Y is selected from F, Cl, Br, I, H and CH₃;
with the proviso that not more than one of R₁, R₂, R₃, R₄ and R₆ is Y, and that D contains not more than one L′; or
wherein the glycosylation inhibitor is represented by formula II, wherein
X₁ is H, COOH, COOCH₃ or COOL′;
R₁ is absent, OH, OZ or L′;
R₂ is absent, Y, OH, OZ, NHCOCH₃ or L′;
R₃ is absent, Y, OH, OZ or L′;
R₄ is absent, Y, OH, OZ, NH₂, NR₄′R₄″, NHCOCH₃ or L′;
X₅ is absent, CH₂, CH(OH)CH₂, CH(OZ)CH₂, CH(OH)CH(OH)CH₂, CH(OZ)CH(OZ)CH₂, C₁-C₁₂ alkyl, or substituted C₁-C₁₂ alkyl;
R₆ is absent, Y, OH, OZ or L′;
L′ is a bond to L;
each Z is independently selected from COCH₃, C₁-C₁₂ acyl and substituted C₁-C₁₂ acyl;
Y is selected from F, Cl, Br, I, H and CH₃; and
R₄′ and R₄″ are each independently selected from H, C₁-C₁₂ alkyl, substituted C₁-C₁₂ alkyl, C₆-C₁₂ aryl, substituted C₆-C₁₂ aryl, COR₄′″ and COOR₄′″, wherein R₄′″ is selected from C₁-C₁₂ alkyl, substituted C₁-C₁₂ alkyl, C₆-C₁₂ aryl and substituted C₆-C₁₂ aryl;
with the proviso that not more than one of R₁, R₂, R₃, R₄ and R₆ are Y, that the glycosylation inhibitor contains not more than one L′, and when one of R₄′ and R₄″ is either COR₄′″ and COOR₄′″, then one of R₄′ and R₄″ is H; or
wherein the glycosylation inhibitor is represented by formula II, wherein
X₁ is H, COOH, COOCH₃ or COOL′;
R₁ is absent, OH, OZ or L′;
R₂ is absent, Y, OH, OZ, NHCOCH₃ or L′;
R₃ is absent, Y, OH, OZ or L′;
R₄ is absent, Y, OH, OZ, NH₂, NR₄′R₄″, NHCOCH₃ or L′;
X₅ is absent, CH₂, CH(OH)CH₂, CH(OZ)CH₂, CH(OH)CH(OH)CH₂, CH(OZ)CH(OZ)CH₂, a C₁-C₁₂ alkyl, or a substituted C₁-C₁₂ alkyl;
R₆ is absent, Y, OH, OZ or L′;
L′ is a bond to L;
each Z is independently selected from COCH₃, a C₁-C₁₂ acyl and a substituted C₁-C₁₂ acyl; and
Y is selected from F, Cl, Br, I, H and CH₃; and
R₄′ and R₄″ are each independently selected from H, C₁-C₁₂ alkyl, substituted C₁-C₁₂ alkyl, C₆-C₁₂ aryl, substituted C₆-C₁₂ aryl, COR₄′″ and COOR₄′″, wherein R₄′″ is selected from C₁-C₁₂ alkyl, substituted C₁-C₁₂ alkyl, C₆-C₁₂ aryl and substituted C₆-C₁₂ aryl;
with the proviso that two of R₁, R₂, R₃, R₄ and R₆ are Y, that the glycosylation inhibitor contains not more than one L′, and when one of R₄′ and R₄″ is either COR₄′″ or COOR₄′″, then one of R₄′ and R₄″ is H; or
wherein the glycosylation inhibitor is represented by formula II, wherein
X₁ is H, COOH, COOCH₃ or COOL′;
R₁ is absent, OH, OZ or L′;
R₂ is absent, Y, OH, OZ, NHCOCH₃ or L′;
R₃ is absent, Y, OH, OZ or L′;
R₄ is absent, Y, OH, OZ, NH₂, NR₄′R₄″, NHCOCH₃ or L′;
X₅ is absent, CH₂, CH(OH)CH₂, CH(OZ)CH₂, CH(OH)CH(OH)CH₂, CH(OZ)CH(OZ)CH₂, a C₁-C₁₂ alkyl, or a substituted C₁-C₁₂ alkyl;
R₆ is absent, Y, OH, OZ or L′;
L′ is a bond to L;
each Z is independently selected from COCH₃, a C₁-C₁₂ acyl and a substituted C₁-C₁₂ acyl;
Y is selected from F, Cl, Br, I, H and CH₃; and
R₄′ and R₄″ are each independently selected from H, C₁-C₁₂ alkyl, substituted C₁-C₁₂ alkyl, C₆-C₁₂ aryl, substituted C₆-C₁₂ aryl, COR₄′″ and COOR₄′, wherein R₄′″ is selected from C₁-C₁₂ alkyl, substituted C₁-C₁₂ alkyl, C₆-C₁₂ aryl and substituted C₆-C₁₂ aryl;
with the proviso that three of R₁, R₂, R₃, R₄ and R₆ are Y, that the glycosylation inhibitor contains not more than one L′, and when one of R₄′ and R₄″ is either COR₄′″ and COOR₄′″, then one of R₄′ and R₄″ is H.

29. The conjugate according to claim 25, wherein the glycosylation inhibitor is represented by any one of Formulas IIIa, IIIb, IIIc, IIId, IIIe, IIIf, IIIg or IIIh: 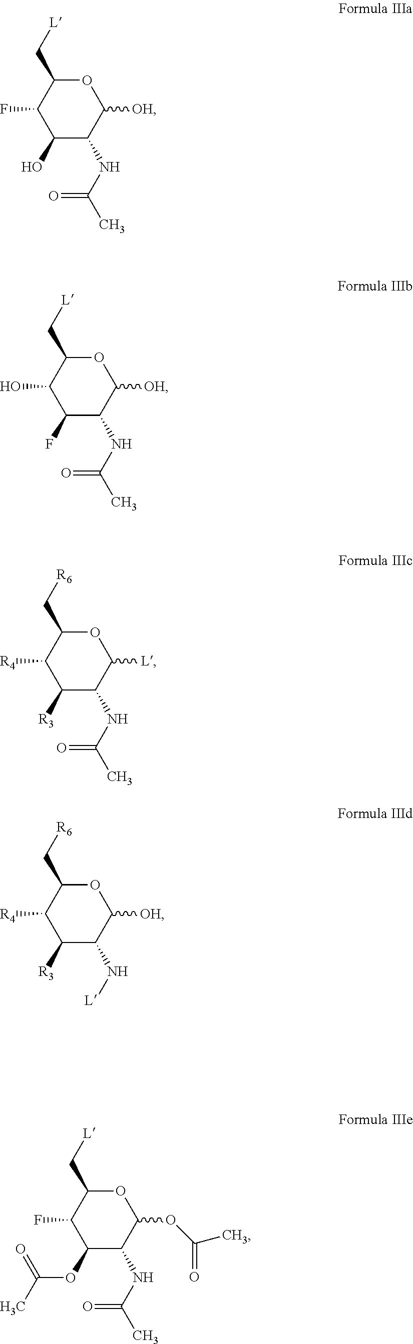 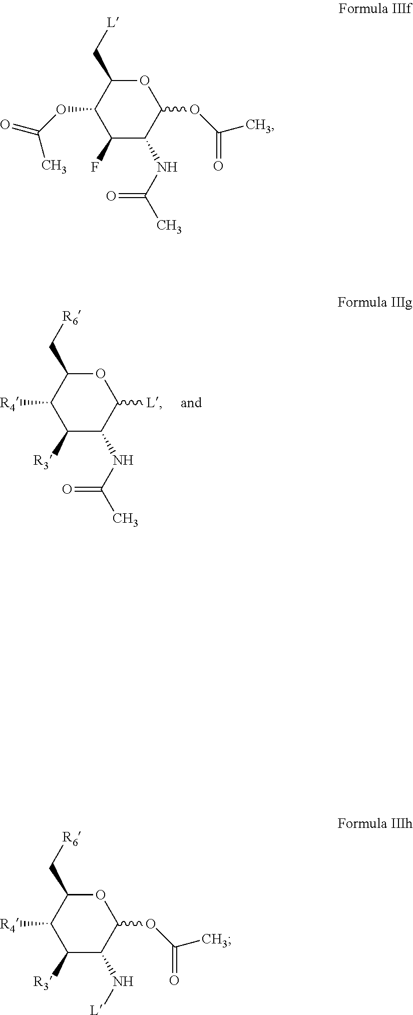
wherein
L′ is a bond to L;
R₃, R₄ and R₆ are each independently either OH or F, with the proviso that only one of R₃, R₄ and R₆ is F; and
R₃′, R₄′ and R₆′ are each independently either COCH₃ or F, with the proviso that only one of R₃′, R₄′ and R₆′ is F; or
wherein the glycosylation inhibitor is represented by any one of formulas IIIa, IIIb, IIIc, IIId, IIIe, IIIf, IIIg or IIIb;
wherein
L′ is a bond to L;
R₃, R₄ and R₆ are each independently either OH or F, with the proviso that two of R₃, R₄ and R₆ are F; and
R₃′, R₄′ and R₆′ are each independently either OCOCH₃ or F, with the proviso that two of R₃′, R₄′ and R₆′ are F; or
wherein the glycosylation inhibitor is represented by any one of formulas IIIa, IIIb, IIId, IIIe, IIIf, IIIg or IIIb, wherein:
L′ is a bond to L;
R₃, R₄ and R₆ are each F; and
R₃′, R₄′ and R₆′ are each F;
or wherein the glycosylation inhibitor is a 3-deoxy-3-fluorosialic acid represented by any one of Formulas IVa, IVb, IVc, IVd, IVe, IVf, IVg or IVh: 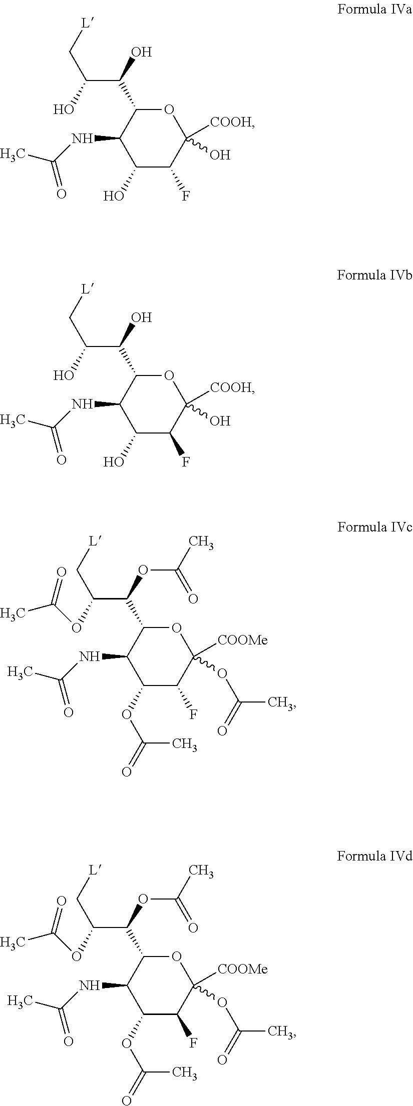 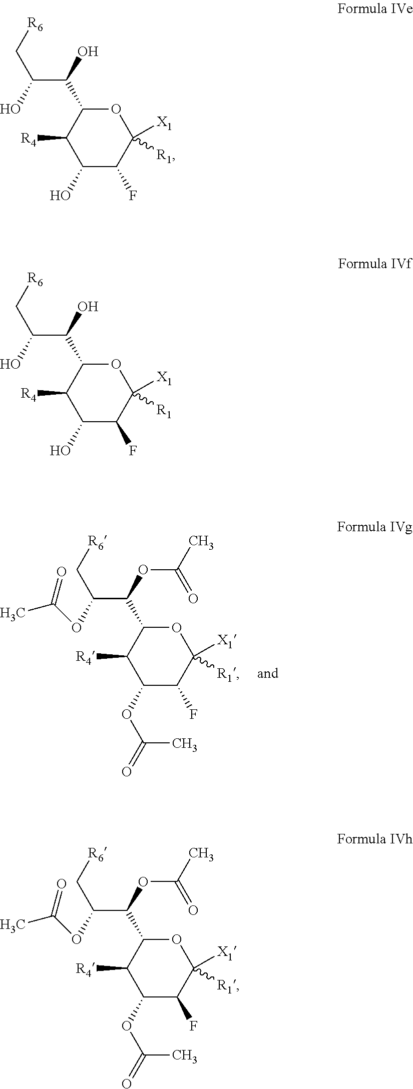
wherein:
L′ is a bond to L;
R₁ and R₆ are each independently either OH or L′, R₄ is independently either NHCOCH₃ or L′, and X₁ is independently either COOH or L′, with the proviso that only one of R₁, R₄, R₆ and X₁ is L′; and
R₁′ and R₆′ are each independently either OCOCH₃ or L′, R₄′ is independently either NHCOCH₃ or L′, and X₁′ is independently either COOCH₃ or L′,
with the proviso that only one of R₁′, R₄′, R₆′ and X₁′ is L′; or wherein
the glycosylation inhibitor is a 3-deoxy-3-fluorosialic acid represented by any one of formulas IVe, IVf, IVg or IVh, wherein
L′ is a bond to L;
R₁ and R₆ are each independently either OH, OZ or L′;
R₄ and R₄′ are independently either absent, OH, OZ, NH₂, NR₄″R₄″′, NHL′, NHCOCH₃ or L′;
X₁ is independently either COOH, COOMe, COOL′ or L′;
each Z is independently selected from COCH₃, a C₁-C₁₂ acyl and a substituted C₁-C₁₂ acyl;
R₁′ and R₆′ are each independently either OH, OZ, OCOCH₃ or L′;
R₄″ and R₄′″ are each independently selected from H, C₁-C₁₂ alkyl, substituted C₁-C₁₂ alkyl, C₆-C₁₂ aryl, substituted C₆-C₁₂ aryl, COR₄″″ and COOR₄″″, L′, L″-L′, Y, NH₂, OH, NHCOCH₃, NHCOCH₂OH, NHCOCF₃, NHCOCH₂Cl, NHCOCH₂OCOCH₃, NHCOCH₂N₃, NHCOCH₂CH₂CCH, NHCOOCH₂CCH, NHCOOCH₂CHCH₂, NHCOOCH₃, NHCOOCH₂CH₃, NHCOOCH₂CH(CH₃)₂, NHCOOC(CH₃)₃, NHCOO-benzyl, NHCOOCH₂-1-benzyl-1H-1,2,3-triazol-4-yl, NHCOO(CH₂)₃CH₃, NHCOO(CH₂)₂OCH₃, NHCOOCH₂CCl₃ and NHCOO(CH₂)₂F (wherein benzyl=CH₂C₆H₅);
wherein R₄″″ is selected from C₁-C₁₂ alkyl, substituted C₁-C₁₂ alkyl, C₆-C₁₂ aryl and substituted C₆-C₁₂ aryl;
L″ is selected from L′-substituted C₁-C₁₂ alkyl, L′-substituted C₆-C₁₂ aryl, COL′″, COOL′″, NH—, O—, NHCOCH₂—, NHCOCH₂O—, NHCOCF₂—, NHCOCH₂OCOCH₂—, NHCOCH₂triazolyl-, NHCOOCH₂CHCH—, NHCOOCH₂CH₂CH₂S—, NHCOOCH₂—, NHCOOCH₂CH₂—, NHCOOCH₂CHCH₂CH₂—, NHCOO-benzyl-, NHCOO(CH₂)₃CH₂—, NHCOOCH₂-1-benzyl-1H-1,2,3-triazol-4-yl- and NHCOO(CH₂)₂OCH₂— (wherein benzyl is CH₂C₆H₅ and - is the bond to L′);
wherein L′″ is either L′-substituted C₁-C₁₂ alkyl or L′-substituted C₆-C₁₂ aryl,
with the proviso that the glycosylation inhibitor contains not more than one L′, and when R₄′ is either COR₄′″ or COOR₄′″ then R₄″ is H, and when R₄″ is either COR₄′″ or COOR₄′″ then R₄′ is H; or wherein
the glycosylation inhibitor is a 3-deoxy-3-fluorosialic acid represented by any one of Formulas IVi, IVj, IVk, IVl or IVm: 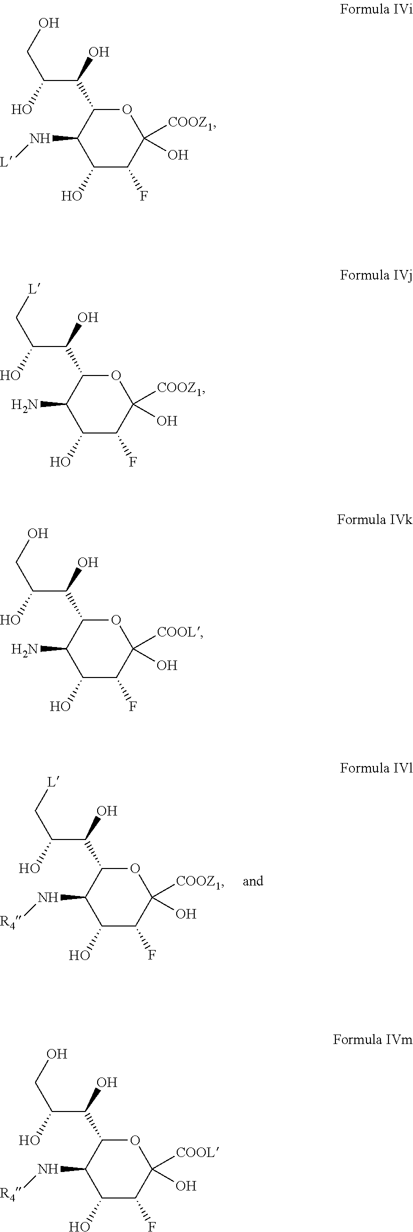
wherein
L′ is a bond to L;
Z₁ is selected from H, CH₃, C₁-C₁₂ alkyl, substituted C₁-C₁₂ alkyl, C₆-C₁₂ aryl and substituted C₆-C₁₂ aryl; and
R₄″ is selected from C₁-C₁₂ alkyl, substituted C₁-C₁₂ alkyl, C₆-C₁₂ aryl, substituted C₆-C₁₂ aryl, COR₄″″, COOR₄″″, COCH₃, COCH₂OH, COCF₃, COCH₂Cl, COCH₂OCOCH₃, COCH₂N₃, COCH₂CH₂CCH, COOCH₂CCH, COOCH₂CHCH₂, COOCH₃, COOCH₂CH₃, COOCH₂CH(CH₃)₂, COOC(CH₃)₃, COO-benzyl, COOCH₂-1-benzyl-1H-1,2,3-triazol-4-yl, COO(CH₂)₃CH₃, COO(CH₂)₂OCH₃, COOCH₂CCl₃ and COO(CH₂)₂F (wherein benzyl=CH₂C₆H₅); and,
wherein R₄″″ is selected from C₁-C₁₂ alkyl, substituted C₁-C₁₂ alkyl, C₆-C₁₂ aryl and substituted C₆-C₁₂ aryl.

30. The conjugate according to claim 25, wherein the glycosylation inhibitor is represented by Formula A: 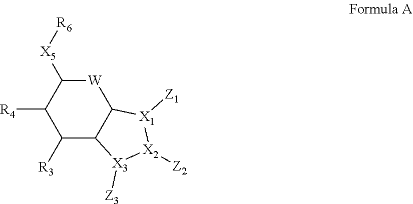
wherein
W is CH₂, NH, O or S;
X₁, X₂ and X₃ are each independently selected from S, O, C, CH and N;
with the proviso that when one or both of X₁ and X₃ are either O or S, then X₂ is either absent, a bond between X₁ and X₂, or CH;
Z₁, Z₂ and Z₃ are each independently either absent or selected from H, OH, OZ, ═O, (═O)₂, C₁-C₁₂ alkyl, substituted C₁-C₁₂ alkyl, C₆-C₁₂ aryl, substituted C₆-C₁₂ aryl or L′;
R₃ and R₄ are are each independently either absent or selected from H, OH, OZ or L′;
X₅ is absent, OH, OZ, O, CH₂, C₁-C₁₂ alkyl, or substituted C₁-C₁₂ alkyl;
R₆ is absent, H, OH, OZ, a phosphate, a phosphate ester, a phosphate analog, a boronophosphate, a boronophosphate ester, a thiophosphate, a thiophosphate ester, a halophosphate, a halophosphate ester, a vanadate, a phosphonate, a phosphonate ester, a thiophosphonate, a thiophosphonate ester, a halophosphonate, a halophosphonate ester, methylphosphonate, methylphosphonate ester or L′;
L′ is a bond to L;
each Z is independently selected from COCH₃, C₁-C₁₂ acyl and substituted C₁-C₁₂ acyl; and
each of the bonds between the ring carbon and X₃, X₂ and X₃, X₁ and X₂, and the ring carbon and X₁, are independently either a single bond or a double bond or absent;
with the proviso than when both of the bonds between X₂ and X₃, and X₁ and X₂, are absent, then both X₂ and Z₂ are also absent; and
with the proviso that the glycosylation inhibitor contains not more than one L′.

31. The conjugate according to claim 25, wherein the glycosylation inhibitor is represented by any one of Formulas Aa, Ab, Ac or Ad: 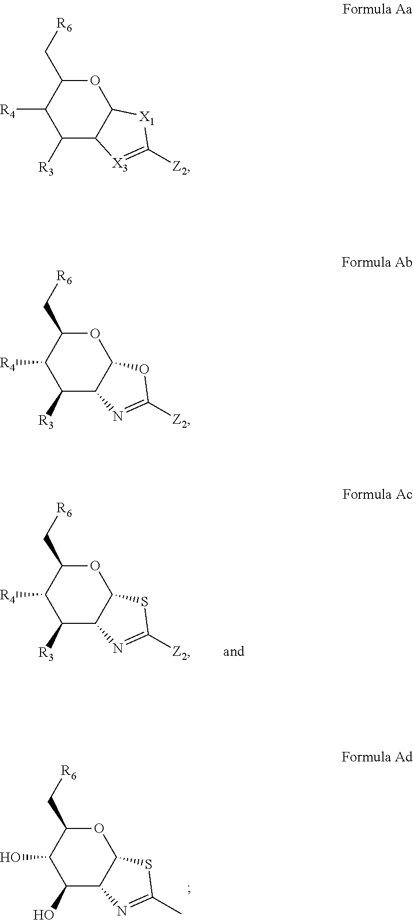
wherein
X₁ is selected from S, O, CH₂ and NH;
X₃ is selected from CH and N;
Z₂ is either absent or selected from H, OH, OZ, ═O, (═O)₂, C₁-C₁₂ alkyl, substituted C₁-C₁₂ alkyl, C₆-C₁₂ aryl, substituted C₆-C₁₂ aryl or L′;
R₃ and R₄ are are each independently either absent or selected from H, OH, OZ or L′;
R₆ is absent, H, OH, OZ, a phosphate, a phosphate ester, a phosphate analog, a thiophosphate, a thiophosphate ester, a halophosphate, a halophosphate ester, a vanadate, a phosphonate, a phosphonate ester, a thiophosphonate, a thiophosphonate ester, a halophosphonate, a halophosphonate ester, methylphosphonate, methylphosphonate ester or L′;
L′ is a bond to L; and
each Z is independently selected from COCH₃, C₁-C₁₂ acyl and substituted C₁-C₁₂ acyl;
with the proviso that the glycosylation inhibitor contains not more than one L′.

32. The conjugate according to claim 25, wherein the glycosylation inhibitor is represented by Formula B: 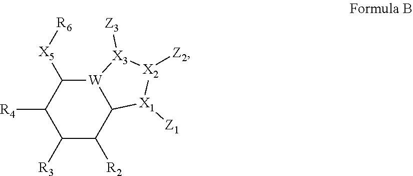
wherein
W is CH, N, O or S;
X₁, X₂ and X₃ are each independently selected from S, O, CH and N;
with the proviso that when one or both of X₁ and X₃ are either O or S, then X₂ is either absent, a bond between X₁ and X₃, C or CH;
Z₁, Z₂ and Z₃ are each independently either absent or selected from H, OH, OZ, ═O, (═O)₂, C₁-C₁₂ alkyl, substituted C₁-C₁₂ alkyl, C₆-C₁₂ aryl, substituted C₆-C₁₂ aryl or L′;
R₂, R₃ and R₄ are are each independently either absent or selected from H, OH, OZ or L′;
X₅ is absent, OH, OZ, O, CH₂, C₁-C₁₂ alkyl, or substituted C₁-C₁₂ alkyl;
R₆ is absent, H, OH, OZ or L′;
L′ is a bond to L;
each Z is independently selected from COCH₃, C₁-C₁₂ acyl and substituted C₁-C₁₂ acyl; and
each of the bonds between W and X₃, X₂ and X₃, X₁ and X₂, and the ring carbon and X₁, are independently either a single bond or a double bond or absent;
with the proviso than when both of the bonds between X₂ and X₃, and X₁ and X₂, are absent, then both X₂ and Z₂ are also absent; and
with the proviso that the glycosylation inhibitor contains not more than one L′.

33. The conjugate according to claim 25, wherein the glycosylation inhibitor is represented by any one of Formulas Ba, Bb, Bc, Bd, Be, Bf, Bg or Bh: 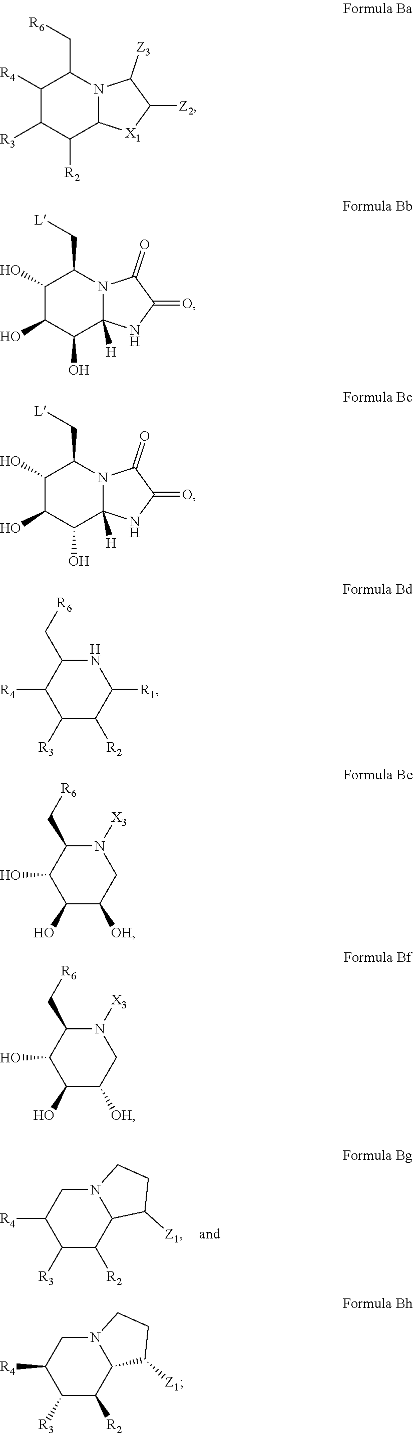
wherein
X₁ is selected from S, O, CH₂ and NH;
X₃ is selected from H, C₁-C₁₂ alkyl, substituted C₁-C₁₂ alkyl, C₁-C₁₂ acyl, substituted C₁-C₁₂ acyl, C₆-C₁₂ aryl, substituted C₆-C₁₂ aryl or L′;
Z₁, Z₂ and Z₃ are each independently either absent or selected from H, OH, OZ, ═O, (═O)₂, C₁-C₁₂ alkyl, substituted C₁-C₁₂ alkyl, C₆-C₁₂ aryl, substituted C₆-C₁₂ aryl or L′;
R₁, R₂, R₃ and R₄ are are each independently either absent or selected from H, OH, OZ or L′;
R₆ is absent, H, OH, OZ or L′;
L′ is a bond to L; and
each Z is independently selected from COCH₃, C₁-C₁₂ acyl and substituted C₁-C₁₂ acyl;
with the proviso that the glycosylation inhibitor contains not more than one L′.

34. The conjugate according to claim 25, wherein the glycosylation inhibitor is represented by any one of Formulas Ca, Cb or Cc: 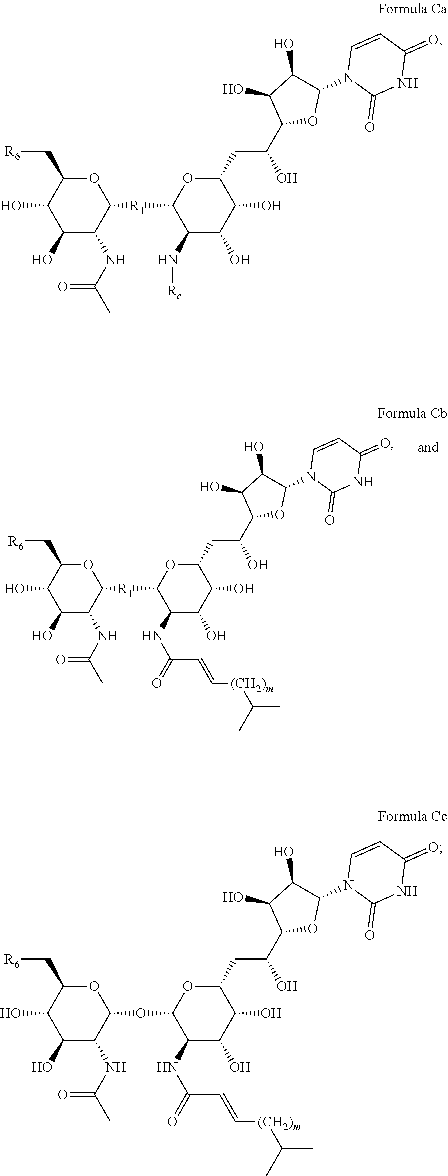
wherein
R₁ is O, NH, NRb, S, SO, SO₂ or CH₂;
Rb is C₁-C₁₀ alkyl, substituted C₁-C₁₀ alkyl, C₁-C₁₀ acyl or substituted C₁-C₁₀ acyl;
R₆ is OH or L′;
Rc is C₂-C₂₀ acyl, substituted C₂-C₂₀ acyl, C₆-C₂₀ aryl, substituted C₆-C₂₀ aryl or L′;
m is 6, 7, 8, 9, 10, 11, 12, 13 or 14; and
L′ is a bond to L.

35. The conjugate according to claim 25, wherein the glycosylation inhibitor is represented by any one of Formulas Da, Db or Dc: 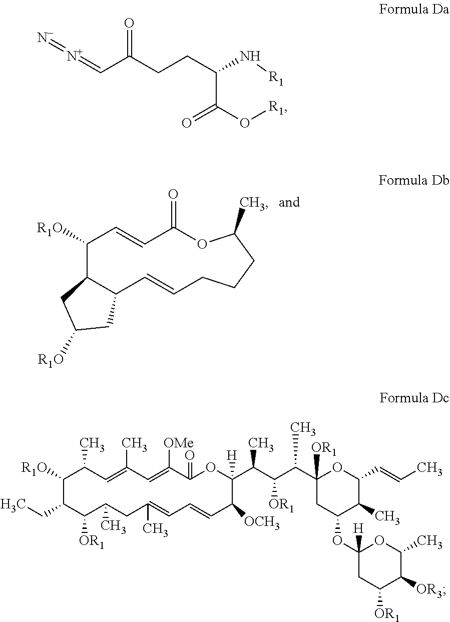
wherein
each R₁ is independently either H or L′;
R₃ is H, OH, CONH₂, CONHL′ or L′; and
L′ is a bond to L;
with the proviso that each of the Formulas Da, Db and Dc contains only one L′.

36. The conjugate according to claim 25, wherein the targeting unit comprises or is an antibody, such as a tumour cell-targeting antibody, a cancer-targeting antibody and/or an immune cell-targeting antibody; a peptide; an aptamer; or a glycan.

37. The conjugate according to claim 25, wherein the conjugate is selected from the group consisting of conjugates represented by Formulas Va-c, VIa-b, VIIa-b or VIIIa-t: 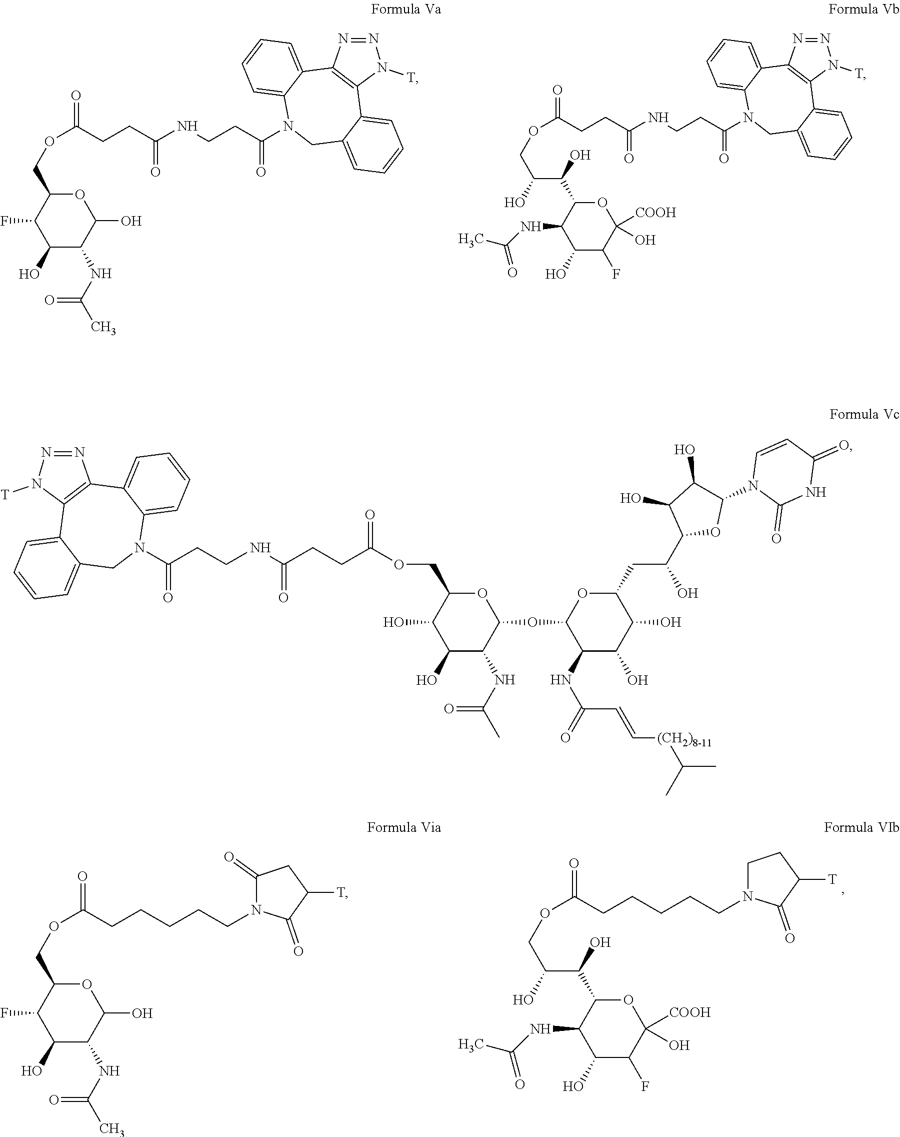 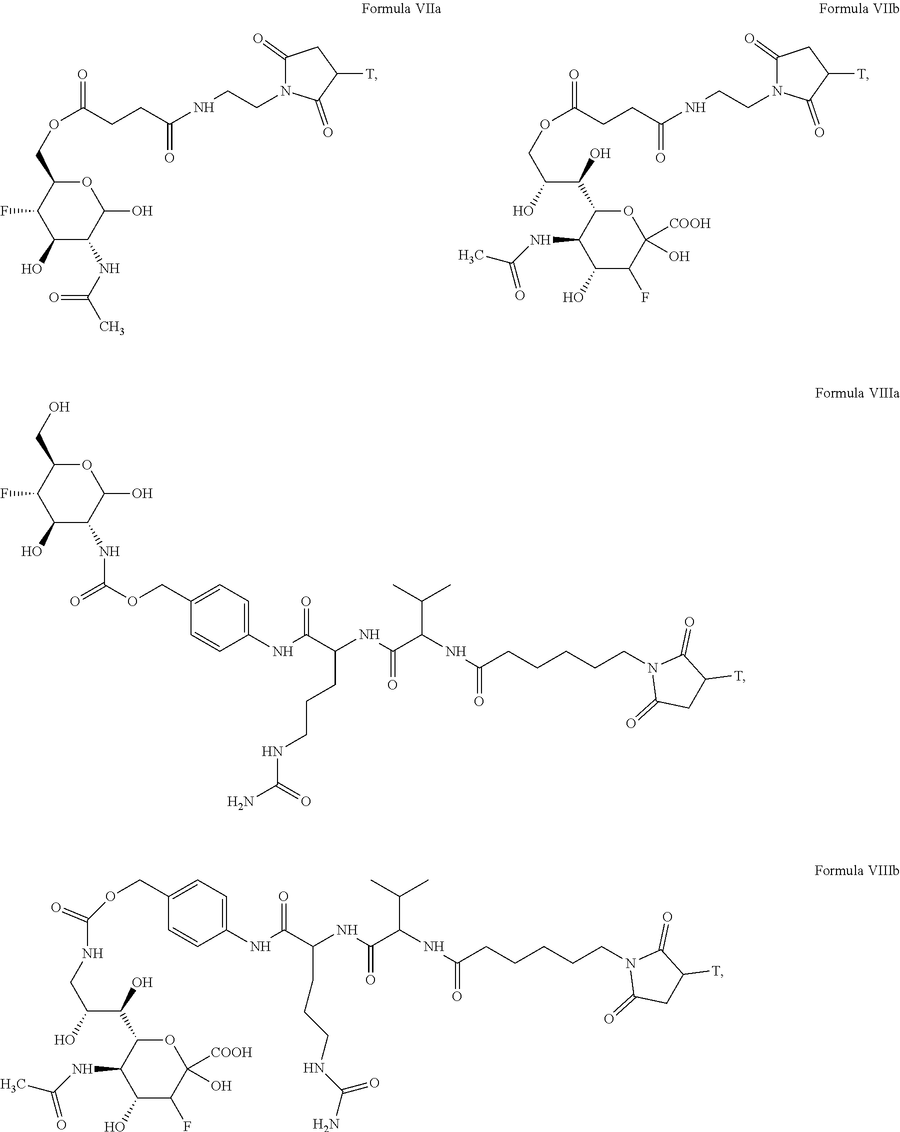 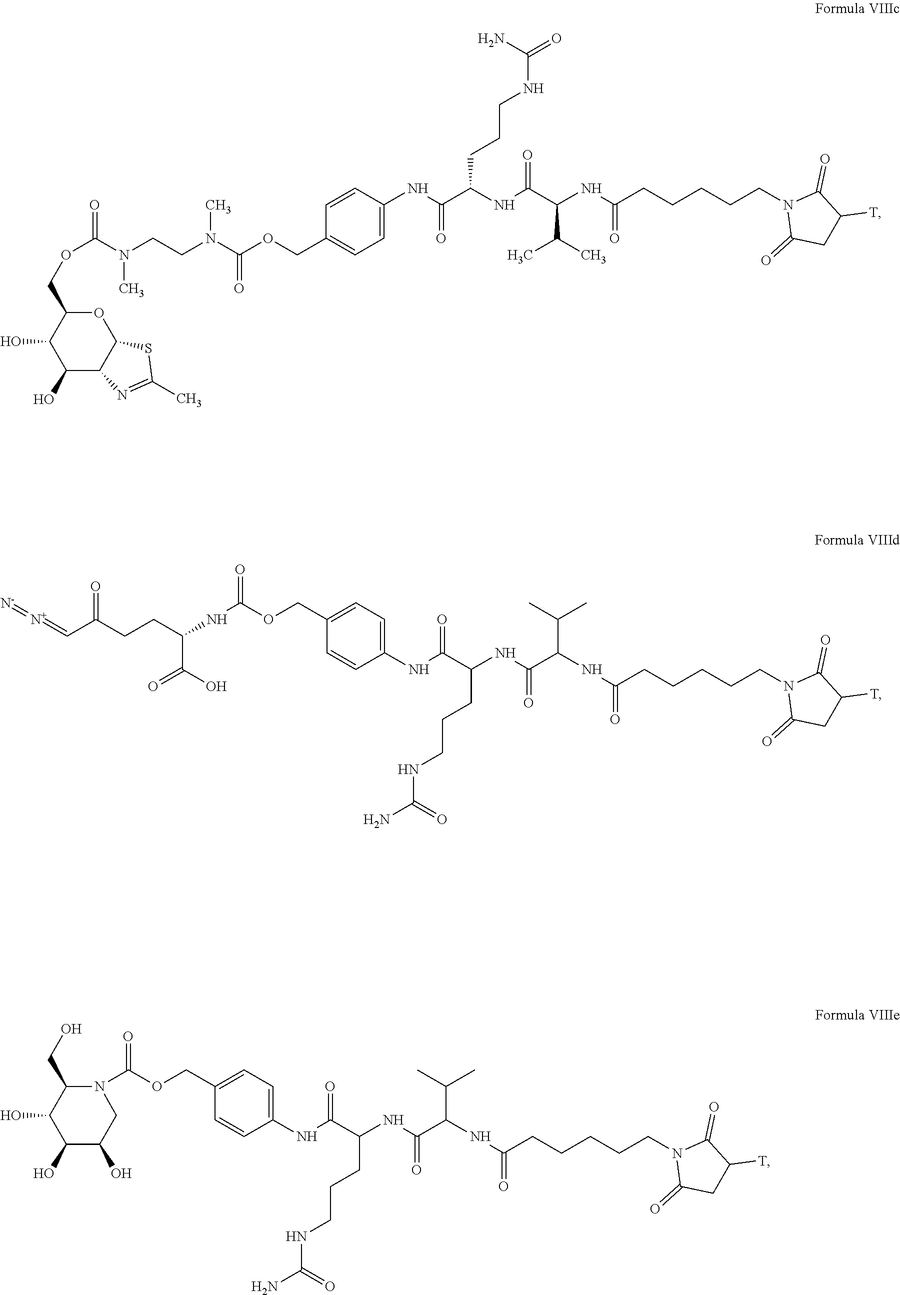 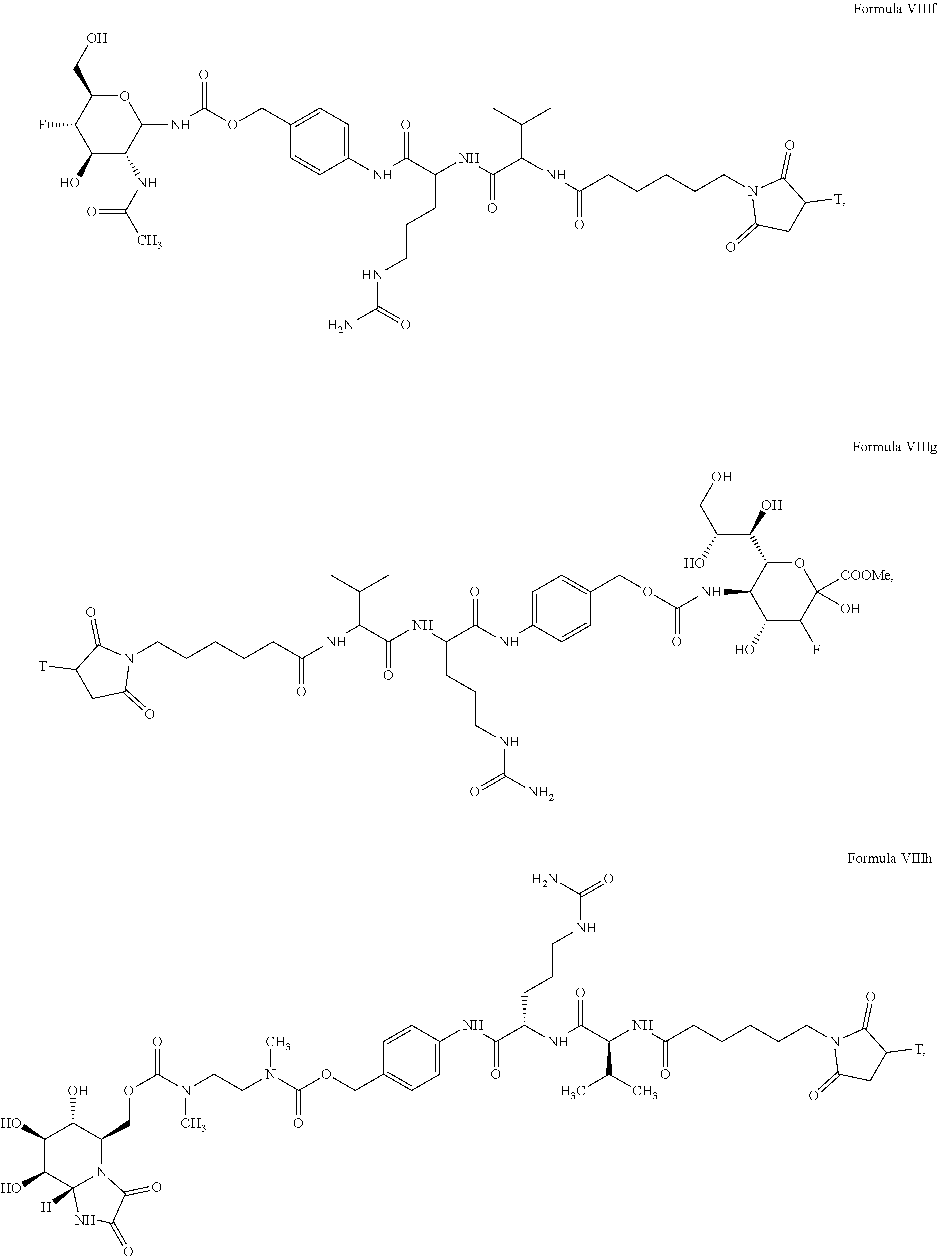 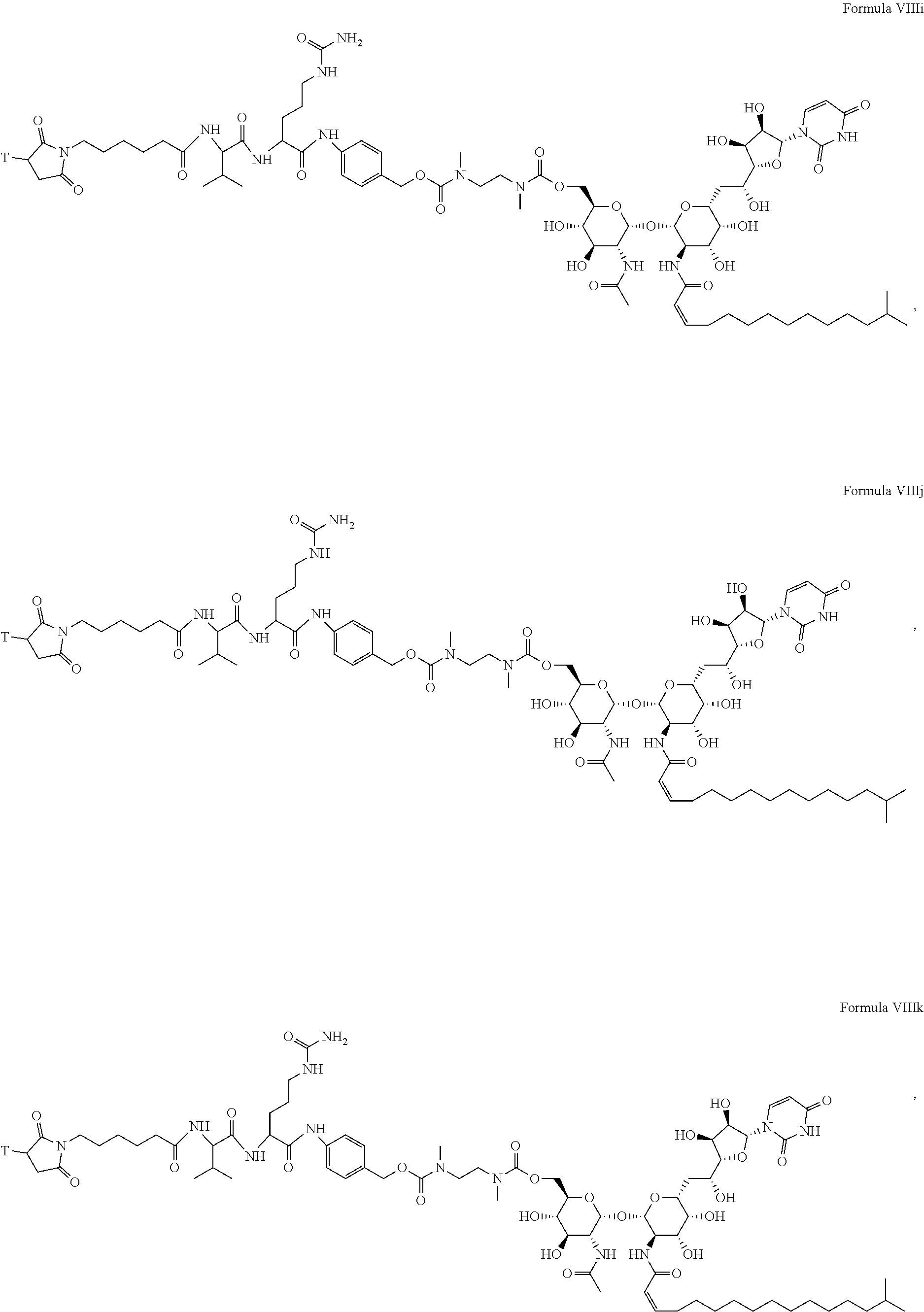 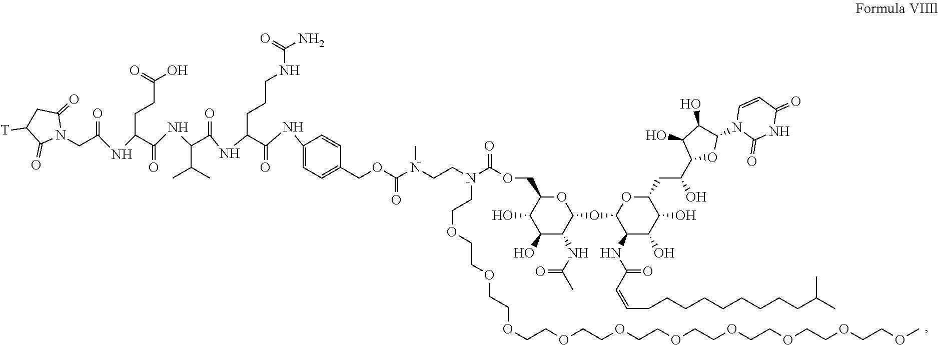 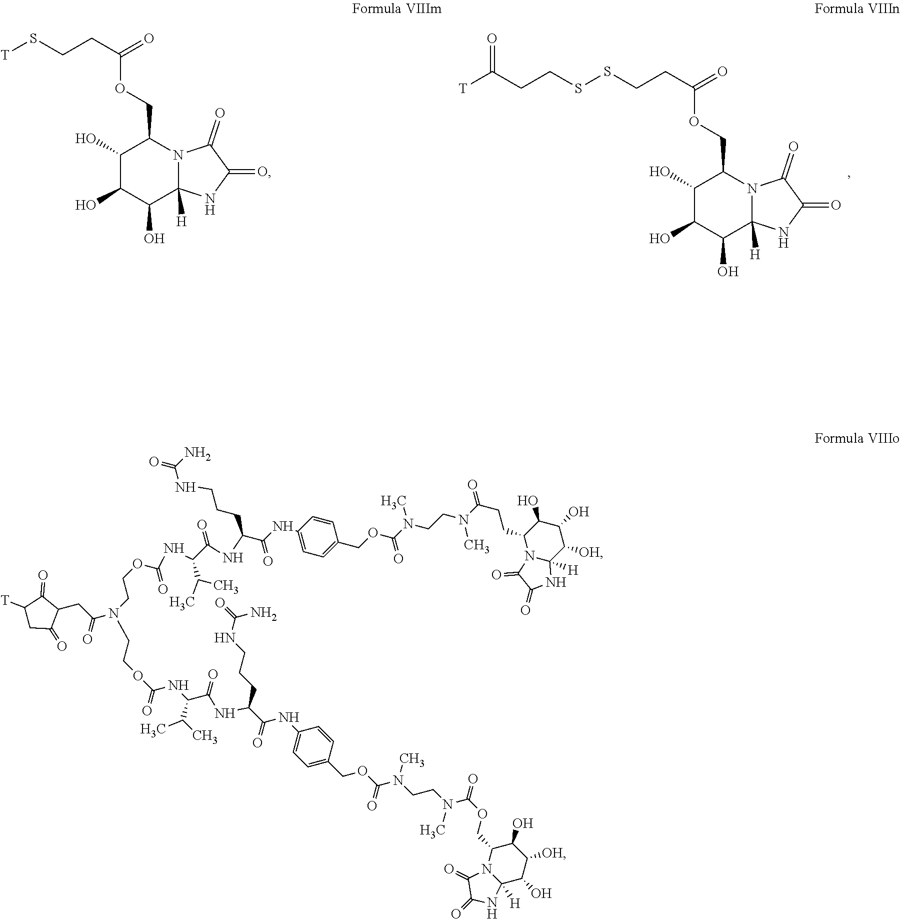 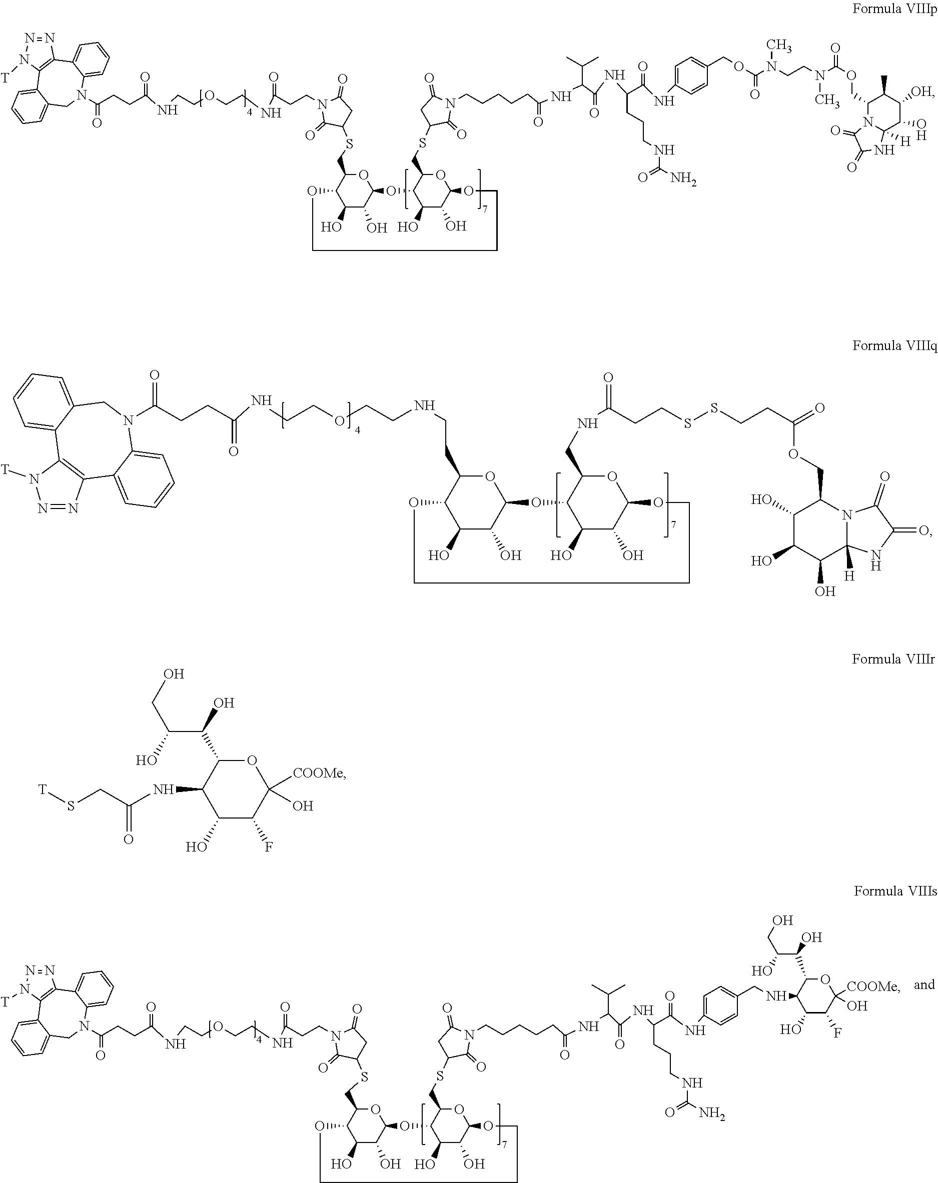 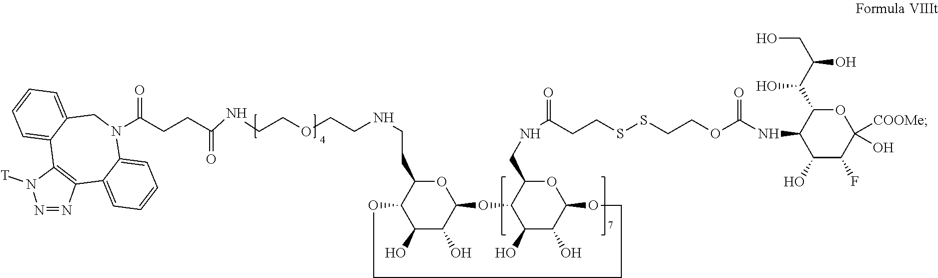
wherein T represents the targeting unit.

38. The conjugate according to claim 25, wherein:
the targeting unit comprises or is a cancer-targeting antibody selected from the group of bevacizumab, tositumomab, etanercept, trastuzumab, adalimumab, alemtuzumab, gemtuzumab ozogamicin, efalizumab, rituximab, infliximab, abciximab, basiliximab, palivizumab, omalizumab, daclizumab, cetuximab, panitumumab, epratuzumab, 2G12, lintuzumab, nimotuzumab and ibritumomab tiuxetan, or an antibody selected from the group of an anti-EGFR1 antibody, an epidermal growth factor receptor 2 (HER2/neu) antibody, an anti-CD22 antibody, an anti-CD30 antibody, an anti-CD33 antibody, an anti-Lewis y antibody, an anti-CD20 antibody, an anti-CD3 antibody, an anti-PSMA antibody, an anti-TROP2 antibody and an anti-AXL antibody; or
the targeting unit comprises or is an immune receptor-targeting antibody selected from the group of nivolumab, pembrolizumab, ipilimumab, atezolizumab, avelumab, durvalumab, BMS-986016, LAG525, MBG453, OMP-31M32, JNJ-61610588, enoblituzumab (MGA271), MGD009, 8H9, MEDI9447, M7824, metelimumab, fresolimumab, IMC-TR1 (LY3022859), lerdelimumab (CAT-152), LY2382770, lirilumab, IPH4102, 9B12, MOXR 0916, PF-04518600 (PF-8600), MEDI6383, MEDI0562, MEDI6469, INCAGN01949, GSK3174998, TRX-518, BMS-986156, AMG 228, MEDI1873, MK-4166, INCAGN01876, GWN323, JTX-2011, GSK3359609, MEDI-570, utomilumab (PF-05082566), urelumab, ARGX-110, BMS-936561 (MDX-1203), varlilumab, CP-870893, APX005M, ADC-1013, lucatumumab, Chi Lob 7/4, dacetuzumab, SEA-CD40, RO7009789, MEDI9197; or
the targeting unit comprises or is a molecule selected from the group of an immune checkpoint inhibitor, an anti-immune checkpoint molecule, anti-PD-1, anti-PD-L1 antibody, anti-CTLA-4 antibody, a cancer-targeting molecule, or a targeting unit capable of binding an immune checkpoint molecule, the immune checkpoint molecule being selected from the group of: lymphocyte activation gene-3 (LAG-3, CD223), T cell immunoglobulin-3 (TIM-3), poly-N-acetyllactosamine, T (Thomsen-Friedenreich antigen), Globo H, Lewis c (type 1 N-acetyllactosamine), Galectin-1, Galectin-2, Galectin-3, Galectin-4, Galectin-5, Galectin-6, Galectin-7, Galectin-8, Galectin-9, Galectin-10, Galectin-11, Galectin-12, Galectin-13, Galectin-14, Galectin-15, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, Siglec-17, phosphatidyl serine, CEACAM-1, T cell immunoglobulin and ITIM domain (TIGIT), CD155 (poliovirus receptor-PVR), CD112 (PVRL2, nectin-2), V-domain Ig suppressor of T cell activation (VISTA, also known as programmed death-1 homolog, PD-1H), B7 homolog 3 (B7-H3, CD276), adenosine A2a receptor (A2aR), CD73, B and T cell lymphocyte attenuator (BTLA, CD272), herpes virus entry mediator (HVEM), transforming growth factor (TGF)-β, killer immunoglobulin-like receptor (KIR, CD158), KIR2DL1/2L3, KIR3DL2, phosphoinositide 3-kinase gamma (PI3Kγ), CD47, OX40 (CD134), Glucocorticoid-induced TNF receptor family-related protein (GITR), GITRL, Inducible co-stimulator (ICOS), 4-1BB (CD137), CD27, CD70, CD40, CD154, indoleamine-2,3-dioxygenase (IDO), toll-like receptors (TLRs), TLR1, TLR2, TLR3, TLR4, TLR5, TLR6, TLR7, TLR8, TLR9, interleukin 12 (IL-12), IL-2, IL-2R, CD122 (IL-2Rβ), CD132 (γ_{c}), CD25 (IL-2Rα), and arginase.

39. The conjugate according to claim 26, wherein n is in the range of 1 to about 20, or 1 to about 15, or 1 to about 10, or 2 to 10, or 2 to 6, or 2 to 5, or 2 to 4, or 3 to about 20, or 3 to about 15, or 3 to about 10, or 3 to about 9, or 3 to about 8, or 3 to about 7, or 3 to about 6, or 3 to 5, or 3 to 4, or 4 to about 20, or 4 to about 15, or 4 to about 10, or 4 to about 9, or 4 to about 8, or 4 to about 7, or 4 to about 6, or 4 to 5; or about 7-9; or about 8, or 1, 2, 3, 4, 5, 6, 7, 8, 9, 10, 11, 12, 13, 14, 15, 16, 17, 18, 19, or 20; or in the range of 1 to about 1000, or 1 to about 2000, or 1 to about 400, or 1 to about 200, or 1 to about 100; or 100 to about 1000, or 200 to about 1000, or 400 to about 1000, or 600 to about 1000, or 800 to about 1000; 100 to about 800, or 200 to about 600, or 300 to about 500; or 20 to about 200, or 30 to about 150, or 40 to about 120, or 60 to about 100; over 8, over 16, over 20, over 40, over 60, over 80, over 100, over 120, over 150, over 200, over 300, over 400, over 500, over 600, over 800, or over 1000; or n is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 52, 54, 56, 58, 60, 62, 63, 64, 66, 68, 70, 72, 74, 76, 78, 80, 82, 84, 86, 88, 90, 92, 94, 96, 98, 100, 110, 120, 130, 140, 150, 160, 170, 180, 190, 200, 220, 240, 260, 280, 300, 350, 400, 450, 500, 550, 600, 650, 700, 750, 800, 850, 900, 950, 1000, 1100, 1200, 1300, 1400, 1500, 1600, 1700, 1800, 1900, 2000, or greater than 2000.

40. The conjugate according to claim 26, wherein L is represented by Formula IX
—R₇-L₁-Sₚ-L₂-R₈— Formula IX
wherein
R₇ is a group covalently bonded to the glycosylation inhibitor;
L₁ is a spacer unit or absent;
Sₚ is a specificity unit or absent;
L₂ is a stretcher unit covalently bonded to the targeting unit or absent; and
R₈ is absent or a group covalently bonded to the targeting unit.

41. The conjugate according to claim 40, wherein R₇ is selected from:
—C(═O)NH—,
—C(═O)O—,
—HC(═O)—,
—OC(═O)—,
—OC(═O)O—,
—NHC(═O)O—,
—OC(═O)NH—,
—NHC(═O)NH—,
—NH—,
—O—, and
—S—.

42. A pharmaceutical composition comprising the conjugate according to claim 25.

43. The pharmaceutical composition of claim 42 for use as a medicament, for use in the modulation or prophylaxis of the growth of tumour cells, or for use in the treatment of cancer.

44. The pharmaceutical composition for use according to claim 43, wherein the cancer is selected from the group of leukemia, lymphoma, breast cancer, prostate cancer, ovarian cancer, colorectal cancer, gastric cancer, squamous cancer, small-cell lung cancer, head- and-neck cancer, multidrug resistant cancer, glioma, melanoma, and testicular cancer.
